SEQUENCE LISTING (2 sequences)

SEQ ID NO: 1 - DNA, 1745 bp, Homo sapiens
gcgcccctggcagccttcaacgtcggtccccaggcagcatggtgaggtctgctcccggaccctcgccaccatgtacgtgagctacctcctggacaaggacgtgagcatgtaccctagctccgtgcgccactctggcggcctcaacctggcgccgcagaacttcgtcagccccccgcagtacccggactacggcggttaccacgtggcggccgcagctgcagcgcagaacttggacagcgcgcagtccccggggccatcctggccggcagcgtatggcgccccactccgggaggactggaatggctacgcgcccggaggcgcggccgccgccaacgccgtggctcacgcgctcaacggtggctccccggccgcagccatgggctacagcagccccgcagactaccatccgcaccaccacccgcatcaccacccgcaccacccggccgccgcgccttcctgcgcttctgggctgctgcaaacgctcaaccccggccctcctgggcccgccgccaccgctgccgccgagcagctgtctcccggcggccagcggcggaacctgtgcgagtggatgcggaagccggcgcagcagtccctcggcagccaagtgaaaaccaggacgaaagacaaatatcgagtggtgtacacggaccaccagcggctggagctggagaaggagtttcactacagtcgctacatcaccatccggaggaaagccgagctagccgccacgctggggctctctgagaggcaggttaaaatctggtttcagaaccgcagagcaaaggagaggaaaatcaacaagaagaagttgcagcagcaacagcagcagcagccaccacagccgcctccgccgccaccacagcctccccagcctcagccaggtcctctgagaagtgtcccagagcccttgagtccggtgtcttccctgcaagcctcagtgtctggctctgtccctggggttctggggccaactgggggggtgctaaaccccaccgtcacccagtgacccaccggggtctgcagcggcagagcaattccaggctgagccatgaggagcgtggactctgctagactcctcaggagagacccctcccctcccacccacagccatagacctacagacctggctctcagaggaaaaatgggagccaggagtaagacaagtgggatttggggcctcaagaaatatactctcccagatttttactttttccatctggctttttctgccactgaggagacagaaagcctccgctgggcttcattccggactggcagaagcattgcctggactgaccacaccaaccagcttcatctatccgactcttctcttcctagatctgcaggctgcacctctggctagagccgaggggagagagggactcaagggaaaggcaagcttgaggccaagatggctgctgcctgctcatggccctcggaggtccagctgggcctcctgcctccgggcagcaaggtttacactgcggaacgcaaaggcagctaagatagaaagctggactgaccaaagactgcagaacccccaggtggccctgcgtcttttttctcttccctttcccagaccaggaaaggcttggctggtgtatgcacagggtgtggtatgagggggtggttattggactccaggcctgaccagggggcccgaacaggacttgttagagagcctgtcaccagagcttctctgggctgaatgtatgtcagtgctataaatgccagagccaacctgg

SEQ ID NO: 2 - protein, 311 aa, Homo sapiens
MYVSYLLDKDVSMYPSSVRHSGGLNLAPQNFVSPPQYPDYGGYHVAAAAAAAANLDSAQSPGPSWPAAYGAPLREDWNGYAPGGAAAAANAVAHGLNGGSPAAAMGYSSPADYHPHHHPHHHPHHPAAAPSCASGLLQTLNPGPPGPAATAAAEQLSPGGQRRNLCEWMRKPAQQSLGSQVKTRTKDKYRVVYTDHQRLELEKEFHYSRYITIRRKAELAATLGLSERQVKIWFQNRRAKERKINKKKLQQQQQQQPPQPPPPPPQPPQPQPGPLRSVPEPLSPVSSLQASVSGSVPGVLGPTGGVLNPTV

(19) United States
(12) Patent Application Publication
(10) Pub. No.: US 2018/0094322 A1
(43) Pub. Date: Apr. 5, 2018

(54) Biomarker for Predicting Colon Cancer Responsiveness to Anti-Tumor Treatment

(71) Applicant: The Trustees of Columbia University in the City of New York, New York, NY (US)

(72) Inventors: Piero Delfino Dalerba, New York, NY (US); Michael F. Clarke, Menlo Park, CA (US); Debashis Sahoo, San Diego, CA (US); William Joseph Raab, New York, NY (US); Luis Enrique Valencia Salazar, New York, NY (US)

(21) Appl. No.: 15/684,687

(22) Filed: Aug. 23, 2017

Related U.S. Application Data
(60) Provisional application No. 62/378,653, filed on Aug. 23, 2016.
(60) Provisional application No. 62/395,075, filed on Sep. 15, 2016.

(51) Int. Cl. C12Q 1/6886; C12Q 1/6806; C12Q 1/6809; G01N 33/15; C12Q 1/686; A61P 35/00

(52) U.S. Cl. CPC C12Q 1/6886; C12Q 1/6806; C12Q 1/6809; G01N 33/15; C12Q 1/686; C12Q 2600/118; C12Q 2600/16; C12Q 2600/158; G01N 2800/50; G01N 2800/7028; C12Q 2600/106; A61P 35/00

(57) ABSTRACT

The present invention provides a biomarker, namely CDX2, and surrogate CDX2 biomarkers, the expression level of which is useful in predicting response of cancer patients to therapy with an EGFR inhibitor.

### RELATED APPLICATIONS

This application claims the benefit of priority to U.S. Provisional Patent Application No. 62/378,653, filed on Aug. 23, 2016, and U.S. Provisional Patent Application No. 62/395,075, filed on Sep. 15, 2016. The entire contents of these applications are incorporated herein by reference.

### GRANT INFORMATION

This invention was made, in part, with the support of the United States (U.S.) government, under Grants No. K99-CA151673, ROO-CA151673, and TL1-TR001875, awarded by the National Institutes of Health (NIH). The U.S. government has certain rights in the invention.

### SEQUENCE LISTING

The instant application contains a Sequence Listing which has been submitted electronically in ASCII format and is hereby incorporated by reference in its entirety. Said ASCII copy, created on Nov. 20, 2017, is named 123756-00303_SL.txt and is 5618 bytes in size.

### BACKGROUND

Colorectal cancer (CRC) is the third most common form of cancer and the second leading cause of death among cancers worldwide, with approximately 1,000,000 new cases of CRC and 50,000 deaths related to CRC each year (Bandres E, et al., World J Gastroenterol 2007, 13(44):5888-5901; Kim H-J, et al., BMB Rep 2008, 41(10):685-692).

Despite increased availability of novel and effective anti-tumor agents, the design of therapeutic algorithms for optimal treatment of colon cancer patients remains frustrated by the lack of robust predictive biomarkers, which still causes current guidelines to expose many advanced stage (Stage-IV) patients to treatment toxicities and unnecessary costs. For example, therapeutic antibodies targeting the epidermal growth factor receptor (EGFR), for example cetuximab or panitumumab, have been used in clinical practice since 2004, and have proven to be effective therapeutics for the treatment of colorectal cancer. Unfortunately, however, only 8-10% of patients with Stage IV metastatic colorectal cancer respond to such anti-EGFR antibody therapy (Siena, et al. 2009 J Natl Cancer Inst 101(19): 1308-24). It has been demonstrated that certain mutations in the KRAS, NRAS, BRAF and EGFR genes are associated with resistance to treatment with anti-EGFR monoclonal antibodies (Misale et al., Cancer Discovery, 4:1269-1280, 2014; Douillard et al., The New England Journal of Medicine, 369:1023-1034, 2013; Amado et al., Journal of Clinical Oncology, 26:1626-1634, 2008; Karapetis et al., The New England Journal of Medicine, 359:1757-1765, 2008; Di Nicolantonio et al., Journal of Clinical Oncology, 26:5705-5712, 2008; Loupakis et al., British Journal of Cancer, 101:715-721, 2009; Arena et al., Clinical Cancer Research, 21:2157-2166, 2015). While 50% of the CRC population carry mutations in one of these genes, the KRAS mutations are of particular medical significance as they occur in 35%-45% of colorectal cancer patients (Siena, et al. 2009); NRAS and BRAF mutations occur in less than 10% of colorectal cancer patients. Routine testing for the presence of KRAS, NRAS and BRAF mutations is recommended by the National Comprehensive Cancer Network (NCCN) and the American Society of Clinical Oncology (ASCO) for all patients with CRC. Patients with Stage-IV CRCs harboring wild-type (wt) KRAS, NRAS and BRAF genes are considered ideal candidates for anti-EGFR therapy, as they have been shown to benefit from treatment regimens that incorporate cetuximab or panitumumab. However, even among patients with tumors that do not carry KRAS, NRAS or BRAF mutations (i.e., KRAS^{wild-type}, BRAF^{wild-type}) the percentage of those who respond to and/or benefit from treatment with anti-EGFR monoclonal antibodies remains relatively low (15-20%). Moreover, in a recent study (Douillard et al., NEJM, 369:1023-1034, 2013), it was observed that in colon cancer patients with tumors characterized by mutations in either the KRAS or NRAS gene, the addition of anti-EGFR antibody panitumumab to multi-agent chemotherapy (e.g., FOLFOX) is associated with a statistically significant reduction in both progression-free survival (PFS) and overall survival (OS) as compared to treatment with a multi-agent chemotherapy alone. This observation suggests that in patients with tumors that are intrinsically resistant to anti-EGFR monoclonal antibodies, treatment with such antibodies might not only be ineffective (and thus expose patients to unnecessary side-effects and financial costs), but may also cause direct harm in terms of accelerated disease progression and reduced survival (Berlin, NEJM, 369:1059-1060, 2013).

Finally, although BRAF mutations are known to associate with a low probability of CRC response to anti-EGFR antibodies, this association is not absolute. It is known, for example, that certain types of BRAF mutations (e.g., G596R) do not cause, in and of themselves, CRC resistance to anti-EGFR monoclonal antibodies (Yao et al., Nature, 548:234-238, 2017). It also appears that even the most common and most extensively studied type of BRAF mutation (i.e., V600E), though usually associated with reduced survival outcomes and reduced probability of response to anti-EGFR monoclonal antibodies in CRC patients, does not represent, in and of itself, a predictive biomarker of lack of treatment benefit (Rowland et al., British Journal of Cancer, 112:1888-1894, 2015). Even among CRC patients with BRAF V600E mutations, for example, treatment with anti-EGFR monoclonal antibodies often appears to associate with a trend towards improved survival outcomes (Bokemeyer et al., European Journal of Cancer, 48:1466-1475, 2012; Douillard et al., NEJM, 369:1023-1034, 2013). Although the magnitude of such improvements is usually not significant from a statistical point of view, it is also sufficient to be statistically not-inferior than that observed in the case of CRC patients without BRAF V600E mutations (Rowland et al., British Journal of Cancer, 112:1888-1894, 2015). These observations suggest that, even among CRCs with BRAF mutations, a subset may be present that is responsive to anti-EGFR monoclonal antibodies, and could benefit from treatment with such drugs. This concept is also indirectly supported by the observation that CRCs with BRAF V600E mutations represent a biologically heterogeneous family of tumors, which appear to include at least two distinct molecular subtypes, characterized by the differential activation of distinct modules of the EGFR signaling pathway (Barras et al., Clinical Cancer Research, 23:104-115, 2017).

Furthermore, it has been observed that CRC patients with tumors originated from the right side of the colon (e.g., the caecum, ascending colon, transverse colon up to the splenic flexure) have a low probability of benefiting from treatment with anti-EGFR monoclonal antibodies, as compared to CRC patients with tumors originating from the left side of the colon (e.g., the descending colon distal to the spelenic flexure, sigmoid colon, rectum; Brulé et al., European Journal of Cancer, 51:1405-1414, 2015; Boeckx et al., Annals of Oncology, 28:1862-1868, 2017). Based on these observation, the origin of CRCs in the right vs. left side of the colon, a parameter often referred to as “tumor sidedness”, has recently been incorporated in the NCCN clinical guidelines for the first-line treatment of metastatic CRCs, whereby patients with KRAS^{wild-type}, NRAS^{wild-type} forms of the disease are considered eligible for treatment with anti-EGFR monoclonal antibodies only if the tumors are originated in the left side (i.e., between the splenic flexure and the rectum). It is widely recognized, however, that tumor sidedness represents “ . . . , a surrogate for the non-random distribution of molecular subtypes across the colon . . . ” (NCCN Evidence Blocks™, Colon Cancer, v2.2017), and that, in the future, it should be replaced by biomarkers that are more reflective of the mechanistic reasons of the tumors' drug-resistance, and therefore more accurate. Such additional biomarkers are needed to achieve two aims: a) to identify patients with right-sided KRAS^{wild-type}, NRAS^{wild-type} CRCs that are sensitive to anti-EGFR monoclonal antibodies (and therefore should not be excluded from treatment combinations that include such drugs); and b) to identify patients with left-sided KRAS^{wild-type}, NRAS^{wild-type} CRCs that are insensitive to anti-EGFR monoclonal antibodies (and therefore should be excluded from treatment combinations that include such drugs).

Thus, additional predictive biomarkers complementary to KRAS, NRAS, and BRAF are needed in order to optimize the use of anti-EGFR monoclonal antibodies in CRC patients. Additional predictive biomarkers will improve clinical decision-making, enable personalized CRC treatment, further reduce unnecessary toxicity and negative effects on disease progression and survival, and reduce costs associated with treatment of non-responsive patients with anti-EGFR monoclonal antibodies.

### SUMMARY

The present invention is based, at least in part, on the discovery that the biomarker CDX2 (“caudal type homeobox 2”), either alone or in combination with one or more additional biomarkers, is predictive of responsiveness of colorectal cancer (CRC) to treatment with an EGFR inhibitor, e.g., an anti-EGFR antibody. Accordingly, the present invention provides methods for identifying CRC patients who are either responsive or non-responsive (resistant) to treatment with an EGFR inhibitor, e.g., an anti-EGFR antibody such as, for example, cetuximab or panitumumab). The present invention is also based, at least in part, on the identification of biomarkers which have expression patterns which are linearly correlated to CDX2, and, thus, are surrogate biomarkers for CDX2. Therefore, these surrogate biomarkers, which are set forth in Table 1 and Table 2, are also useful (alone or in combination with CDX2) in assessing and predicting responsiveness of cancer, e.g., CRC, to treatment with an EGFR inhibitor, e.g., anti-EGFR monoclonal antibodies cetuximab and panitumumab.

Accordingly, in one aspect, the present invention provides a method of predicting whether a subject diagnosed with colorectal cancer is likely to be responsive or non-responsive to treatment with an EGFR inhibitor, comprising determining the subject's CDX2 expression level, and/or the expression level of one or more surrogate biomarkers set forth in Table 1 or Table 2, in a biological sample obtained from the subject; wherein a CDX2 positive expression level, and/or a positive expression level of one or more surrogate biomarkers set forth in Table 1 or Table 2, indicates that the subject is likely to be responsive to treatment with an EGFR inhibitor and a CDX2 negative expression level, and/or a negative expression level of one or more surrogate biomarkers set forth in Table 1 or Table 2, indicates that the subject is likely to be non-responsive to treatment with an EGFR inhibitor.

In another aspect, the present invention provides a method of assessing the efficacy of an EGFR inhibitor for treating colorectal cancer in a subject prior to administration of the therapeutic agent, comprising determining the subject's CDX2 expression level, and/or the expression level of one or more surrogate biomarkers set forth in Table 1 or Table 2, in a biological sample obtained from the subject, and predicting that the EGFR inhibitor will be efficacious for treating colorectal cancer when the subject's CDX2 expression level, and/or the expression level of one or more surrogate biomarkers set forth in Table 1 or Table 2, is positive and that the EGFR inhibitor will be non-efficacious for treating colorectal cancer when the subject's CDX2 expression level, and/or the expression level of one or more surrogate biomarkers set forth in Table 1 or Table 2, is negative.

In yet another aspect, the present invention provides a method for excluding a subject diagnosed with colorectal cancer from treatment with an EGFR inhibitor, comprising determining the subject's CDX2 expression level, and/or the expression level of one or more surrogate biomarkers set forth in Table 1 or Table 2, in a biological sample obtained from the subject; and excluding a subject from treatment with an EGFR inhibitor if the subject has a CDX2 negative expression level, and/or the expression level of one or more surrogate biomarkers set forth in Table 1 or Table 2 is negative.

In still another aspect, the present invention provides a method of treating colorectal cancer in a subject, comprising determining the subject's CDX2 expression level, and/or the expression level of one or more surrogate biomarkers set forth in Table 1 or Table 2, in a biological sample obtained from the subject, wherein the subject's CDX2 expression level is defined as CDX2 positive or CDX2 negative, and/or the surrogate biomarker expression level is defined as positive or negative, and administering an EGFR inhibitor when the subject's CDX2 expression level is CDX2 positive, and/or the expression level of one or more surrogate biomarkers set forth in Table 1 or Table 2 is positive.

In one embodiment, the method further comprises administration of one or more anti-cancer agents. In another embodiment, the method further comprises administration of chemotherapy or radiation.

In another aspect, the present invention provides a method of determining a clinical course of therapy for treating colorectal cancer in a subject, comprising determining the subject's CDX2 expression level, and/or the expression level of one or more surrogate biomarkers set forth in Table 1 or Table 2, in a biological sample obtained from the subject, and identifying a clinical course of therapy based on the subject's CDX2 expression level, and/or the expression level of one or more surrogate biomarkers set forth in Table 1 or Table 2, wherein therapy with an EGFR inhibitor is selected when the subject's CDX2 expression level, and/or the expression level of one or more surrogate biomarkers set forth in Table 1 or Table 2, is positive, and therapy with an EGFR inhibitor is not selected when the subject's CDX2 expression level, and/or the expression level of one or more surrogate biomarkers set forth in Table 1 or Table 2, is negative.

In still another aspect, the present invention provides a method of determining a clinical course of therapy for treating colorectal cancer in a subject, comprising determining the subject's CDX2 expression level, and/or the expression level of one or more surrogate biomarkers set forth in Table 1 or Table 2, in a biological sample obtained from the subject, and identifying a clinical course of therapy based on the subject's CDX2 expression level, and/or the expression level of one or more surrogate biomarkers set forth in Table 1 or Table 2, wherein subjects with a CDX2 positive expression level, and/or a positive expression level of one or more surrogate biomarkers set forth in Table 1 or Table 2, are treated with an EGFR inhibitor, while subjects with a CDX2 negative expression level, and/or a negative expression level of one or more surrogate biomarkers set forth in Table 1 or Table 2, are treated with a drug combination including an EGFR inhibitor and a drug able to upregulate CDX2 expression in cancer cells.

In some embodiments of the foregoing aspects, the method further comprises analyzing the mutation status of one or more biomarkers selected from the group consisting of KRAS, NRAS, BRAF, EGFR and PIK3CA.

In another aspect, the present invention provides methods of determining whether a patient with or without one or more mutations in the KRAS, NRAS, BRAF or PIK3CA genes would benefit from therapy with an EGFR inhibitor used in combination with one or more molecules that are considered to be surrogates of EGFR inhibitors or synergistic with EGFR inhibitors, wherein the therapy is selected when the subject's CDX2 expression level is CDX2 positive, and/or when the expression level of one or more surrogate biomarkers set forth in Table 1 or Table 2 is positive, and the therapy is not selected when the subject's CDX2 expression level is CDX2 negative, and/or when the expression level of one or more surrogate biomarkers set forth in Table 1 or Table 2 is negative.

In one embodiment, such synergistic or surrogate molecules comprise BRAF inhibitors (e.g. vemurafenib, dabrafenib), MEK inhibitors (e.g. trametinib or selumetinib), and ERK inhibitors (e.g. SCH772984 or VTX11e).

In some embodiments of the foregoing aspects, the method further comprises determining whether a patient with or without one or more mutations in the BRAF gene would benefit from therapy with an EGFR inhibitor alone or in combination with a BRAF inhibitor, wherein the therapy is selected when the subject's CDX2 expression level is CDX2 positive, and/or when the expression level of one or more surrogate biomarkers set forth in Table 1 or Table 2 is positive, and the therapy is not selected when the subject's CDX2 expression level is CDX2 negative, and/or when the expression level of one or more surrogate biomarkers set forth in Table 1 or Table 2 is negative. In one embodiment, the BRAF inhibitor is vemurafenib or dabrafenib.

In some embodiments of the foregoing aspects, the method further comprises determining whether a patient with or without one or more mutations in the KRAS, NRAS, BRAF or PIK3CA genes would benefit from therapy with an EGFR inhibitor in combination with a MEK inhibitor, wherein the therapy is selected when the subject's CDX2 expression level is CDX2 positive, and/or when the expression level of one or more surrogate biomarkers set forth in Table 1 or Table 2 is positive, and the therapy is not selected when the subject's CDX2 expression level is CDX2 negative, and/or when the expression level of one or more surrogate biomarkers set forth in Table 1 or Table 2 is negative. In one embodiment, the MEK inhibitor is trametinib or selumetinib.

In some embodiments of the foregoing aspects, the method further comprises determining whether a patient with or without one or more mutations in the KRAS, NRAS, BRAF or PIK3CA genes would benefit from therapy with an EGFR inhibitor in combination with an ERK inhibitor, wherein the therapy is selected when the subject's CDX2 expression level is CDX2 positive, or when the expression level of one or more surrogate biomarkers set forth in Table 1 or Table 2 is positive, and the therapy is not selected when the subject's CDX2 expression level is CDX2 negative, or when the expression level of one or more surrogate biomarkers set forth in Table 1 or Table 2 is negative. In one embodiment, the ERK inhibitor is SCH772984 or VTX11e.

In some embodiments of the foregoing aspects, the method further comprises determining whether a patient with or without one or more mutations in the KRAS, NRAS, BRAF or PIK3CA genes would benefit from therapy with combinations of multiple synergistic inhibitors of the EGFR signaling pathway and its downstream targets, wherein the therapy is selected when the subject's CDX2 expression level is CDX2 positive, and/or when the expression level of one or more surrogate biomarkers set forth in Table 1 or Table 2 is positive, and the therapy is not selected when the subject's CDX2 expression level is CDX2 negative, and/or when the expression level of one or more surrogate biomarkers set forth in Table 1 or Table 2 is negative.

In one embodiment, synergistic inhibitors of the EGFR signaling pathway and its downstream targets comprise, for example, EGFR inhibitors, BRAF inhibitors (e.g., vemurafenib, dabrafenib), MEK inhibitors (e.g., trametinib or selumetinib) and ERK inhibitors (e.g., SCH772984, VTX11e).

In some embodiments, the methods of the invention further comprise assessing whether the CRC originated on the right side or the left side of the colon. In one embodiment, the right side comprises the caecum, the ascending colon and the transverse colon up to the splenic flexure. In another embodiment, the left side comprises the descending colon distal to the splenic flexure, the sigmoid colon and the rectum. In another embodiment, the CRC originated on the right side. In still another embodiment, the CRC originated on the left side.

In some embodiments, the methods of the invention further comprise obtaining a biological sample from the subject. In one embodiment, the biological sample is a colorectal tumor sample, e.g., obtained from a tissue biopsy. In some embodiments, the tumor sample is a fixed, paraffin-embedded tissue sample. In another embodiment, the sample is a blood sample, e.g., a serum sample.

In some embodiments of the foregoing aspects, a positive CDX2 expression level, or surrogate biomarker expression level, is indicated by a measureable level of CDX2 expression, or surrogate biomarker expression, in the biological sample.

In other embodiments of the foregoing aspects, a positive CDX2 expression level, or surrogate biomarker expression level, is indicated by a level of CDX2 expression, or surrogate biomarker expression, in the biological sample that is greater than or equal to a threshold level chosen to separate samples with low levels of CDX2 expression (CDX2^{low}) from samples with high levels of CDX2 expression (CDX2^{high}) or to separate samples with low levels of surrogate biomarker expression from samples with high levels of surrogate biomarker expression.

In still other embodiments of the foregoing aspects, a positive CDX2 expression level, or surrogate biomarker expression level, is indicated by a level of CDX2 expression, or surrogate biomarker expression, in the biological sample that is greater than a threshold level chosen to separate samples with low levels of CDX2 expression (CDX2^{low}) from samples with high levels of CDX2 expression (CDX2^{high}) or to separate low levels of surrogate biomarker expression from samples with high levels of surrogate biomarker expression.

In some embodiments, the threshold level used to separate samples with low levels of CDX2 expression (CDX2^{low}), or low surrogate biomarker expression from samples with high levels of CDX2 expression (CDX2^{high}) or high surrogate biomarker expression, is chosen based on a mathematical approach that assumes a bimodal distribution of the CDX2 expression values, or surrogate biomarker expression values. In one embodiment, the mathematical approach used to separate CDX2^{neg/low} from CDX2^{high} is the StepMiner algorithm.

In some embodiments, the threshold level used to separate samples with low levels of CDX2 expression (CDX2^{low}), or low levels of surrogate biomarker expression, from samples with high levels of CDX2 expression (CDX2^{high}) or high levels of surrogate biomarker expression, is chosen using an empirical approach designed to identify the threshold level below which a treatment with EGFR inhibitors is not associated with an improvement in clinical outcome. In some embodiments of the foregoing aspects, an improvement in clinical outcome is defined as an increase in objective clinical responses (OCR) or overall response rates (ORR), an increase in progression free survival (PFS), an increase in time-to-recurrence (TTR), an increase in time-to-treatment failure (TTF), an increase in disease-free survival (DFS), an increase in relapse-free survival (RFS), an increase in overall survival (OS), an increase in disease-specific survival (DSS) or cancer-specific survival (CSS), and/or an increase in quality adjusted life years (QALY).

In some embodiments of the foregoing aspects, a negative CDX2 expression, or negative surrogate biomarker expression is indicated by a lack of CDX2 expression, or lack of surrogate biomarker expression, in the biological sample.

In some embodiments, a negative CDX2 expression level, or negative surrogate biomarker expression, is indicated by a level of CDX2 expression, or surrogate biomarker expression, in the biological sample, that is less than or equal to a threshold level chosen to separate samples with low levels of CDX2 expression (CDX2^{low}) from samples with high levels of CDX2 expression (CDX2^{high}) or to separate low levels of surrogate biomarker expression from samples with high levels of surrogate biomarker expression.

In other embodiments, a negative CDX2 expression level, or surrogate biomarker expression level, is indicated by a level of CDX2 expression, or surrogate biomarker expression, in the biological sample that is less than a threshold level chosen to separate samples with low levels of CDX2 expression (CDX2^{low}) from samples with high levels of CDX2 expression (CDX2^{high}) or to separate samples with low levels of surrogate biomarker expression from samples with high levels of surrogate biomarker expression.

In other embodiments of the foregoing aspects, the subject's CDX2 expression level, or surrogate biomarker expression level, is determined by measuring the level of CDX2 protein expression, or surrogate biomarker protein expression, in the biological sample. In one embodiment, the level of CDX2 expression is measured by contacting the sample with a reagent that specifically binds with the protein. In some embodiments, the reagent is an antibody or antigen-binding fragment thereof, e.g., a monoclonal antibody. In some embodiments, the level of CDX2 protein expression, or surrogate biomarker protein expression, is determined by immunohistochemistry or ELISA. In other embodiments, the level of CDX2 protein expression, or surrogate biomarker protein expression, is determined by HPLC/UV-Vis spectroscopy, mass spectrometry, mass cytometry, NMR, or any combination thereof. In some embodiments, the level of CDX2 protein expression, or surrogate biomarker protein expression, in cancer cells is determined by mass cytometry, either alone or in combination with one or more additional protein markers. In other embodiments, the one or more additional protein markers comprise EPCAM or desmoplakyn (DSP).

In other embodiments of the foregoing aspects, the subject's CDX2 expression level, or surrogate biomarker expression level, is determined by determining the level of its corresponding mRNA in the biological sample. In one embodiment, an amplification reaction is used to determine the level of the mRNA. In some embodiments, a hybridization assay is used to determine the level of the mRNA. In one embodiment, an oligonucleotide complementary to a portion of the mRNA is used in the hybridization assay.

In other embodiments of the foregoing aspects, the EGFR inhibitor is an anti-EGFR antibody, e.g., cetuximab or panitumumab. In other embodiments of the foregoing aspects, the EGFR inhibitor is a small molecule.

In other embodiments of the foregoing aspects, the methods further comprise treating the subject with an anti-EGFR inhibitor when the subject's CDX2 expression level is CDX2 positive, and/or when the expression level of one or more surrogate biomarkers set forth in Table 1 or Table 2 is positive.

In other embodiments of the foregoing aspects, a computer-implemented program is used to compare the subject's CDX2 expression state (and/or the expression state of a surrogate biomarker) to a statistical model-predicted relationship between CDX2 expression level (and/or surrogate biomarker expression level) and the likelihood of a refractory response to treatment with an EGFR inhibitor determined from a population of patients with colorectal cancer treated with an EGFR inhibitor; and generating a report comprising a prediction as to whether the colon cancer is likely to respond to treatment with an EGFR inhibitor.

In other embodiments of the foregoing aspects, the method used to determine the subject's CDX2 expression level includes the determination of CDX2 expression levels in individual cancer cells within the subject's tumor, or a sample thereof, and the calculation of the percentage of CDX2 positive and CDX2 negative cancer cells in the tumor or in the tumor sample.

In other embodiments of the foregoing aspects, the determination of CDX2 expression levels, or surrogate biomarker expression levels, in individual cancer cells within the subject's tumor, or a sample thereof, is performed in a manner that defines cancer cells as CDX2 negative or CDX2 positive based on their individual CDX2 expression levels, or positive or negative for a surrogate biomarker based on the surrogate biomarker(s) expression levels.

In some embodiments, an individual cancer cell is defined as CDX2 positive if the individual CDX2 expression level is greater than a threshold level chosen to separate cells with low levels of CDX2 expression (CDX2^{low}) from cells with high levels of CDX2 expression (CDX2^{high}). In other embodiments, wherein an individual cancer cell is defined as CDX2 positive if its individual CDX2 expression level is greater than or equal to a threshold level chosen to separate cells with low levels of CDX2 expression (CDX2^{low}) from cells with high levels of CDX2 expression (CDX2^{high}). In some embodiments, the threshold level used to separate cells with low levels of CDX2 expression (CDX2^{low}) from cells with high levels of CDX2 expression (CDX2^{high}) is chosen based on a mathematical approach that assumes a bimodal distribution of the CDX2 expression values in individual cells. In one embodiment, the mathematical approach used to separate CDX2^{neg/low} from CDX2^{high} individual cancer cells is the StepMiner algorithm.

In other embodiments of the foregoing aspects, the colorectal cancer is colon cancer, e.g., stage I, stage II, stage III, or stage IV colon cancer. In still other embodiments of the foregoing aspects, the colorectal cancer is rectal cancer, e.g., stage I, stage II, stage III, or stage IV rectal cancer.

In one aspect, the invention provides a kit for predicting whether a subject diagnosed with colorectal cancer is likely to be responsive or non-responsive to treatment with an EGFR inhibitor or assessing the efficacy of a therapeutic agent for treating colorectal cancer, comprising reagents useful for determining the subject's CDX2 expression level (and/or surrogate biomarker expression level) in a biological sample from the subject. In one embodiment, the biological sample is a colorectal tumor sample, e.g., obtained from a tissue biopsy. In another embodiment, the sample is a blood sample, e.g., a serum sample.

In one embodiment, the kit comprises one or more of packaged arrays/microarrays, biomarker-specific antibodies, or beads. In one embodiment, the kit comprises at least one monoclonal antibody or antigen-binding fragment thereof, that specifically binds with CDX2, or a surrogate biomarker, for determining the subject's CDX2 expression level, or surrogate biomarker expression level. In one embodiment, the kit comprises two or more antibodies or antigen-binding fragments thereof, that each specifically bind with CDX2 and/or one or more surrogate biomarker. In other embodiments, the kit comprises further comprising reagents useful for detecting one or more biomarkers selected from the group consisting of KRAS, NRAS, BRAF, EGFR and PIK3CA.

### BRIEF DESCRIPTION OF THE DRAWINGS

FIGS. 1A-F show the relationship between CDX2 mRNA expression and response to cetuximab. The distribution of CDX2 expression levels are shown with regard to the enterocyte differentiation marker SLC26A3 (“Solute Carrier Family 26 Member 3”). FIGS. 1A and 1B represent all patients. FIGS. 1C and 1D represent all patients (stratified for KRAS status). FIGS. 1E and 1F represent KRAS wild-type. CDX2 and SLC26A3 mRNA expression levels are linked by a Boolean relationship: “CDX2-low” implies “SLC26A3-low” (i.e., when CDX2 expression is low, SLC26A3 expression is low), which is mathematically equivalent to “SLC26A3-high” implies “CDX2-high” (i.e., when SLC26A3 expression is high, CDX2 expression is high).

FIGS. 2A-D show an exemplary scoring system for the evaluation of CDX2 protein expression by immunohistochemistry. CDX2 protein expression can be evaluated by immunohistochemistry, using several assays that have been validated for diagnostic applications in clinical laboratories (Borrisholt et al., Appl. Immunohistochem. Mol. Morphol., 21:64-72, 2013). A scoring system that can be used to stratify human colon carcinomas into two distinct subgroups (CDX2^{neg} vs. CDX2^{pos}) based on their nuclear CDX2 protein expression level is described herein and in Dalerba, et al. NEJM, 374:211-222, 2016. According to this system, all tumors whose malignant epithelial component either completely lacks CDX2 expression or shows faint nuclear expression in a minority of malignant epithelial cells were scored as CDX2^{neg}. Tumors scored as CDX2^{neg} fall into two staining patterns: a) complete lack of CDX2 expression (Score 0; FIG. 2A); and b) scattered and faint nuclear expression in a minority fraction of cancer cells (Score 0.5; FIG. 2B). Conversely, all tumors whose malignant epithelial component displays widespread nuclear expression of CDX2 were scored as CDX2^{pos}. Tumors scored as CDX2^{pos} also fall into two staining patterns: a) strong staining in a majority fraction of cancer cells (Score 2; FIG. 2C); and b) strong staining in all cancer cells (Score 3; FIG. 2D). The relative frequency of the various staining patterns was evaluated using a colon cancer tissue-microarray (TMA) from the National Cancer Institute's Cancer Diagnosis Program (NCI-CDP).

FIGS. 3A-B show an exemplary human mRNA sequence (Genbank: U51096; SEQ ID NO:1) (FIG. 3A) and the human peptide (NCBI Reference Sequence: NP_001256.3; SEQ ID NO:2) (FIG. 3B) for caudal-type homeobox gene 2 (CDX2).

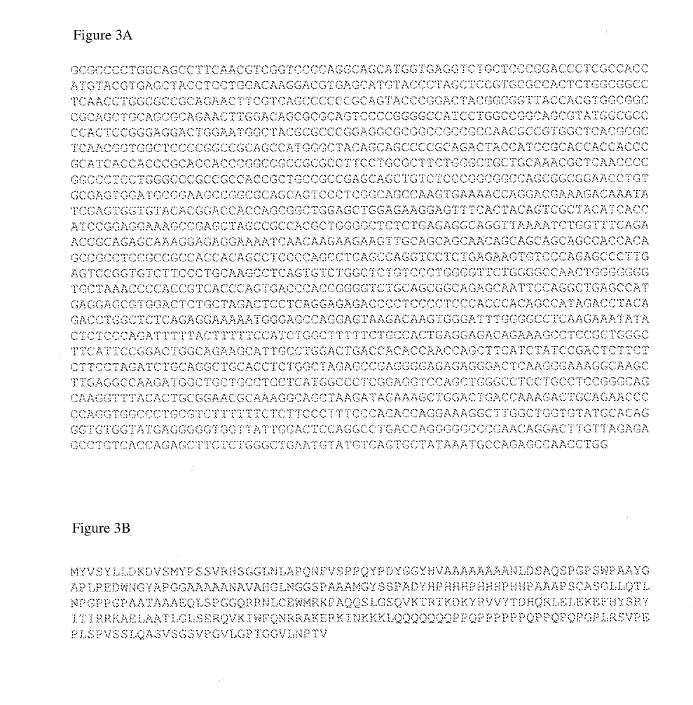

FIGS. 4A-D show high-throughput mining of gene-expression databases using Boolean logic. To identify pairs of genes whose expression is regulated by Boolean implications, the BooleanNet software algorithm (Sahoo et al., Genome Biology, 9:R157, 2008), described herein, was exploited. In this study, a search based on a Boolean implication of the “Xneg implies Ypos” type (FIG. 4A) was performed. Gene-expression patterns were considered to fulfill this type of implication when the false-discovery rate (FDR) of a sparsity test in the lower left quadrant was <0.0001 (10⁻⁴). Threshold gene expression levels were calculated using the StepMiner algorithm, based on the expression distribution of the 47,240 gene-expression arrays contained within the “Human NCBI-GEO Global Database” (FIG. 4B), and an intermediate region (“noise zone”) was defined around each threshold with a width of 1 (i.e. threshold +/−0.5), corresponding to a 2-fold change in expression, which is the minimum noise level in these types of datasets. The fulfillment of the “Xneg implies ALCAMpos” was tested on the “Human Colon Global Database” (n=2.329 samples after “purging” based on the fulfillment of the EpCAMpos/ALBneg condition). Among the genes that fulfilled the “Xneg implies ALCAMpos” relationship was the gene encoding for the homeobox transcription factor CDX2 (FIG. 4C). The threshold gene-expression levels for the lower left quadrant were: 6.67 (i.e. 7.17-0.5) for ALCAM (Affymetrix probe 201951_at) and 6.46 (i.e. 6.96-0.5) for CDX2 (Affymetrix probe 206387_at; FIG. 4D). Gene-expression levels were assigned for each gene in each array, using the log 2 of the expression values.

FIGS. 5A-D show identification of CDX2. A database containing 2,329 human gene expression arrays from both normal colon (n=214), and colorectal cancer tissue samples (n=2115), was mined to identify genes that fulfilled the “Xneg implies ALCAMpos” Boolean implication. A sparsity test for the lower left quadrant was performed, after threshold definition using the StepMiner algorithm and using a false-discovery rate (FDR)<0.0001 (10⁻⁴). This screening yielded 16 candidate genes, that were ranked based on the dynamic range of their gene-expression values (FIG. 5A). Among genes ranking at the top was the homeobox gene CDX2. A visual analysis of CDX2 and ALCAM gene-expression relationships using two-axis scatter plots confirmed the “CDX2neg implies ALCAMpos” Boolean relationship (FIG. 5B). A box-plot analysis (FIG. 5C) indicated that mean ALCAM gene-expression levels were higher in CDX2neg colorectal carcinomas (n=87) as compared to CDX2pos ones (n=2028) and to normal colorectal epithelium (n=214). A 2-sample t-test to compare mean ALCAM gene-expression levels in the three populations indicated that these differences were statistically significant (FIG. 5D).

FIG. 6 illustrates bimodal distribution of CDX2 protein expression in the NCI-CDP tissue micro-array (TMA) dataset of human primary colon carcinomas (n=366). This methodology represents a semi-quantitative assessment of nuclear CDX2 expression in a cancer cell population.

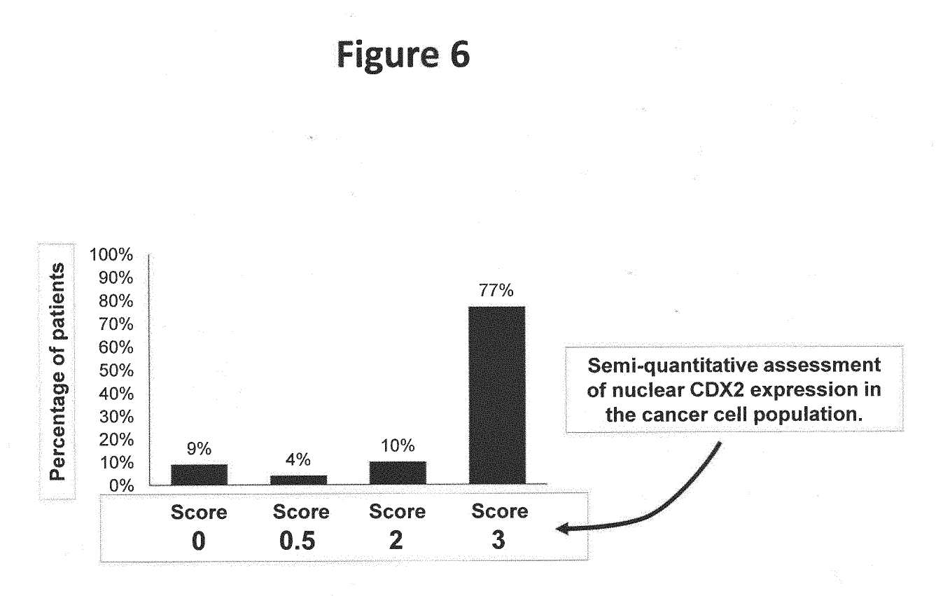

FIGS. 7A-D illustrate the relationship between CDX2 mRNA expression, KRAS mutation status and objective tumor response (OTR) (i.e., objective tumor shrinkage) following treatment with the anti-EGFR monoclonal antibody cetuximab across two independent colon cancer gene-expression datasets (GSE5851, E-MTAB-991). The relationship between CDX2 mRNA expression and objective tumor regression (OTR) following treatment with anti-EGFR monoclonal antibodies was studied in a database of 111 independent colon carcinomas treated with cetuximab monotherapy. The database was obtained by pooling two independent gene expression array datasets: 1) GSE5851, downloaded from the NCBI-GEO public repository, and annotated with OTR information related to 68 primary tissue specimens from Stage-IV metastatic colon carcinomas (Khambata-Ford et al., J. Clin. Oncol., 25:3230-3237, 2007); 2) E-MTAB-991, downloaded from the EMBL-ArrayExpress public repository, and annotated with OTR information related to 43 patient-derived xenograft (PDX) lines (Julien et al., Clin. Cancer Res., 18:5314-5328, 2012). A visual exploration of the distribution of CDX2 and SLC26A3 mRNA expression levels across the two datasets, based on scatter-plots, revealed that tumors undergoing tumor regression were restricted to the CDX2^{pos} subgroup (FIG. 7A, all evaluable tumors; FIG. 7B, KRAS^{wt} evaluable tumors). The association between CDX2 mRNA expression and OTR was tested for statistical significance using 2×2 contingency tables and Fisher's exact probability test, after stratification of tumors in CDX2^{neg} and CDX2^{pos} subgroups using the StepMiner algorithm (Dalerba et al., N. Engl. J. Med., 374:211-222, 2016). Lack of CDX2 mRNA expression was associated with reduced OTR frequency, both across the whole database (FIG. 7C; p<0.01) and within the KRASwt subgroup (FIG. 7D; p=0.02).

FIGS. 8A-D illustrate the relationship between CDX2 mRNA expression, KRAS mutation status and disease control (DC) (i.e. lack of increase in tumor size) following treatment with the anti-EGFR monoclonal antibody cetuximab across two independent colon cancer gene-expression datasets (GSE5851, E-MTAB-991). The relationship between CDX2 mRNA expression and disease control (DC) following treatment with anti-EGFR monoclonal antibodies was studied in a database of 111 independent colon carcinomas treated with cetuximab monotherapy. The database was obtained by pooling two independent gene-expression array datasets: 1) GSE5851, downloaded from the NCBI-GEO public repository, and annotated with DC information related to 68 primary tissue specimens from Stage-IV metastatic colon carcinomas (Khambata-Ford et al., J. Clin. Oncol., 25:3230-3237, 2007); 2) E-MTAB-991, downloaded from the EMBL-Array-Express public repository, and annotated with DC information related to 43 patient-derived xenograft (PDX) lines (Julien et al., Clin. Cancer Res., 18:5314-5328, 2012). A visual exploration of the distribution of CDX2 and SLC26A3 mRNA expression levels across the two datasets, based on scatter-plots, revealed that tumors undergoing DC were mostly found in the CDX2^{pos} subgroup (FIG. 8A, all evaluable tumors; FIG. 8B, KRAS^{wt} evaluable tumors). The association between CDX2 mRNA expression and DC was tested for statistical significance using 2×2 contingency tables and the χ2 test, after stratification of tumors in CDX2^{neg} and CDX2^{pos} subgroups using the StepMiner algorithm (Dalerba et al., N. Engl. J. Med., 374:211-222, 2016). Lack of CDX2 mRNA expression was associated with a reduced frequency of DC, both across the whole database (FIG. 8C; p<0.01) and within the KRAS^{wt} tumor subgroup (FIG. 8D; p=0.03).

### DETAILED DESCRIPTION

EGFR inhibitors such as the anti-EGFR monoclonal antibodies cetuximab and panitumumab have proven to be effective therapies for certain colorectal cancers (CRC). However, many tumors do not respond to treatment with these EGFR inhibitors. The presence of mutations in the KRAS, NRAS, BRAF, EGFR and PIK3CA genes are known to be related to colon cancer resistance to treatment with EGFR inhibitors (i.e., carcinomas that express mutations in one or more of the KRAS, NRAS, BRAF, EGFR or PIK3CA genes are associated with resistance to treatment with EGFR inhibitors). However, even in those tumors that do not express mutations in KRAS, NRAS, BRAF, EGFR or PIK3CA (i.e., tumors that are wild-type for these genes), patient response rates to treatment with anti-EGFR antibodies are low, i.e., only approximately 15%-20%. With respect to BRAF, while mutations in this gene are known to associate with a low probability of CRC response to anti-EGFR antibodies, this association is not absolute (Yao et al., Nature, 548:234-238, 2017; Rowland et al., British Journal of Cancer, 112:1888-1894, 2015; Bokemeyer et al., European Journal of Cancer, 48:1466-1475, 2012; Douillard et al., NEJM, 369:1023-1034, 2013). Thus, even among CRCs with BRAF mutations, a subset of patients may be responsive to anti-EGFR monoclonal antibodies, and could benefit from treatment with such drugs.

Moreover, a parameter referred to as “tumor sidedness” (i.e., whether a colon tumor originated on the left side or the right side) has been determined to be related to drug responsiveness, wherein CRC patients with tumors originating from the right side of the colon have a low probability of benefiting from treatment with anti-EGFR monoclonal antibodies, as compared to CRC patients with tumors originating from the left side of the colon (Brulé et al., European Journal of Cancer, 51:1405-1414, 2015; Boeckx et al., Annals of Oncology, 28:1862-1868, 2017). Based on these observations, the origin of CRCs in the right vs. left side of the colon has recently been incorporated in the National Comprehensive Cancer Network (NCCN) clinical guidelines for the first-line treatment of metastatic CRCs, whereby patients with KRAS^{wild-type} NRAS^{wild-type} forms of the disease are considered eligible for treatment with anti-EGFR monoclonal antibodies only if the tumors are originated in the left side (i.e., between the splenic flexure and the rectum). However, tumor sidedness in combination with KRAS and NRAS status is not a perfect predictor of drug resistance, e.g., resistance to treatment with EGFR inhibitors (NCCN Evidence Blocks™, Colon Cancer, v2.2017). Thus, additional markers are needed to stratify these populations (CRCs having wild-type or mutated KRAS, NRAS, BRAF, EGFR or PIK3CA, and left- and right-sided originating tumors) for responsiveness and non-responsiveness.

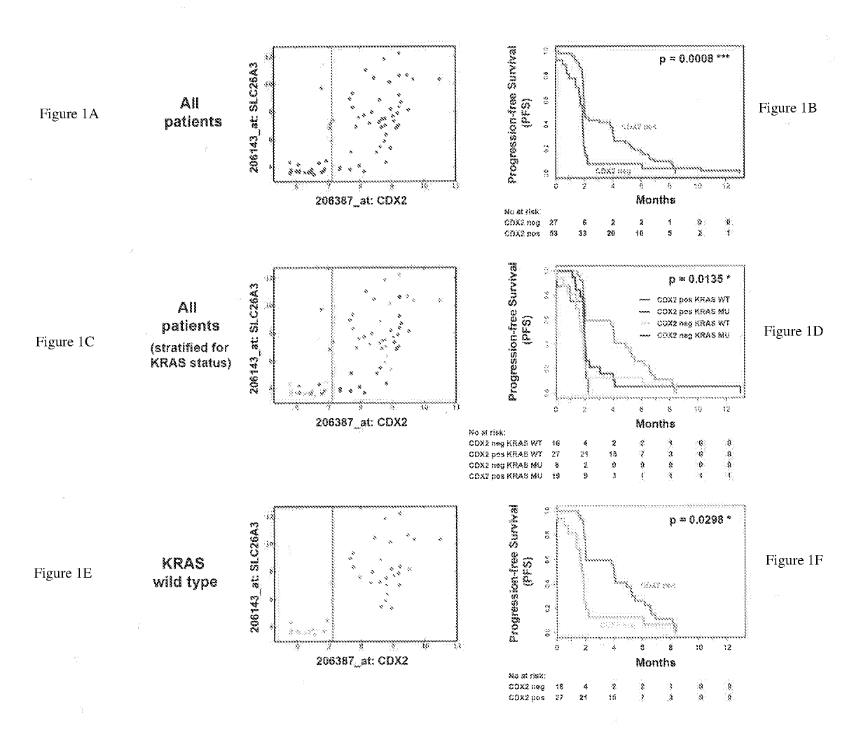

Accordingly, the present invention is based, at least in part, on the identification of a biomarker useful in assessing and predicting responsiveness of cancer, e.g., CRC, to treatment with an EGFR inhibitor, e.g., anti-EGFR monoclonal antibodies cetuximab and panitumumab. In particular, the present invention relates to the identification of the transcription factor “caudal type homeobox 2” (CDX2) as a biomarker for the effectiveness of treatment with an EGFR inhibitor, wherein either lack of CDX2 expression or low CDX2 expression levels are correlated with non-responsiveness to therapy with an EGFR inhibitor, e.g., cetuximab or panitumumab. As described in Example 1, below, the present inventors have determined that human colon carcinomas either lacking expression or having low levels of expression (i.e., protein expression or mRNA expression) of CDX2, are intrinsically resistant to the anti-tumor activity of the anti-EGFR monoclonal antibody cetuximab, irrespective of their KRAS mutation status (see FIGS. 1A-F).

The present invention is also based, at least in part, on the identification of biomarkers which have expression patterns which are linearly correlated to CDX2, and, thus, are surrogate biomarkers for CDX2 (see Example 2). Therefore, these surrogate biomarkers, which are set forth in Table 1 and Table 2, are also useful in assessing and predicting responsiveness of cancer, e.g., CRC, to treatment with an EGFR inhibitor, e.g., anti-EGFR monoclonal antibodies cetuximab and panitumumab. Table 1, set forth below, includes a list of surrogate biomarkers whose mRNA expression levels were identified as positively correlated to those of CDX2. Table 2, also set forth below, includes a list of surrogate biomarkers from Table 1 whose “high” expression levels associate with a statistically significant benefit from cetuximab monotherapy in KRAS^{wt} colon cancer patients. The biomarkers set forth in Table 1 and Table 2 are referred to herein as “surrogate biomarkers” or “surrogate CDX2 biomarkers.”

One or more of the surrogate biomarkers described in Table 1 and Table 2 can be used alone or in combination with CDX2 to assess and predict responsiveness of colorectal cancer to treatment with an EGFR inhibitor, e.g., cetuximab and panitumumab.

Some of the genes included in Table 1 and Table 2 encode for proteins that can be “shed” by tumor cells in the bloodstream, and therefore can become measurable in the circulation, thus serving as serum biomarkers. A representative example of a biomarker that is detectable in serum is CEACAM5 (also known as CEA), set forth in Table 1, which is detectable in the circulation of patients with metastatic colon cancer.

Thus, in certain non-limiting aspects, the present invention provides methods for predicting whether a subject diagnosed with colorectal cancer is likely to be non-responsive (i.e., refractory or resistant) to treatment with an EGFR inhibitor, comprising determining the subject's CDX2 expression level, and/or the expression level of one or more surrogate biomarkers set forth in Table 1 or Table 2, in a biological sample obtained from the subject, wherein a CDX2 negative expression level, and/or a negative expression level of a surrogate biomarker set forth in Table 1 or Table 2, indicates that the subject is likely to be non-responsive to treatment with an EGFR inhibitor. In one embodiment, the patient is then excluded from treatment with an EGFR inhibitor.

In other non-limiting aspects, the present invention provides a method for predicting whether a subject diagnosed with colorectal cancer is likely to be responsive to treatment with an EGFR inhibitor, comprising determining the subject's CDX2 expression level, and/or the expression level of one or more surrogate biomarkers set forth in Table 1 or Table 2, in a biological sample obtained from the subject, wherein a CDX2 positive expression level, and/or a positive expression level of a surrogate biomarker set forth in Table 1 or Table 2, indicates that the subject is likely to be responsive to treatment with an EGFR inhibitor. In one embodiment, the patient is identified as a candidate for treatment with an EGFR inhibitor. In another embodiment, a therapeutically effective amount of an EGFR inhibitor, alone or in combination with one or more additional anti-cancer therapeutic agents, is administered to the patient to treat the colorectal cancer.

In another non-limiting aspect, the present invention provides methods of treating colorectal cancer in a subject, comprising determining the subject's CDX2 expression level, and/or the expression level of one or more surrogate biomarkers set forth in Table 1 or Table 2, in a biological sample obtained from the subject and administering a therapeutically effective amount of an EGFR inhibitor when the subject's CDX2 expression level is positive, and/or wherein the expression level of one or more surrogate biomarkers set forth in Table 1 or Table 2 is positive.

In one embodiment, of one or more additional anti-cancer therapeutic agents can be administered to the patient (either sequentially or concurrently), including, but not limited, to chemotherapy or radiation. Exemplary additional anti-cancer agents include, but are not limited to: 1) alkylating agents, including, but not limited to: a] nitrogen mustards, including, but not limited to mechlorethamine, cyclophosphamide, ifosfamide, chlorambucil, melphalan, busulfan, alone or in combination with sodium 2-sulfanylethanesulfonate (Mesna); b] nitrosoureas, including, but not limited to N-Nitroso-N-methylurea (MNU), carmustine (BCNU), lomustine (CCNU) and semustine (MeCCNU), fotemustine and streptozotocin; c] tetrazines, including, but not limited to dacarbazine, mitozolomide, and temozolomide; d] aziridines, including, but not limited to thiotepa, mytomycin and diaziquone (AZQ); e] platinum coordination compounds, including, but not limited to cisplatin, carboplatin, oxaliplatin; f] other alkylating agents, including, but not limited to procarbazine, and hexamethylmelamine; 2) anti-metabolites, including, but not limited to: a] inhibitors of dyhydrofolate reductase (DHFR) as well as inhibitors of other enzymes involved in folate metabolism, including, but not limited to methotrexate, pemetrexed and raltritrexed; b] fluoropyrimidines as well as other inhibitors of thymidylate synthase (TS), including, but not limited to 5-fluorouracil alone or in combination with leucovorin, capecitabine, floxuridine, tegafur (UFT, UFUR) and trifluridine alone or in combination with inhibitors of thymidine phosphorylase, such as tipiracil; c] deoxynucleoside analogues including, but not limited to cytarabine, gemcitabine, decitabine, fludarabine, nelarabine, cladribine, clofarabine and pentostatin; d] ribonucleoside analogues including, but not limited to azacitidine, e] thiopurines including, but not limited to thioguanine and mercaptopurine; 3) inhibitors of the microtubule function, including, but not limited to: a] vinca alkaloids, including, but not limited to vincristine, vinblastine, vindesine and vinorelbine; b] taxanes including, but not limited to paclitaxel, docetaxel and cabazitaxel; c] analogs of epothilone B, including, but not limited to ixabepilone; 4) inhibitors of topoisomerase, including, but not limited to: a] inhibitors of topoisomerase I, including, but not limited to irinotecan and topotecan; b] inhibitors of topoisomerase II, including, but not limited to etoposide, teniposide, doxorubicin, novobiocin, bleomycin, merbarone and mitoxantrone; and 5) cytotoxic antibiotics, including, but not limited to: a] anthracyclins, including, but not limited to doxorubicin (adriamycin), daunorubicin, idarubicin, epirubicin, pirarubicin, iododoxorubicin, nemorubicin, and aclarubicin, either alone or in liposomal formulations; b] other cytotoxic antibiotics, including, but not limited to bleomycin, mitomycin C, mitoxantrone, actinomycin; 6) other miscellaneous cytotoxic compounds, including, but not limited to trabectedin (ecteinascidin 743, ET-743); 7) inhibitors of angiogenesis, including, but not limited to: a] anti-VEGF monoclonal antibodies, including, but not limited to bevacizumab; b] anti-VEGFR monoclonal antibodies, including, but not limited to ramucirumab; c] recombinant, chimeric, soluble and/or re-engineered versions of VEGFR, including, but not limited to aflibercept; d] inhibitors of VEGFR tyrosine kinase activity, including, but not limited to regorafenib, sorafenib, pazopanib, and sunitinib; 8) immune check-point inhibitors, including, but not limited to: a] anti-CTLA4 monoclonal antibodies, including, but not limited to ipilimumab and tremelimumab; b] anti-PD1 monoclonal antibodies, including, but not limited to nivolumab and pembrolizumab; c] anti-PDL1 monoclonal antibodies, including, but not limited to atezolizumab; 9) inhibitors of HER2, including, but not limited to; a] anti-HER2 monoclonal antibodies, including, but not limited to trastuzumab and pertuzumab, either alone or in combination (e.g. trastuzumab+pertuzumab) or conjugated to cytotoxins or radionucliudes; b] inhibitors of HER2 tyrosine kinase activity, including, but not limited to lapatinib; 11) anti-RANKL monoclonal antibodies, including, but not limited to denosumab; 12) inhibitors of BRAF tyrosine kinase activity, including, but not limited to vemurafenib, and dabrafenib; 13) inhibitors of MEK tyrosine kinase activity, including, but not limited to trametinib and selumetinib; 14) inhibitors of ALK tyrosine kinase activity, including, but not limited to crizotinib; 15) inhibitors of MET tyrosine kinase activity, including, but not limited to cabozantinib; 16) inhibitors of KIT tyrosine kinase activity, including, but not limited to imatinib and dasatinib; 17) inhibitors of ABL tyrosine kinase activity, including, but not limited to imatinib, dasatinib, nilotinib and ponatinib; 18) inhibitors of CDK tyrosine kinase activity, including, but not limited to palbociclib and amenaciclib; 19) inhibitors of COX1 enzymes, including, but not limited to acetyl-salicylic acid, naproxene, ibuprofen, indomethacyn, and diclofenac; 20) inhibitors of COX2 enzymes, including, but not limited to celecoxib and rofecoxib; 21) inhibitors of PARP enzymes, including, but not limited to olaparib, niraparib and veliparib; and 22) others.

In another embodiment, where the subject diagnosed with colorectal cancer is CDX2 positive or CDX2 negative, an EGFR inhibitor is administered with at least one additional therapeutic agent (either sequentially or concurrently), wherein the additional therapeutic agent is capable of disabling the tumor resistance mechanisms, thus restoring the tumor's sensitivity to the EGFR inhibitor.

In another embodiment, where the subject diagnosed with colorectal cancer has a positive or negative expression level of one or more surrogate biomarkers set forth in Table 1 or Table 2, an EGFR inhibitor is administered with at least one additional therapeutic agent (either sequentially or concurrently), wherein the additional therapeutic agent is capable of disabling the tumor resistance mechanisms, thus restoring the tumor's sensitivity to the EGFR inhibitor. In one embodiment, the one or more surrogate biomarkers are used alone or in combination with CDX2.

Moreover, the present inventors have also determined that CDX2 negative colon carcinomas are refractory to treatment with an EGFR inhibitor irrespective of the presence of a mutation in KRAS (i.e., resistance of CDX2 negative colon carcinomas to treatment with an EGFR inhibitor is observed even in carcinomas that are express wild-type KRAS). Thus, the use of CDX2 (and/or a CDX2 surrogate biomarker set forth in Table 1 or Table 2) as a biomarker for resistance to treatment with EGFR inhibitors is non-redundant with the information provided by other biomarkers such as KRAS, NRAS, BRAF, EGFR or PIK3CA. Therefore, in another aspect, the CDX2 biomarker of the present invention, and/or one or more surrogate biomarker of CDX2, can be used alone or in combination with the mutation status of one or more of KRAS, NRAS, BRAF, EGFR or PIK3CA (or other biomarkers used to predict the responsiveness of cancer to a therapeutic agent) to identify CRC patients which are responsive to EGFR inhibitor treatment and could benefit from treatment with an EGFR inhibitor, and/or which CRC patients are resistant to treatment with an EGFR inhibitor and should be excluded from treatment with an EGFR inhibitor.

For example, in one embodiment, a colorectal carcinoma that is negative for CDX2 expression and expresses one or more of wild-type KRAS, NRAS, BRAF, EGFR or PIK3CA, is predicted to be resistant to EGFR inhibitor treatment. In another embodiment, a colon carcinoma that is positive for CDX2 expression and expresses one or more of mutant KRAS, NRAS, BRAF, EGFR or PIK3CA, is predicted to be responsive to treatment with an EGFR inhibitor.

In another embodiment, CDX2, alone or in combination with one or more surrogate biomarkers set forth in Table 1 or Table 2, can be used to identify patients with right-sided KRAS^{wild-type}, NRAS^{wild-type} CRCs that are responsive to anti-EGFR monoclonal antibodies (and therefore should not be excluded from treatment combinations that include such drugs). For example, the present invention provides a method for predicting whether a subject diagnosed with colorectal cancer who is KRAS^{wild-type}, NRAS^{wild-type} and has a right-side originating colon tumor, is likely to be responsive to treatment with an EGFR inhibitor, comprising determining the subject's CDX2 expression level, and/or the expression level of one or more surrogate biomarkers set forth in Table 1 or Table 2, in a biological sample obtained from the subject, wherein a CDX2 positive expression level, and/or a positive expression level of a surrogate biomarker set forth in Table 1 or Table 2, indicates that the subject is likely to be responsive to treatment with an EGFR inhibitor, and a negative CDX2 expression levels, and/or a negative expression level of a surrogate biomarker set forth in Table 1 or Table 2 indicates that the subject is not likely to be responsive to treatment with an EGFR inhibitor.

In another embodiment, the present invention provides a method for predicting whether a subject diagnosed with colorectal cancer who is KRAS^{wild-type}, NRAS^{wild-type} with a left-side originating colon tumor, is likely to be responsive to treatment with an EGFR inhibitor, comprising determining the subject's CDX2 expression level, and/or the expression level of one or more surrogate biomarkers set forth in Table 1 or Table 2, in a biological sample obtained from the subject, wherein a CDX2 positive expression level, and/or a positive expression level of a surrogate biomarker set forth in Table 1 or Table 2, indicates that the subject is likely to be responsive to treatment with an EGFR inhibitor, and a negative CDX2 expression levels, and/or a negative expression level of a surrogate biomarker set forth in Table 1 or Table 2 indicates that the subject is not likely to be responsive to treatment with an EGFR inhibitor.

In one embodiment, a colon tumor originating on the right side originated from the caecum, the ascending colon or the transverse colon up to the splenic flexure. In another embodiment, a colon tumor that originated on the left side originated from the descending colon distal to the splenic flexure, the sigmoid colon or the rectum.

In one embodiment, the methods of the invention further comprise determining whether a patient with one or more mutations in the BRAF gene would benefit from therapy with a BRAF inhibitor and/or a MEK inhibitor and/or an ERK inhibitor, and/or an EGFR inhibitor, wherein the therapy with a BRAF inhibitor and/or a MEK inhibitor and/or an ERK inhibitor, and/or an EGFR inhibitor is selected when the subject's CDX2 expression level, and/or the expression level of one or more surrogate biomarkers set forth in Table 1 or Table 2, is positive, and the therapy with a BRAF inhibitor and/or a MEK inhibitor and/or an ERK inhibitor, and/or an EGFR inhibitor is not selected when the subject's CDX2 expression level and/or the expression level of one or more surrogate biomarkers set forth in Table 1 or Table 2, is negative. In one embodiment, the BRAF inhibitor is vemurafenib or dabrafenib. In another embodiment, the MEK inhibitor is trametinib or selumetinib. In another embodiment, the ERK inhibitors is SCH772984 or VTX11e.

In another embodiment, the methods of the invention further comprise determining whether a patient with one or more mutations in the BRAF gene would benefit from therapy with a BRAF inhibitor, a MEK inhibitor and/or an EGFR inhibitor, wherein the therapy is selected when the subject's CDX2 expression level, and/or the expression level of one or more surrogate biomarkers set forth in Table 1 or Table 2, is positive, and the therapy is not selected when the subject's CDX2 expression level, and/or the expression level of one or more surrogate biomarkers set forth in Table 1 or Table 2, is CDX2 negative. In one embodiment, the MEK inhibitor is trametinib or selumetinib.

### Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein, the term “control” refers to any entity used in comparison of biomarker expression. For example, in one embodiment, a control can be the expression pattern of the biomarkers in an individual not affected by the disease. In another embodiment, a control can be the averaged expression pattern of the biomarkers from a group or population of individuals not affected by the disease. In another embodiment, a control can be the expression of another gene/protein in the same individual. In another embodiment, a control can be a threshold on the score produced by a mathematical model that uses the expressions of biomarkers and possibly expression of other genes/proteins so that scores for disease-affected individuals and for individuals not affected by the disease significantly differ. The expression and the expression pattern can be either absolute or relative, i.e., determined relative to the expression of some other gene(s)/protein(s). In specific embodiments, the control is derived at least in part from the level of expression of one or more reference genes or proteins from a single individual without colorectal cancer. In another embodiment, the control is derived at least in part from the level of expression of one or more reference genes or proteins from a population of individuals without colorectal cancer, e.g., the average level of expression. One of skill in the art recognizes that the control expression level may be normalized by standard means in the art. The normalization may include standardization to a reference protein (such as a housekeeping gene including GAPDH), for example.

As used herein, the term “biological sample” refers to a sample of biological material obtained from a subject, preferably a human subject, including a tissue sample, e.g., a colorectal tumor tissue sample (such as a primary tumor sample or a metastatic tumor sample), a cell sample, e.g., isolated tumor cells, or a biological fluid, e.g., blood (including serum or plasma).

The term “patient” or “subject,” as used interchangeably herein, refers to any warm-blooded animal, preferably a human.

The term “tumor,” as used herein, refers to any neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include, but are not limited to, colorectal cancer, breast cancer, ovarian cancer, lung cancer, prostate cancer, hepatocellular cancer, gastric cancer, pancreatic cancer, cervical cancer, liver cancer, bladder cancer, cancer of the urinary tract, thyroid cancer, renal cancer, carcinoma, melanoma, and brain cancer. In one embodiment, the cancer is a cancer in which the signaling pathway through EGFR is involved. In another embodiment the cancer is colorectal cancer.

The terms “colorectal cancer” or “CRC”, used interchangeably herein, are used in the broadest sense and refer to (1) all stages and all forms of cancer arising from epithelial cells of the intestinal tract below the small intestine (i.e., the large intestine (colon), including the cecum, ascending colon, transverse colon, descending colon, and sigmoid colon, and rectum), and/or (2) all stages and all forms of cancer affecting the lining of the large intestine and/or rectum. In the staging systems used for classification of colorectal cancer, the colon and rectum are treated as one organ. Additionally, as used herein, the term “colorectal cancer” further includes medical conditions which are characterized by cancer of cells of the duodenum and small intestine (jejunum and ileum).

CRC can originate from the right side or the left side of the colon. In one embodiment, the right side comprises the caecum, the ascending colon and the transverse colon up to the splenic flexure. In another embodiment, the left side comprises the descending colon distal to the splenic flexure, the sigmoid colon and the rectum. Assessment of origination of CRC can be carried out by methods known to those skilled in the art.

CRC may be staged according to the Dukes system, the Astler-Coller system or the TNM system (tumors/nodes/metastases), whereby the latter is most commonly used. The TNM system of the American Joint Committee of Cancer (AJCC) describes the size of the primary tumor (T), the degree of lymph node involvement (N) and whether the cancer has already formed distant metastasis (M), i.e., spread to other parts of the body. Here, stages 0, IA, IB, IIA, IIB, III and IV are defined based on the determined T-, N- and M-values. A corresponding staging scheme can be derived from the Cancer Staging Manual of the AJCC (Edge et al., 2010 Ann Surg. Oncol. June; 17(6):1471-4). Another system for staging of colorectal cancer is the Dukes system established by the British pathologist Cuthbert Dukes, defining cancer stages A, B, C and D. This system was adapted by Astler and Coller, who further subdivided stages B and C (“modified Astler-Coller classification”). As used herein, a CRC patient includes patients staged according to any staging system used and irrespective of the stage diagnosed.

As used herein, “a patient suffering from colorectal cancer” refers to any mammalian, in particular human, patient having developed atypical and/or malignant cells in the lining and/or the epithelium of the large intestine and/or rectum. This includes CRC patients independent of the stage and form of the CRC. Patients suffering from colorectal cancer also include patients which are recurrent with colorectal cancer, i.e., patients wherein after surgical treatment the tumor could no longer be detected for a certain time span, but wherein the cancer has returned in the same or different part of the large intestine, and/or rectum and/or wherein metastases have developed at different sites of the patient's body such as in the liver, lung, peritoneum, lymph nodes, brain and/or bones. In another embodiment, the patient suffering from CRC is a patient wherein the initial tumor has already been treated surgically and the CRC is non-metastatic.

The term “prediction” or “predicting” is used herein to refer to the likelihood that a patient will have a particular clinical outcome, whether positive or negative, following treatment with an EGFR inhibitor. The predictive methods of the present disclosure can be used clinically to make treatment decisions by choosing the most appropriate treatment modalities for any particular patient. The predictive methods of the present disclosure are valuable tools in predicting if a patient is likely to be responsive or non-responsive to a treatment regimen, such as a treatment regimen including an EGFR inhibitor, alone or in combination with another cancer treatment.

Whether a patient or a tumor is “responsive,” as used herein with respect to a clinical response to treatment, such as treatment with an EGFR inhibitor, can be assessed using any endpoint indicating a benefit to the patient, including, without limitation, (1) inhibition, to some extent, of tumor growth, including slowing down and complete growth arrest; (2) reduction in the number of tumor cells; (3) reduction or shrinkage in tumor size; (4) inhibition (i.e., reduction, slowing down or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Responsiveness may also be expressed in terms of various measures of clinical outcome. Positive clinical outcome can also be considered in the context of an individual's outcome relative to an outcome of a population of patients having a comparable clinical diagnosis. In one embodiment, an increase in the likelihood of positive clinical response corresponds to a decrease in the likelihood of cancer recurrence.

In another embodiment, clinical response to treatment can be measured based on disease control (DC), wherein tumors displaying disease control include tumors whose response to treatment is a complete response (CR), partial response (PR) or stable disease (SD). In one embodiment, tumors displaying disease control do not include tumors in a progressive disease (PD) state.

In another embodiment, clinical response to treatment can be measured based on an objective tumor response, e.g., tumor shrinkage, wherein tumors undergoing an objective tumor response include tumors undergoing either a complete response (CR) or a partial response (PR). In one embodiment, tumors undergoing an objective tumor response do not include tumors that display stable disease (SD) or tumors in a progressive disease (PD) state.

“Non-responsive” “resistant” or “refractory” as used interchangeably herein with respect to a clinical response to treatment, such as treatment with an EGFR inhibitor, refers to cancer that does not respond to the treatment. The lack of response can be assessed by, for example, lack of inhibition of tumor growth or increased tumor growth; lack of reduction in the number of tumor cells or an increase in the number of tumor cells; increased tumor cell infiltration into adjacent peripheral organs and/or tissues; increased metastasis; decrease in the length of survival following treatment; and/or mortality. The cancer may be resistant at the beginning of treatment or it may become resistant during treatment.

Metrics or endpoints that can be used to assess responsiveness or non-responsiveness to treatment, such as treatment with an EGFR inhibitor, include, but are not limited to Recurrence-Free interval (RFI), Overall Survival (OS), Disease-Free Survival (DFS), Distant Recurrence-Free Interval (DRFI), progression-free survival (PFS), relapse-free survival (RFS), disease-specific survival (DSS), cancer-specific survival (CSS), time-to-recurrence (TTR), time-to-treatment-failure (TTF), quality-adjusted life years (QALY), and the like. Exemplary metrics are described in Punt et al., J Natl Cancer Inst, 99:998-1003, 2007, the contents of which are expressly incorporated by reference herein.

The term “microarray” refers to an ordered arrangement of hybridizable array elements, such as polynucleotide probes, on a substrate.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein (including the CDX2 polynucleotide) include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The terms “level of expression of a gene”, “gene expression level”, “level of a marker”, and the like refer to the level of mRNA, as well as pre-mRNA nascent transcript(s), transcript processing intermediates, mature mRNA(s) and degradation products, or the level of protein, encoded by the gene in the cell.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature.

The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

In the context of the present disclosure, reference to “at least one,” “at least two,” “at least five,” etc. of the markers listed in any particular marker set, e.g., CDX2, KRAS, NRAS, BRAF, EGFR and/or PIK3CA, or a surrogate CDX2 biomarker, means any one or any and all combinations of the markers listed.

Reference to markers for “prediction of response to EGFR inhibitors”, and like expressions, encompass within their meaning response to treatment comprising an EGFR inhibitor as monotherapy, or in combination with other agents, or as prodrugs, or together with local therapies such as surgery and radiation, or as adjuvant or neoadjuvant chemotherapy, or as part of a multimodal approach to the treatment of neoplastic disease.

An anti-EGFR combination or anti-EGFR combination therapy refers to a combination of an EGFR inhibitor and another agent. A number of agents can be combined with EGFR inhibitor to enhance the cytotoxic activity through biochemical modulation. Such combination therapies include, but are not limited to, the use of other cancer chemotherapeutics, radiation, and surgery, and other cancer therapeutics known in the art and disclosed herein.

As used herein, the terms “EGFR inhibitor” and “anti-EGFR” are used interchangeably throughout, and encompass an agent with EGFR inhibitory activity or a prodrug thereof, and further encompass an anti-EGFR combination therapy (e.g., an anti-EGFR with the one or more of the agents exemplified herein or known in the art). In one embodiment, an EGFR inhibitor includes an anti-EGFR antibody such as, for example, cetuximab, panitumumab, necitumumab, zalutumumab, nimotuzumab and matuzumab. In another embodiment, an EGFR inhibitor comprises a combination or mixture of multiple anti-EGFR monoclonal antibodies, either directed against the same or different epitopes of the EGFR molecule, for example as described by Arena et al., Science Translational Medicine, 8:324ra14, 2016, the contents of which are hereby incorporated by reference. In another embodiment, an EGFR inhibitor is a small molecule.

The term “antibody,” as used herein, refers to an intact antibody, or a binding fragment thereof that competes with the intact antibody for specific binding and includes chimeric, humanized, fully human, and bispecific antibodies. In certain embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)2, Fv, immunologically functional immunoglobulin fragments, heavy chain, light chain, and single-chain antibodies.

As used herein, the term “biomarker” or “marker” refers to both a marker (e.g., an expressed gene, including mRNA and/or protein) or a panel of markers, that allows prediction of whether a carcinoma, e.g., a colorectal carcinoma is likely to be resistant to a particular therapeutic, e.g., an EGFR inhibitor. A “biomarker nucleic acid” is a nucleic acid (e.g., mRNA, cDNA) encoded by or corresponding to a biomarker of the invention. Such biomarker nucleic acids include DNA (e.g., cDNA) comprising the entire or a partial sequence of a nucleic acid sequence provided herein or known in the art, or the complement of such a sequence. The marker nucleic acids also include RNA comprising the entire or a partial sequence of a nucleic acid sequence provided herein or known in the art, or the complement of such a sequence, wherein all thymidine residues are replaced with uridine residues. A “marker protein” is a protein encoded by or corresponding to a marker of the invention. A marker protein comprises the entire or a partial sequence of an amino acid sequence provided herein or known in the art. The terms “protein” and “polypeptide” are used interchangeably.

As used herein, the term “CDX2” or “caudal type homeobox 2” is a member of the caudal-related homeobox transcription factor gene family. CDX2 is a major regulator of intestine-specific genes involved in cell growth and differentiation. This protein also plays a role in the early embryonic development of the gastro-intestinal tract. Aberrant expression of this gene is associated with intestinal inflammation and tumorigenesis. As used herein, CDX2 refers to both the gene and the protein unless clearly indicated otherwise by context. Exemplary, non-limiting National Center for Biotechnology Information (NCBI) Accession Numbers for CDX2 human mRNA and protein are: GenBank U51096 (SEQ ID NO: 1) and RefSeq NP_001256.3 (SEQ ID NO: 2), respectively. The nucleotide sequence encoding human CDX2 protein is disclosed in Mallo, G. V. et al. 1997 Intl. J. Cancer 74(1):35-44, the contents of which are expressly incorporated herein by reference. It is understood that the invention includes the use of any fragments of CDX2 sequences as long as the fragment can allow for the specific identification of CDX2. Moreover, it is understood that there are naturally occurring variants of CDX2 which may or may not be associated with a specific disease state, the use of which are also included in this application. Exemplary, non-limiting NCBI Accession Numbers for CDX2 human mRNA and protein sequences bearing representative single nucleotide polymorphisms (SNPs) include GenBank BC014461 and RefSeq NM_001265.4. The sequence of exemplary, non-limiting SNPs in the human CDX2 gene is reported in Sivagnanasundaram et al., British Journal of Cancer, 84:218-225, 2001; Rozek et al., Cancer Research, 65:5488-92, 2005.

As used herein, the determining the “mutation status” of KRAS, BRAF, NRAS, EGFR or PIK3CA, refers to the determination of the presence or absence of one or more mutations in KRAS, BRAF, NRAS, EGFR or PIK3CA associated with responsiveness or non-responsiveness to treatment with an EGFR inhibitor. The mutation status of a gene or protein can be determined by any means known in the art.

As used herein, the terms “KRAS mutation”, “BRAF mutation”, “NRAS mutation”, “EGFR mutation” or “PIK3CA mutation” include any one or more mutations recognized in the art as associated with responsiveness or non-responsiveness to treatment with an EGFR inhibitor. In one embodiment, mutations in each of KRAS, NRAS, BRAF, EGFR and PIK3CA include, but are not limited to, those mutations forth in Zhang et al. Scientific Reports 5:18678, 2015, in Yao et al., Nature, 548:234-238, 2017, and in Arena et al., Clinical Cancer Research, 21:2157-2166, 2015, the contents of which are hereby incorporated herein by reference.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to.”

The term “or” is used inclusively herein to mean, and is used interchangeably with, the term “and/or,” unless context clearly indicates otherwise.

The term “such as” is used herein to mean, and is used interchangeably, with the phrase “such as but not limited to.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “one or more” is understood as each value 1, 2, 3, 4, 5, 6, 7, 8, 9, 10, and any value greater than 10.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### Predictive Methods of the Invention

Based on the lack of expression of CDX2, alone or in combination with a surrogate biomarker of CDX2 (e.g., as detected by assaying for an RNA transcript or expression product thereof) in cancer cells that are refractory to treatment with an EGFR inhibitor, the present disclosure provides predictive markers for responsiveness of colorectal cancer to an EGFR inhibitor. The predictive markers and associated information provided by the present disclosure allow physicians to make more intelligent treatment decisions, and to customize the treatment of colorectal cancer to the needs of individual patients, thereby maximizing the benefit of treatment and minimizing the exposure of patients to unnecessary treatments, which do not provide any significant benefits and often carry serious risks due to toxic side-effects.

In one particular embodiment, a method for predicting whether a subject diagnosed with colorectal cancer is likely to be responsive or non-responsive (refractory) to treatment with an EGFR inhibitor is disclosed. In certain embodiments, the method includes determining the subject's CDX2 expression level, and/or the expression level of one or more surrogate biomarkers set forth in Table 1 or Table 2, in a biological sample obtained from the subject. In a specific embodiment, a CDX2 positive expression level, and/or a positive expression level of one or more surrogate biomarkers set forth in Table 1 or Table 2, indicates that the subject is likely to be responsive to treatment with an EGFR inhibitor and a CDX2 negative expression level, and/or a negative expression level of one or more surrogate biomarkers set forth in Table 1 or Table 2, indicates that the subject is likely to be non-responsive or refractory to treatment with an EGFR inhibitor.

In another embodiment, a method of assessing the efficacy of a therapeutic agent for treating colorectal cancer in a subject prior to administration of the therapeutic agent is disclosed. In certain embodiments, the method includes determining the subject's CDX2 expression level and/or the expression level of one or more surrogate biomarkers set forth in Table 1 or Table 2, in a biological sample obtained from the subject, and predicting that the therapeutic agent will be efficacious for treating colorectal cancer when the subject's CDX2 expression level is CDX2 positive, and/or the expression level of one or more surrogate biomarkers set forth in Table 1 or Table 2 is positive, and non-efficacious for treating colorectal cancer with the subject's CDX2 expression level is CDX2 negative, and/or the expression level of one or more surrogate biomarkers set forth in Table 1 or Table 2 is negative.

In yet another embodiment, a method for selecting a subject diagnosed with colorectal cancer for treatment with an EGFR inhibitor is disclosed. In certain embodiments, the method includes determining the subject's CDX2 expression level, and/or the expression level of one or more surrogate biomarkers set forth in Table 1 or Table 2, in a biological sample obtained from the subject, and selecting a subject for treatment with an EGFR inhibitor if the subject has a CDX2 positive expression level, and/or the expression level of one or more surrogate biomarkers set forth in Table 1 or Table 2 is positive

In another embodiment, a method of determining a clinical course of therapy for treating colorectal cancer in a subject is disclosed. In certain embodiments, the method includes determining the subject's CDX2 expression level, and/or the expression level of one or more surrogate biomarkers set forth in Table 1 or Table 2, in a biological sample obtained from the subject, and identifying a clinical course of therapy based on the subject's CDX2 expression level and/or the expression level of the one or more surrogate biomarkers set forth in Table 1 or Table 2. In a specific embodiment, therapy with an EGFR inhibitor is selected when the subject's CDX2 expression level is CDX2 positive, and/or where the expression level of one or more surrogate biomarkers set forth in Table 1 or Table 2 is positive.

In still another embodiment, a method of treating colorectal cancer in a subject is disclosed. In certain embodiments, the method includes determining the subject's CDX2 expression level, and/or the expression level of one or more surrogate biomarkers set forth in Table 1 or Table 2, in a biological sample obtained from the subject, and administering an EGFR inhibitor when the subject's CDX2 expression level is CDX2 positive, and/or where the expression level of one or more surrogate biomarkers set forth in Table 1 or Table 2 is positive.

In some embodiments, the methods of the invention further comprise assessing whether the CRC originated on the right side or the left side of the colon. In one embodiment, the CRC originated on the right side. In another embodiment, the CRC originated on the left side.

In one embodiment, one or more additional anti-cancer therapeutic agents can be administered to the patient (either sequentially or concurrently), in addition to an EGFR inhibitor, including, but not limited, to chemotherapy or radiation. Exemplary additional anti-cancer agents include, but are not limited to: 1) alkylating agents, including, but not limited to: a] nitrogen mustards, including, but not limited to mechlorethamine, cyclophosphamide, ifosfamide, chlorambucil, melphalan, busulfan, alone or in combination with sodium 2-sulfanylethanesulfonate (Mesna); b] nitrosoureas, including, but not limited to N-Nitroso-N-methylurea (MNU), carmustine (BCNU), lomustine (CCNU) and semustine (MeCCNU), fotemustine and streptozotocin; c] tetrazines, including, but not limited to dacarbazine, mitozolomide, and temozolomide; d] aziridines, including, but not limited to thiotepa, mytomycin and diaziquone (AZQ); e] platinum coordination compounds, including, but not limited to cisplatin, carboplatin, oxaliplatin; f] other alkylating agents, including, but not limited to procarbazine, and hexamethylmelamine; 2) anti-metabolites, including, but not limited to: a] inhibitors of dyhydrofolate reductase (DHFR) as well as inhibitors of other enzymes involved in folate metabolism, including, but not limited to methotrexate, pemetrexed and raltritrexed; b] fluoropyrimidines as well as other inhibitors of thymidylate synthase (TS), including, but not limited to 5-fluorouracil alone or in combination with leucovorin, capecitabine, floxuridine, tegafur (UFT, UFUR) and trifluridine alone or in combination with inhibitors of thymidine phosphorylase, such as tipiracil; c] deoxynucleoside analogues including, but not limited to cytarabine, gemcitabine, decitabine, fludarabine, nelarabine, cladribine, clofarabine and pentostatin; d] ribonucleoside analogues including, but not limited to azacitidine, e] thiopurines including, but not limited to thioguanine and mercaptopurine; 3) inhibitors of the microtubule function, including, but not limited to: a] vinca alkaloids, including, but not limited to vincristine, vinblastine, vindesine and vinorelbine; b] taxanes including, but not limited to paclitaxel, docetaxel and cabazitaxel; c] analogs of epothilone B, including, but not limited to ixabepilone; 4) inhibitors of topoisomerase, including, but not limited to: a] inhibitors of topoisomerase I, including, but not limited to irinotecan and topotecan; b] inhibitors of topoisomerase II, including, but not limited to etoposide, teniposide, doxorubicin, novobiocin, bleomycin, merbarone and mitoxantrone; and 5) cytotoxic antibiotics, including, but not limited to: a] anthracyclins, including, but not limited to doxorubicin (adriamycin), daunorubicin, idarubicin, epirubicin, pirarubicin, iododoxorubicin, nemorubicin, and aclarubicin, either alone or in liposomal formulations; b] other cytotoxic antibiotics, including, but not limited to bleomycin, mitomycin C, mitoxantrone, actinomycin; 6) other miscellaneous cytotoxic compounds, including, but not limited to trabectedin (ecteinascidin 743, ET-743); 7) inhibitors of angiogenesis, including, but not limited to: a] anti-VEGF monoclonal antibodies, including, but not limited to bevacizumab; b] anti-VEGFR monoclonal antibodies, including, but not limited to ramucirumab; c] recombinant, chimeric, soluble and/or re-engineered versions of VEGFR, including, but not limited to aflibercept; d] inhibitors of VEGFR tyrosine kinase activity, including, but not limited to regorafenib, sorafenib, pazopanib, and sunitinib; 8) immune check-point inhibitors, including, but not limited to: a] anti-CTLA4 monoclonal antibodies, including, but not limited to ipilimumab and tremelimumab; b] anti-PD1 monoclonal antibodies, including, but not limited to nivolumab and pembrolizumab; c] anti-PDL1 monoclonal antibodies, including, but not limited to atezolizumab; 9) inhibitors of HER2, including, but not limited to; a] anti-HER2 monoclonal antibodies, including, but not limited to trastuzumab and pertuzumab, either alone or in combination (e.g. trastuzumab+pertuzumab) or conjugated to cytotoxins or radionucliudes; b] inhibitors of HER2 tyrosine kinase activity, including, but not limited to lapatinib; 11) anti-RANKL monoclonal antibodies, including, but not limited to denosumab; 12) inhibitors of BRAF tyrosine kinase activity, including, but not limited to vemurafenib, and dabrafenib; 13) inhibitors of MEK tyrosine kinase activity, including, but not limited to trametinib and selumetinib; 14) inhibitors of ALK tyrosine kinase activity, including, but not limited to crizotinib; 15) inhibitors of MET tyrosine kinase activity, including, but not limited to cabozantinib; 16) inhibitors of KIT tyrosine kinase activity, including, but not limited to imatinib and dasatinib; 17) inhibitors of ABL tyrosine kinase activity, including, but not limited to imatinib, dasatinib, nilotinib and ponatinib; 18) inhibitors of CDK tyrosine kinase activity, including, but not limited to palbociclib and amenaciclib; 19) inhibitors of COX1 enzymes, including, but not limited to acetyl-salicylic acid, naproxene, ibuprofen, indomethacyn, and diclofenac; 20) inhibitors of COX2 enzymes, including, but not limited to celecoxib and rofecoxib; 21) inhibitors of PARP enzymes, including, but not limited to olaparib, niraparib and veliparib; and 22) others.

In another embodiment, where the subject is either CDX2 positive or CDX2 negative (and/or positive or negative for one or more of the surrogate biomarkers), an EGFR inhibitor is administered with at least one additional therapeutic agent (either sequentially or concurrently), wherein the additional therapeutic agent is capable of disabling the tumor resistance mechanisms, and thus restoring the tumor's sensitivity to the EGFR inhibitor.

In one embodiment, higher levels of CDX2, and/or a surrogate biomarker, is positively correlated with a higher probability of response to treatment with an EGFR inhibitor (i.e., higher levels of CDX2, and/or a surrogate biomarker, is correlated with increased responsiveness to treatment).

The predictive markers and associated information provided by the present disclosure predicting the clinical outcome of treatment with an EGFR inhibitor of colorectal cancer also have utility in screening patients for inclusion in clinical trials that test the efficacy of other drug compounds. The predictive markers and associated information provided by the present disclosure predicting the clinical outcome of treatment with an EGFR inhibitor of CRC are useful as inclusion criterion for a clinical trial. For example, a patient is more likely to be included in a clinical trial for an EGFR inhibitor if the results of the test indicate that the patient will have a good clinical outcome if treated with an EGFR inhibitor; and a patient is less likely to be included in a clinical trial if the results of the test indicate that the patient will have a poor clinical outcome if treated with an EGFR inhibitor.

In one embodiment, the primary biomarker used in the methods of the present invention is CDX2. In certain, non-limiting embodiments, additional biomarkers may be used in the disclosed methods. In one embodiment, one or more of the surrogate biomarkers set forth in Table 1 and Table 2 is used in the methods of the invention, alone or in combination with CDX2. In one embodiment, CDX2 is used in the methods of the invention in combination with one or more surrogate biomarker. In another embodiment, one or more surrogate biomarker is used in the methods of the invention.

In another specific embodiment, additional biomarkers used in the methods of the invention include, but are not limited to, one or more biomarkers selected from the group consisting of KRAS, NRAS, BRAF, EGFR and PIK3CA, and combinations thereof. In other embodiments, the methods of the invention further comprise genotyping for the presence or absence of one or more mutant alleles (e.g., somatic mutations) in genes such as KRAS, NRAS, BRAF, EGFR and/or PIK3CA (e.g., at one, two, three, four, five, or more polymorphic sites such as a SNP in one or more of these genes) in a sample obtained from a subject, e.g., a tumor tissue or cell sample or a serum sample. In particular embodiments, the determination of the positive or negative expression of CDX2 in conjunction with the determination of the mutation status of one or more additional biomarkers further aids or improves the selection of a suitable anticancer drug and/or the identification or prediction of a response thereto in cells such as colorectal cancer cells (e.g., isolated cancer cells from a colorectal tumor). In one embodiment, the mutation status of at least KRAS is determined in addition to the expression level of CDX2 or a CDX2 surrogate biomarker. In another embodiment, the mutation status of KRAS and one or more of BRAF and NRAS is determined in addition to the expression level of CDX2 or a CDX2 surrogate biomarker. In another embodiment, the mutation status of KRAS and one or more of NRAS, BRAF, EGFR and PIK3CA is determined in addition to the expression level of CDX2, or a CDX2 surrogate biomarker.

In one embodiment, the present invention provides methods for determining whether a patient with or without one or more mutations in the BRAF gene would benefit from therapy with an EGFR inhibitor alone or in combination with a BRAF inhibitor. In one embodiment, therapy with a BRAF inhibitor is selected when the subject's CDX2 expression level is CDX2 positive, and/or when the expression level of one or more surrogate biomarkers set forth in Table 1 or Table 2 is positive. In another embodiment, therapy with a BRAF inhibitor is not selected when the subject's CDX2 expression level is CDX2 negative, and/or when the expression level of one or more surrogate biomarkers set forth in Table 1 or Table 2 is negative. In one embodiment, the BRAF inhibitor is vemurafenib or dabrafenib.

In another embodiment, the present invention provides methods for determining whether a patient with or without one or more mutations in the KRAS, NRAS, BRAF, EGFR or PIK3CA genes would benefit from therapy with an EGFR inhibitor in combination with a MEK inhibitor. In one embodiment, therapy with a MEK inhibitor is selected when the subject's CDX2 expression level is CDX2 positive, and/or when the expression level of one or more surrogate biomarkers set forth in Table 1 or Table 2 is positive. In another embodiment, therapy with a MEK inhibitor is not selected when the subject's CDX2 expression level is CDX2 negative, and/or when the expression level of one or more surrogate biomarkers set forth in Table 1 or Table 2 is negative. In one embodiment, the MEK inhibitor is trametinib or selumetinib.

In one embodiment, the present invention also provides methods of determining whether a patient with or without one or more mutations in the KRAS, NRAS, BRAF, EGFR or PIK3CA genes would benefit from therapy with an EGFR inhibitor used in combination with one or more molecules that are considered to be surrogates of EGFR inhibitors, or synergistic with EGFR inhibitors. Surrogates of EGFR inhibitors or inhibitors that are synergistic with an EGFR inhibitor are able to inhibit signaling molecules that can be activated directly or indirectly by the EGFR signaling pathway (i.e., signaling molecules that are “downstream” of the EGFR signaling pathway). Thus, one embodiment, therapy with an EGFR inhibitor in combination with one or more molecules that are surrogates of EGFR inhibitors or synergistic with EGFR inhibitors is selected when the subject's CDX2 expression level is CDX2 positive, and/or when the expression level of one or more surrogate biomarkers set forth in Table 1 or Table 2 is positive. In another embodiment, therapy with an EGFR inhibitor in combination with one or more molecules that are surrogates of EGFR inhibitors, or synergistic with EGFR inhibitors is not selected when the subject's CDX2 expression level is CDX2 negative, and/or when the expression level of one or more surrogate biomarkers set forth in Table 1 or Table 2 is negative.

In one embodiment, synergistic or surrogate molecules comprise BRAF inhibitors (e.g. vemurafenib, dabrafenib), MEK inhibitors (e.g. trametinib or selumetinib), and ERK inhibitors (e.g. SCH772984, VTX11e). Additional examples of clinically approved and/or investigational BRAF, MEK and ERK inhibitors are set forth in Samatar and Poulikakos, Nature Reviews in Drug Discovery, 13:928-942, 2014.

In another embodiment, the present invention also comprises methods for determining whether a patient with or without one or more mutations in the KRAS, NRAS, BRAF, EGFR or PIK3CA genes would benefit from therapy with an EGFR inhibitor in combination with an ERK inhibitor. In one embodiment, the therapy with an EGFR inhibitor in combination with an ERK inhibitor is selected when the subject's CDX2 expression level is CDX2 positive, and/or when the expression level of one or more surrogate biomarkers set forth in Table 1 or Table 2 is positive. In another embodiment, the therapy with an EGFR inhibitor in combination with an ERK inhibitor is not selected when the subject's CDX2 expression level is CDX2 negative, and/or when the expression level of one or more surrogate biomarkers set forth in Table 1 or Table 2 is negative. In one embodiment, the ERK inhibitor is SCH772984 or VTX11e.

In some embodiments, the method further comprises determining whether a patient with or without one or more mutations in the KRAS, NRAS, BRAF, EGFR or PIK3CA genes would benefit from therapy with combinations of multiple synergistic inhibitors of the EGFR signaling pathway and its downstream targets. In one embodiment, therapy with synergistic inhibitors of the EGFR signaling pathway is selected when the subject's CDX2 expression level is CDX2 positive, and/or when the expression level of one or more surrogate biomarkers set forth in Table 1 or Table 2 is positive. In another embodiment, therapy with synergistic inhibitors of the EGFR signaling pathway is not selected when the subject's CDX2 expression level is CDX2 negative, and/or when the expression level of one or more surrogate biomarkers set forth in Table 1 or Table 2 is negative.

In one embodiment, synergistic inhibitors of the EGFR signaling pathway and its downstream targets comprise, for example, EGFR inhibitors, BRAF inhibitors (e.g., vemurafenib, dabrafenib), MEK inhibitors (e.g., trametinib or selumetinib) and ERK inhibitors (e.g., SCH772984, VTX11e). In another embodiment, the combination of EGFR inhibitors, BRAF inhibitors (e.g., vemurafenib, dabrafenib), MEK inhibitors (e.g., trametinib or selumetinib) and ERK inhibitors (e.g., SCH772984, VTX11e) include those set forth in Samatar and Poulikakos, Nature Reviews in Drug Discovery, 13:928-942, 2014; Corcoran, Journal of Gastrointestinal Oncology, 6:650-659, 2015; Xue et al., Nature Medicine, 23:929-937, 2017; Kirouac et al., NPJ Systems Biology and Applications, 3:14, 2017.

In one embodiment, the biological sample is a colorectal tumor sample, e.g., obtained from a tissue biopsy. In some embodiments, the tumor sample is a fixed, paraffin-embedded tissue sample. In another embodiment, the sample is a blood sample, e.g., a serum sample.

### CDX2 Expression Levels

In certain non-limiting embodiments, a subject's CDX2 expression level is defined as “CDX2 negative” or “CDX2 positive.” In one specific embodiment, a “CDX2 negative expression level” (or CDX2^{neg}) is defined as either a complete lack of CDX2 expression or a “low” level of CDX2 expression (i.e., CDX2^{neg/low}), while a “CDX2 positive expression level” (or CDX2^{pos}) is defined as a “high” level of CDX2 expression (i.e., CDX2^{high}).

In some embodiments, the method used to determine the subject's CDX2 expression level includes the evaluation of CDX2 expression levels in individual cancer cells within the subject's tumor, or a sample thereof, and the calculation of the percentage of CDX2 positive cancer cells and CDX2 negative cancer cells. A subject may be determined to be CDX2 positive if a percentage of cancer cells in the subject's tumor, or a sample thereof, is above a predetermined threshold. A subject may be determined to be CDX2 negative if a percentage of cancer cells in the subject's tumor, or a sample thereof, is below a predetermined threshold. In one embodiment, the threshold can be determined using the methods disclosed herein.

In one exemplary embodiment, the subject is determined to be CDX2 negative if 0%, 5%, 10%, 15%, 20%, 25%, 30%, 35%, 40%, or 45% of the subject's cancer cells in the subject's tumor, or a sample thereof, are negative for CDX2. In another embodiment, the subject is determined to be CDX2 positive if 50%, 55%, 60%, 65%, 70%, 75%, 80%, 85%, 90%, 95%, or 100% of the subject's cancer cells in the subject's tumor, or a sample thereof, are positive for CDX2.

In certain non-limiting embodiments, a CDX2^{pos} expression level is defined as a CDX2 expression level that is higher than that of a specific threshold which separates “low” from “high” CDX2 expression levels. For example, a CDX2^{pos} expression level may be defined as a CDX2 expression level that is greater than (e.g., 5%, 10%, 15%, 20%, 25%, 30%, 35%, 40%, 45%, 50%, 55%, 60%, 65%, 70%, 75%, 80%, 85%, 90%, 95%, or 100% greater than) or equal to that of a specific threshold identified as separating “low” from “high” CDX2 expression levels. A negative CDX2 expression level may be defined as a CDX2 expression level that is less than (e.g., 5%, 10%, 15%, 20%, 25%, 30%, 35%, 40%, 45%, 50%, 55%, 60%, 65%, 70%, 75%, 80%, 85%, 90%, 95%, or 100% less than) or equal to a threshold level chosen to separate samples with low levels of CDX2 expression (CDX2^{low}) from samples with high levels of CDX2 expression (CDX2^{high}). In one embodiment, the threshold separating CDX2^{neg} from CDX2^{pos} tumors may be identified based on the assumption that CDX2^{neg} and CDX2^{pos} tumors represent two distinct populations of samples, and that CDX2 expression levels are distributed according to a bimodal distribution (Dalerba et al., NEJM, 374:211-222, 2016; Bae et al., World J. Gastroenterol., 21:1457-1467, 2015).

The CDX2 expression level used as the threshold to separate CDX2^{neg} from CDX2^{pos} tumors may be chosen mathematically. For example, the threshold expression value may be the CDX2 expression value that represents the lowest frequency among the CDX2 expression values found between the two modes of the bimodal distribution of the CDX2 expression values. In one embodiment, the mathematical approach assumes a bimodal distribution of the CDX2 expression values. In another embodiment, the mathematical approach used to determine the threshold value is the StepMiner algorithm. (Sahoo et al., Nucleic Acids Research, 35:3705-3712, 2007). The StepMiner algorithm is incorporated into the BooleanNet software (Sahoo et al., Genome Biology, 9:R157, 2008) and can be used to stratify human colon carcinomas into binary subgroups (“neg/low” or CDX2^{neg} vs. “pos/high” or CDX2^{pos}) based on the expression levels of many individual genes (Dalerba et al., Nature Biotechnology, 29:1120-1127, 2011).

Alternatively, in another embodiment, the CDX2 expression level used as the threshold to separate CDX2^{neg} from CDX2^{pos} tumors may be chosen empirically. For example, the CDX2 expression value may be the value below which no clinical benefit can be observed as a result of treatment with anti-EGFR monoclonal antibodies (e.g., cetuximab, panitumumab). Accordingly, in a specific embodiment, the CDX2 expression level chosen as the threshold to separate CDX2^{neg} from CDX2^{pos} tumors may be the CDX2 expression value below which the frequency of objective clinical responses (OCR) following treatment with anti-EGFR monoclonal antibodies (e.g., cetuximab, panitumumab) is 0%. In another specific embodiment, the CDX2 expression level chosen as the threshold to separate CDX2^{neg} from CDX2^{pos} tumors may be the CDX2 expression value below which the progression-free survival (PFS) of patients treated with anti-EGFR monoclonal antibodies (e.g., cetuximab, panitumumab) is statistically undistinguishable from that of appropriate control patients that did not receive such treatment.

In one embodiment, in order to determine a subject's CDX2 expression level using mRNA expression data, e.g., from gene-expression microarray assays or other methods known in the art for mRNA detection, the values to stratify CDX2^{pos} and CDX2^{neg} tumors can be calculated using, for example, the StepMiner algorithm.

The StepMiner algorithm can be used to separate CDX2^{neg} from CDX2^{pos} samples, as described in, for example, Dalerba et al., NEJM, 374:211-222, 2016, the contents of which are hereby incorporated herein by reference. The experiments described therein were performed on a large database of gene-expression experiments publicly available from the National Center for Biotechnology Information (NCI) Gene-Expression Omnibus (GEO). This database contained gene-expression data collected using various Affymetrix platforms, including: 1) HG U133A [GPL96]; 2) HG U133 Plus 2.0 [GPL570]; 3) HG U133A 2.0 [GPL571]; 4) HT HG U133A [GPL3921]. The experiment described in Example 1 herein was performed on a public dataset (GSE5851), which was previously published (Khambata-Ford et al., Journal of Clinical Oncology, 25:3230-3237, 2007), and contains gene-expression data collected using the Affymetrix HG U133A 2.0 [GPL571] microarray platform.

In one embodiment, this method can also be applied to “binary” stratification (CDX2 negative expression level vs. CDX2 positive expression level) of gene-expression data collected on microarray platforms (e.g., Affymetrix, Illumina, Agilent, nanoString Technologies) and also on gene-expression data collected using different technological approaches (e.g., RealTime-qPCR, RNA-seq, and other gene expression methods known in the art or described herein). Thus, in one embodiment, the methods described herein to stratify colon cancer patients into CDX2 negative expression level vs. CDX2 positive expression level subgroups based on gene-expression measurements can be performed on the patient samples, e.g., tumor tissue samples, using any available analytical technique for measuring mRNA expression (e.g., gene-expression microarrays, RealTime-qPCR, RNA-seq), as described in detail herein.

With respect to determining a subject's CDX2 expression level using CDX2 protein expression data, in one embodiment, a semi-quantitative scoring system or scale (e.g., 0, 0.5, 2, 3) can be used to evaluate the intensity of the signal obtained on tumor tissues stained by immunohistochemistry. For example, the following scoring can be utilized:

Score 0 (no staining);

Score 0.5 (weak/scattered staining in a minority of cancer cells);

Score 2 (moderate/strong staining in a majority of cancer cells);

Score 3 (strong staining in all cancer cells).

In one embodiment, the scale evaluates nuclear staining intensity, and can be based on a subjective assessment of the strength of the nuclear staining across the sample (which incorporates both the percentage of CDX2 positive cells and the average intensity of their nuclear signal). This approach (i.e., a semi-quantitative scale) is commonly used to score immunohistochemistry results, in both research and diagnostic settings. This semi-quantitative assessment of immunohistochemistry results by an experienced pathologist remains one of the most reliable approaches available in clinical practice (Cross et al., Journal of Clinical Pathology, 54:385-90, 2001; Kraus et al., Modern Pathology, 25:869-876, 2012), as well as the cornerstone of diagnostic assays used to guide treatment choices in cancer patients. For example, in the case of breast cancer patients, the visual and semi-quantitative assessment of immunohistochemistry results by an experienced pathologist is used to define: a) the estrogen receptor (ER) status of the tumor, which is used to decide whether to administer anti-estrogen hormone therapy (Kraus et al., Modern Pathology, 25:869-876, 2012); and b) the presence of HER2 amplification in cancer cells, which is used to decide whether to administer anti-HER2 monoclonal antibodies, such as trastuzumab or pertuzumab (Slamon et al., NEJM, 344:783-792, 2001; Lehr et al., American Journal of Clinical Pathology, 115:814-822, 2001; Gianni et al., Lancet Oncology, 13:25-32, 2012). Borrisholt et al., Appl Immunohistochem Mol Morphol., 21:64-72 (2013), the contents of which are incorporated by reference herein, also describes currently available techniques to assess CDX2 expression by immunohistochemistry.

In another embodiment, objective and quantitative approaches to evaluate CDX2 protein expression level in tumor tissues can be used, including, for example, the computer-assisted image-analysis of tissue sections stained by immunohistochemistry (Sullivan and Chung, Clinical Colorectal Cancer, 7:172-177, 2008; Lehr et al., American Journal of Clinical Pathology, 115:814-822, 2001; Tuominen et al., Breast Cancer Res., 12:R56, 2010); and a direct measurement of protein concentration by mass spectrometry (Nuciforo et al., Molecular Oncology, 10:138-147, 2016).

In various embodiments of the methods of the present disclosure, various technological approaches are available for determination of expression levels of the disclosed genes, including, without limitation, RT-PCR, Real Time-q PCR, RNA-seq, microarrays, and serial analysis of gene expression (SAGE), which will be discussed in detail below. In particular embodiments, the expression level of each gene may be determined in relation to various features of the expression products of the gene including exons, introns, protein epitopes and protein activity.

Expression levels of the CDX2 surrogate biomarkers of the invention, as set forth in Table 1 and Table 2, can be determined using the same methods as described above with respect to CDX2.

### EGFR Inhibitors

The disclosed methods are aimed at utilizing certain biomarkers, namely CDX2 and surrogates thereof, alone or in combination with additional biomarkers, to predict response to anti-EGFR therapies in colorectal cancer patients. In one embodiment of the disclosed methods, the EGFR inhibitor is an anti-EGFR antibody, e.g., a monoclonal antibody. In a specific embodiment, the anti-EGFR antibody is cetuximab (Erbitux™; Bristol-Myers Squib) or panitumumab (Vectibix™; Amgen) or necitumumab (Portrazza™; Eli Lilly and Co.). Other monoclonal antibodies in clinical development include, for example, zalutumumab, nimotuzumab and matuzumab. In another embodiment, the anti-EGFR antibody comprises a combination or mixture of different monoclonal antibodies (e.g., an oligoclonal antibody) directed against the same or different epitopes of the EGFR molecule (e.g., MM-151; Merrimack Pharmaceuticals Inc.; Arena et al., Science Translational Medicine, 8:324ra14, 2016).

In another embodiment, an EGFR inhibitor is a small molecule. Gefitinib, erlotinib, and lapatinib are examples of such small molecule kinase inhibitors. In a more specific embodiment, the kinase inhibitor is selected from:

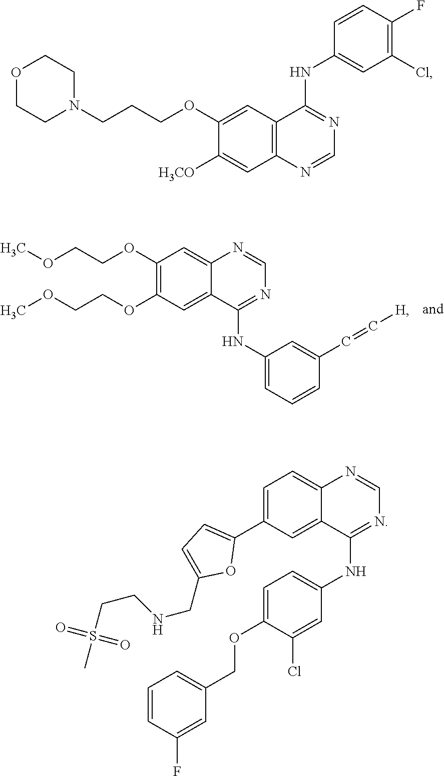

### Biomarker Detection

A biomarker used in the methods of the invention can be identified in a biological sample using any method known in the art. Determining the presence and/or level of one or more biomarker, e.g., protein or degradation product thereof, the presence and/or level of mRNA or pre-mRNA, or the presence and/or level of any biological molecule or product that is indicative of biomarker expression, or degradation product thereof, can be carried out for use in the methods of the invention by any method described herein or known in the art. In one embodiment, detection of the presence and/or level of one or more biomarker in the sample by a method described herein or known in the art transforms the sample.

### Protein Detection Techniques

Methods for the detection of expression and/or level of protein biomarkers are well known to those skilled in the art, and include but are not limited to, bead-based multiplexing technology, e.g., xMAP® technology (Luninex Corporation), microarrays, (e.g., protein microarrays), mass spectrometry techniques, mass cytometry techniques, such as CyTOF (Fluidigm) (see, Ornatsky, O., The Journal of Immunology, May 1, 2016, vol. 196 (1 Supplement)), 1-D or 2-D gel-based analysis systems, chromatography, enzyme linked immunosorbent assays (ELISAs), radioimmunoassays (RIA), enzyme immunoassays (EIA), western blotting, immunoprecipitation, and immunohistochemistry. In one embodiment, computer-assisted image-analysis of tissue sections stained by immunohistochemistry is used as described in, for example, Sullivan and Chung, Clinical Colorectal Cancer, 7:172-177, 2008; Lehr et al., American Journal of Clinical Pathology, 115:814-822, 2001; Tuominen et al., Breast Cancer Res., 12:R56, 2010). In another embodiment, a direct measurement of protein concentration by mass spectrometry is utilized (Nuciforo et al., Molecular Oncology, 10:138-147, 2016).

These methods use antibodies, or antibody equivalents, to detect protein. Antibody arrays, beads, or protein chips can also be employed, see for example U.S. Patent Application Nos. 20030013208A1; 20020155493A1, 20030017515 and U.S. Pat. Nos. 6,329,209 and 6,365,418, herein incorporated by reference in their entirety. ELISA and RIA procedures can be conducted such that a biomarker standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In one embodiment, a method for measuring biomarker expression includes the steps of: contacting a biological sample, e.g., a tissue, cell, or blood (e.g., serum) sample, with a reagent, e.g., an antibody or variant (e.g., fragment) thereof, which selectively binds the biomarker, thereby transforming the sample in a manner such that the level of expression of the biomarker is detected and quantified, e.g., by detecting whether the reagent is bound to the sample. A method can further include contacting the sample with a second reagent, e.g., antibody, e.g., a labeled antibody. The method can further include one or more steps of washing, e.g., to remove one or more reagents.

It can be desirable to immobilize one component of the assay system on a support, such as a bead, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art.

Enzymes employable for labeling are not particularly limited, but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

The xMAP technology (Luminex Corp.), and similar multiplexed bead-based systems can also be used to measure the expression of the biomarkers of the invention. This technology combines the principle of a sandwich immunoassay with fluorescent bead-based technology, allowing individual and multiplex analysis of many different analytes, e.g., up to 100, in a single microtiter well (see Vignali D A. Multiplexed particle-based flow cytometric assays. J Immunol Methods 2000; 243:243-55 and Yurkovetsky Z R, Kirkwood J M, Edington H D, et al. Clin Cancer Res. 2007; 13(8):2422-2428 for a detailed description).

Other techniques can be used to detect a biomarker according to a practitioner's preference based upon the present invention. One such technique is western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection can also be used.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that can be used for detecting and quantitating biomarker protein levels is western blotting. Cells can be frozen and homogenized in lysis buffer. Immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Antibodies against biomarkers can also be used for imaging purposes, for example, to detect the presence of a biomarker in a sample of a subject. Suitable labels include radioisotopes, iodine (¹²⁵I, ¹²¹I), carbon (¹⁴C), sulphur (³⁵S), tritium (³H), indium (¹¹²In), and technetium (^{99m}Tc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In some embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker). In one embodiment, the agent is a labeled or labelable antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample can contain heavy atoms (e.g., ¹³C) thereby permitting the test sample to be mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomarkers present in a sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomarker that is present in the first and second samples. A standard containing a known amount of a biomarker can be analyzed as the second sample to better quantify the amount of the biomarker present in the first sample. In certain embodiments, the identity of the biomarker in the first and second sample can also be determined.

### RNA Detection Techniques

Any method for qualitatively or quantitatively detecting a nucleic acid biomarker can be used. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). In one embodiment, the sample being tested is transformed when the nucleic acid biomarker is detected, e.g., by Northern blotting or by amplification of the biomarker in the sample, in a manner such that the level of expression of the biomarker is detected and quantified.

In one embodiment, quantitative real-time polymerase chain reaction (qRT-PCR) is used to evaluate mRNA levels of biomarker. In one specific embodiment, the levels of one or more biomarkers can be quantitated in a biological sample.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Another method for evaluation of biomarker expression is to detect mRNA levels of a biomarker by fluorescent in situ hybridization (FISH). FISH is a technique that can directly identify a specific region of DNA or RNA in a cell and therefore enables to visual determination of the biomarker expression in tissue samples. The FISH method has the advantages of a more objective scoring system and the presence of a built-in internal control consisting of the biomarker gene signals present in all non-neoplastic cells in the same sample. Fluorescence in situ hybridization is a direct in situ technique that is relatively rapid and sensitive. FISH test also can be automated.

Alternatively, mRNA expression can be detected on a DNA array, chip or a microarray.

Oligonucleotides corresponding to the biomarker(s) are immobilized on a chip which is then hybridized with labeled nucleic acids of a test sample obtained from a subject. Positive hybridization signal is obtained with the sample containing biomarker transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA can be extracted from the biological sample to be tested, reverse transcribed and fluorescent-labeled cDNA probes are generated. The microarrays capable of hybridizing to a biomarker, cDNA can then probed with the labeled cDNA probes, the slides scanned and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. Most preferably, the probe is directed to nucleotide regions unique to the particular biomarker RNA. The probes can be as short as is required to differentially recognize the particular biomarker mRNA transcripts, and can be as short as, for example, 15 bases; however, probes of at least 17 bases, more preferably 18 bases and still more preferably 20-50 bases are preferred. Preferably, the primers and probes hybridize specifically under stringent conditions to a nucleic acid fragment having the nucleotide sequence corresponding to the target gene. As herein used, the term “stringent conditions” means hybridization will occur only if there is at least 95% and preferably at least 97% identity between the sequences.

The form of labeling of the probes can be any that is appropriate, such as the use of radioisotopes, for example, ³²P and ³⁵S, or fluorescent probes, either alone or combined into specific sequences to create “optical barcodes” (Geiss et al., Nature Biotechnology, 26:317-325, 2008). Labeling with radioisotopes or fluorescent probes can be achieved, whether the probe is synthesized chemically or biologically, by the use of suitably labeled bases.

RNA levels can also be quantified using RNA-sequencing techniques (RNA-seq), which usually entail the preparation of a DNA library by reverse transcription of the RNA extracted from a certain biological sample, followed by the sequencing of individual cDNA molecules contained in the library, the counting of the number of times a certain sequence is found repeated in the library, and finally the calculation of the relative frequency of an individual sequence within the sample (e.g., the percentage of total sequences contained in the library represented by the sequence of interest). For an introduction to RNA-seq techniques, please refer to Wang et al., Nature Reviews in Genetics, 10:57-63, 2009.

### Reports

The methods of the present disclosure are suited for the preparation of reports summarizing the predictions resulting from the methods of the present disclosure. A “report,” as described herein, is an electronic or tangible document which includes report elements that provide information of interest relating to a likelihood assessment and its results. A subject report includes at least a likelihood assessment, e.g., an indication as to the likelihood that a cancer patient will exhibit a beneficial clinical response to an anti-EGFR treatment regimen. A subject report can be completely or partially electronically generated, e.g., presented on an electronic display (e.g., computer monitor). A report can further include one or more of: 1) information regarding the testing facility; 2) service provider information; 3) patient data; 4) sample data; 5) an interpretive report, which can include various information including: a) indication; b) test data, where test data can include a normalized level of one or more genes of interest, and 6) other features.

The present disclosure thus provides for methods of creating reports and the reports resulting therefrom. The report may include a summary of the expression levels of the RNA transcripts, or the expression products of such RNA transcripts, for certain genes in the cells obtained from the patient's tumor tissue sample or serum sample. The report may include a prediction that said subject has an increased likelihood of response to treatment with a particular therapy, e.g., anti-EGFR therapy, or the report may include a prediction that the subject has a decreased likelihood of response to the therapy, e.g., anti-EGFR therapy. The report may include a recommendation for treatment modality such as an EGFR inhibitor, surgery alone or surgery in combination with chemotherapy and/or radiation, or a combination thereof. The report may be presented in electronic format or on paper.

Thus, in some embodiments, the methods of the present disclosure further include generating a report that includes information regarding the patient's likelihood of response to therapy, particularly an anti-EGFR-based therapy. For example, the methods disclosed herein can further include a step of generating or outputting a report providing the results of a subject response likelihood assessment, which report can be provided in the form of an electronic medium (e.g., an electronic display on a computer monitor), or in the form of a tangible medium (e.g., a report printed on paper or other tangible medium).

A report that includes information regarding the likelihood that a patient will respond to treatment with therapy, particularly an anti-EGFR-based therapy, is provided to a user. An assessment as to the likelihood that a cancer patient will respond to treatment with a therapy, particularly an anti-EGFR-based therapy, is referred to below as a “response likelihood assessment” or, simply, “likelihood assessment.” A person or entity who prepares a report (“report generator”) can also perform the likelihood assessment. The report generator may also perform one or more of sample gathering, sample processing, and data generation, e.g., the report generator may also perform one or more of: a) sample gathering; b) sample processing; c) measuring a level of an indicator response gene product(s); d) measuring a level of a reference gene product(s); and e) determining a normalized level of a response indicator gene product(s). Alternatively, an entity other than the report generator can perform one or more sample gathering, sample processing, and data generation.

For clarity, it should be noted that the term “user,” which is used interchangeably with “client,” is meant to refer to a person or entity to whom a report is transmitted, and may be the same person or entity who does one or more of the following: a) collects a sample; b) processes a sample; c) provides a sample or a processed sample; and d) generates data (e.g., level of a biomarker; level of a reference gene product(s); normalized level of a biomarker for use in the likelihood assessment. In some cases, the person(s) or entity(ies) who provides sample collection and/or sample processing and/or data generation, and the person who receives the results and/or report may be different persons, but are both referred to as “users” or “clients” herein to avoid confusion. In certain embodiments, e.g., where the methods are completely executed on a single computer, the user or client provides for data input and review of data output. A “user” can be a health professional (e.g., a clinician, a laboratory technician, a physician (e.g., an oncologist, surgeon, pathologist), etc.).

In embodiments where the user only executes a portion of the method, the individual who, after computerized data processing according to the methods of the invention, reviews data output (e.g., results prior to release to provide a complete report, a complete, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

Where government regulations or other restrictions apply (e.g., requirements by health, malpractice, or liability insurance), all results, whether generated wholly or partially electronically, are subjected to a quality control routine prior to release to the user.

### Computer-Based Systems and Methods

The methods and systems described herein can be implemented in numerous ways. In one embodiment of particular interest, the methods involve use of a communications infrastructure, for example the internet. Several embodiments of the invention are discussed below. It is also to be understood that the present invention may be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site associated (e.g., at a service provider's facility).

For example, during or after data input by the user, portions of the data processing can be performed in the user-side computing environment. For example, the user-side computing environment can be programmed to provide for defined test codes to denote a likelihood “score,” where the score is transmitted as processed or partially processed responses to the reviewer's computing environment in the form of test code for subsequent execution of one or more algorithms to provide a result and/or generate a report in the reviewer's computing environment. The score can be a numerical score (representative of a numerical value) or a non-numerical score representative of a numerical value or range of numerical values (e.g., “A’ representative of a 90-95% likelihood of an outcome; “high” representative of a greater than 50% chance of response (or some other selected threshold of likelihood); “low” representative of a less than 50% chance of response (or some other selected threshold of likelihood); and the like.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which can include test data (e.g., level of a response indicator gene product(s); level of a reference gene product(s); normalized level of a response indicator gene product(s)); and may also include other data such as patient data. This information received can be stored at least temporarily in a database, and data analyzed to generate a report as described above.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In an embodiment of particular interest, all or a portion of the input data and/or all or a portion of the output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record which may exist in a confidential database at the healthcare facility.

A system for use in the methods described herein generally includes at least one computer processor (e.g., where the method is carried out in its entirety at a single site) or at least two networked computer processors (e.g., where data is to be input by a user (also referred to herein as a “client”) and transmitted to a remote site to a second computer processor for analysis, where the first and second computer processors are connected by a network, e.g., via an intranet or internet). The system can also include a user component(s) for input; and a reviewer component(s) for review of data, generated reports, and manual intervention. Additional components of the system can include a server component(s); and a database(s) for storing data (e.g., as in a database of report elements, e.g., interpretive report elements, or a relational database (RDB) which can include data input by the user and data output. The computer processors can be processors that are typically found in personal desktop computers (e.g., IBM, Dell, Macintosh), portable computers, mainframes, minicomputers, or other computing devices.

The networked client/server architecture can be selected as desired, and can be, for example, a classic two or three tier client server model. A relational database management system (RDMS), either as part of an application server component or as a separate component (RDB machine) provides the interface to the database.

In one example, the architecture is provided as a database-centric client/server architecture, in which the client application generally requests services from the application server which makes requests to the database (or the database server) to populate the report with the various report elements as required, particularly the interpretive report elements, especially the interpretation text and alerts. The server(s) (e.g., either as part of the application server machine or a separate RDB/relational database machine) responds to the client's requests.

The input client components can be complete, stand-alone personal computers offering a full range of power and features to run applications. The client component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe and offers data management, information sharing between clients, network administration and security. The application and any databases used can be on the same or different servers.

Other computing arrangements for the client and server(s), including processing on a single machine such as a mainframe, a collection of machines, or other suitable configuration are contemplated. In general, the client and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

### Kits

In non-limiting embodiments, the present invention provides for a kit for predicting whether a subject diagnosed with colorectal cancer is likely to be responsive or non-responsive to treatment with an EGFR inhibitor. The invention further provides for kits for determining the efficacy of a therapeutic agent, e.g., an anti-EGFR therapy, for treating colorectal cancer in a subject.

Types of kits include, but are not limited to, bead-based multiplexing technology, e.g., xMAP® technology (Luminex Corporation), packaged probe and primer sets (e.g. TaqMan probe/primer sets), arrays/microarrays, biomarker-specific antibodies and beads, which further contain one or more probes, primers or other detection reagents for detecting one or more biomarkers of the present invention.

In other non-limiting embodiments, a kit can comprise at least one antibody for immunodetection of the biomarker(s) to be identified, e.g., CDX2 alone or in combination with one or more surrogate biomarkers set forth in Table 1 or Table 2. Antibodies, both polyclonal and monoclonal, specific for a biomarker, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase).

In a further non-limiting embodiment, the biomarker-specific antibody can be provided bound to a solid support, such as a column matrix, an array, or well of a microtiter plate. Alternatively, the support can be provided as a separate element of the kit.

In a specific, non-limiting embodiment, a kit can comprise a pair of oligonucleotide primers suitable for polymerase chain reaction (PCR) or nucleic acid sequencing, for detecting one or more biomarker(s) to be identified. A pair of primers can comprise nucleotide sequences complementary to one or more biomarker of the invention. Alternatively, the complementary nucleotides can selectively hybridize to a specific region in close enough proximity 5′ and/or 3′ to the biomarker position to perform PCR and/or sequencing. Multiple biomarker-specific primers can be included in the kit to simultaneously assay large number of biomarkers. The kit can also comprise one or more polymerases, reverse transcriptase and nucleotide bases, wherein the nucleotide bases can be further detectably labeled.

In non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In a further non-limiting embodiment, the oligonucleotide primers can be immobilized on a solid surface or support, for example, on a nucleic acid microarray, wherein the position of each oligonucleotide primer bound to the solid surface or support is known and identifiable.

In certain non-limiting embodiments, a kit can comprise one or more reagents, e.g., primers, probes, microarrays, or antibodies, suitable for detecting expression levels of CDX2 and/or one or more surrogate biomarkers set forth in Table 1 or Table 2. In certain embodiments, the kit can further comprise one or more reagents, e.g., primers, probes, microarrays, or antibodies, suitable for detecting mutation status of any one or more additional biomarkers. Such additional biomarkers include, but are not limited to, KRAS, NRAS, BRAF, EGFR and PIK3CA.

A kit can further contain means for comparing the biomarker with a control or reference, and can include instructions for using the kit to detect the biomarker of interest. Specifically, the instructions describe that a lack of expression or low level of expression of CDX2 and/or one or more surrogate biomarkers set forth in Table 1 or Table 2, is indicative that the subject diagnosed with colorectal cancer is likely to be non-responsive to treatment with an EGFR inhibitor. Alternatively, a positive expression level of CDX2 and/or one or more surrogate biomarkers set forth in Table 1 or Table 2, is indicative that the subject diagnosed with colorectal cancer is likely to be responsive to treatment with an EGFR inhibitor.

Having described the invention, the same will be more readily understood through reference to the following Example, which is provided by way of illustration, and are not intended to limit the invention in any way. The contents of all references, GenBank Accession Numbers, patents and published patent applications cited throughout this application, as well as the Figures, are hereby incorporated by reference in their entirety.

### EXAMPLES

### Example 1: Boolean Logic Identifies CDX2 as a Predictive Biomarker for Responsiveness to Treatment of Human Colorectal Cancer with an EGFR Inhibitor

As shown herein, CDX2 was identified and its role was validated as a predictive biomarker, both at the gene and at the protein expression levels. Tumors lacking CDX2 expression responded to adjuvant chemotherapy, but not to the anti-EGFR monoclonal antibody cetuximab.

The methods disclosed below are also presented in Dalerba et al. “CDX2 as a Prognostic Biomarker in Stage II and Stage III Colon Cancer” N. Engl. J. Med. 2016, Vol 374, pp. 211-22, the contents of which are hereby incorporated by reference herein.

### Methods

### Bioinformatics Analysis of Gene-Expression Array Databases

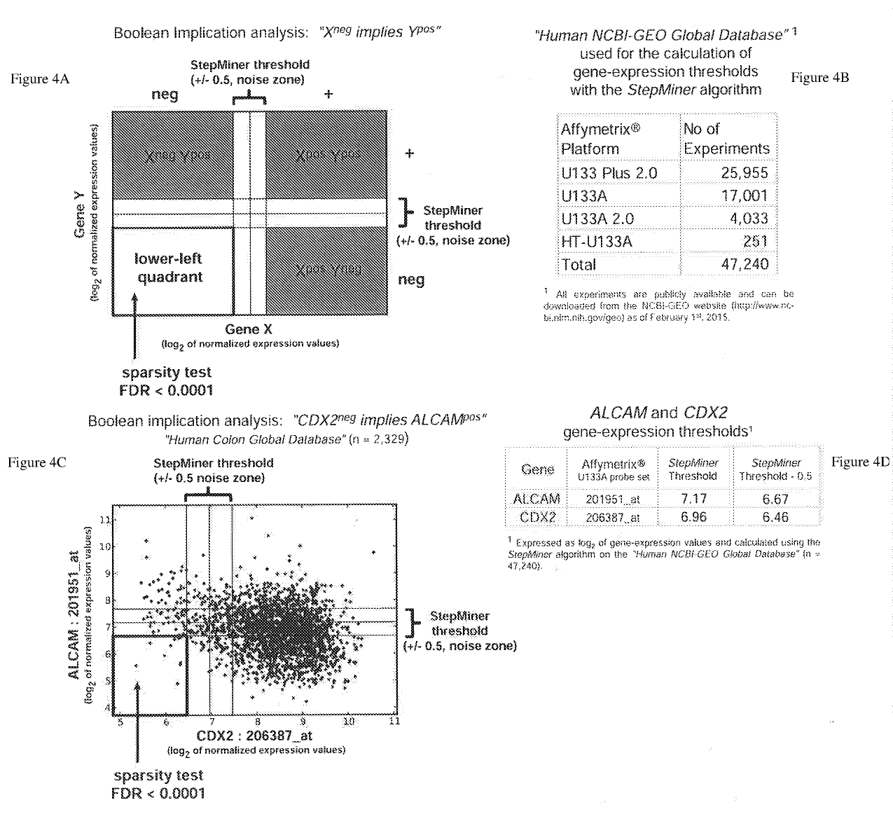

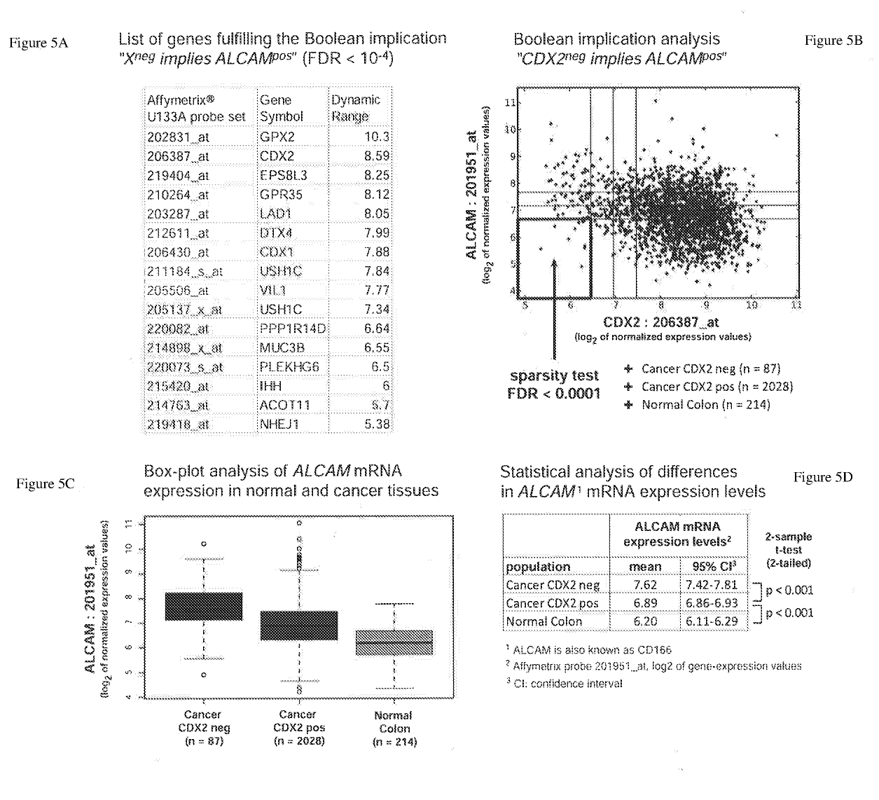

Genes that fulfilled the “X-negative implies ALCAM-positive” Boolean relationship were searched for in a collection of 2,329 human colon gene-expression array experiments. This collection was downloaded from the National Center for Biotechnology Information (NCBI) Gene Expression Omnibus (GEO) repository (www.ncbi.nlm.nih.gov/geo). The search was conducted with the use of BooleanNet software (Sahoo et al. Genome Biol. 2008; 9:R157) with a false discovery rate of less than 0.0001 as a cutoff point for positive results (FIG. 4). Candidate genes were ranked according to the dynamic range of their expression levels (FIG. 5).

The relationship between CDX2 expression levels and other molecular features such as microsatellite instability and TP53 mutations was studied in ad hoc collections annotated with the respective information after tumor samples were stratified into CDX2-negative and CDX2-positive subgroups with the use of the StepMiner algorithm²⁵. The relationship between CDX2 messenger RNA (mRNA) expression levels or ALCAM mRNA expression levels and disease-free survival was tested in a discovery data set of 466 patients. This data set was obtained by pooling four NCBI-GEO data sets (GSE14333, GSE17538, GSE31595, and GSE37892) (Jorissen R N, Clin Cancer Res 2009; 15:7642-7651; Smith J J, et al. Gastroenterology 2010; 138:958-968; Thorsteinsson M, K. et al. Int J Colorectal Dis 2012; 27:1579-1586; Laibe S, OMICS 2012; 16:560-565). Patients were stratified into negative-to-low (negative) and high (positive) subgroups with regard to CDX2 and ALCAM gene-expression levels with the use of the StepMiner algorithm, implemented within the Hegemon (Dalerba P, Kalisky T, Sahoo D, et al. Single-cell dissection of transcriptional heterogeneity in human colon tumors. Nat Biotechnol 2011; 29:1120-1127) software.

Experiments aimed at the evaluation of CDX2 as a predictive biomarker for response to the anti-EGFR monoclonal antibody cetuximab (FIGS. 1A-F) were performed on a gene-expression database which contains progression-free survival (PFS) information on 80 patients affected by metastatic colorectal carcinoma (AJCC Stage IV/Duke's Stage D) and homogeneously treated with cetuximab monotherapy (GSE5851; referred to as “Khambata-Ford database”) (Khambata-Ford S, et al. J Clin Oncol, 25:3230-7, 2007). All samples contained in this dataset were analyzed using the Affymetrix U133 A2.0 platform and were annotated with information related to the KRAS mutation status. In this case, given the fact that this dataset was the only one collected using the Affymetrix U133A 2.0 platform, to avoid bias due to application of StepMiner thresholds calculated on different, more commonly used, platforms (i.e., Affymetrix U133 Plus 2.0) the StepMiner thresholds were calculated using the GSE5851 dataset itself. As a result, all tumors whose CDX2 mRNA expression values were <1^{st} StepMiner threshold—0.5 were defined as CDX2^{neg} (CDX2: Affymetrix probe 206387_at <7.1).

An in-depth description of all bioinformatics procedures used in this study as well as complete lists of all NCBI-GEO sample number identifiers of individual gene-expression array experiments that were used to perform the various tests are provided in the Supplementary materials of Dalerba et al. N. Engl. J. Med. 2016, Vol 374, pp. 211-22.

### Immunohistochemical Testing

Formalin-fixed, paraffin-embedded tissue sections were stained with 4 mg per milliliter of a mouse antihuman CDX2 monoclonal antibody that was previously validated for diagnostic applications (clone CDX2-88, BioGenex). Li M K, Folpe A L. Adv Anat Pathol 2004; 11:101-105; Werling R W, et al. Am J Surg Pathol 2003; 27:303-310). The staining protocol was based on recommendations from the Nordic Immunohistochemical Quality Control organization (www.nordiqc.org), which suggests heat-induced antigen retrieval with Tris buffer and EDTA (pH 9.0) (Epitope Retrieval Solution pH9, Leica) (Borrisholt M, et al. Appl Immunohistochem Mol Morphol 2013; 21:64-72). Tissue slides were stained on a Bond-Max automatic stainer (Leica), and antigen detection was visualized with the use of the Bond Polymer Refine Detection kit (Leica).

### Analysis of CDX2 Protein Expression Levels in Tissue Microarrays (TMAs).

Colon-cancer tissue microarrays, fully annotated with clinical and pathological information, were obtained from three independent sources: 367 patients in the Cancer Diagnosis Program of the National Cancer Institute (NCI-CDP), 1519 patients in the National Surgical Adjuvant Breast and Bowel Project (NSABP) C-07 trial (NSABP C-07), and 321 patients in the Stanford Tissue Microarray Database (Stanford TMAD).

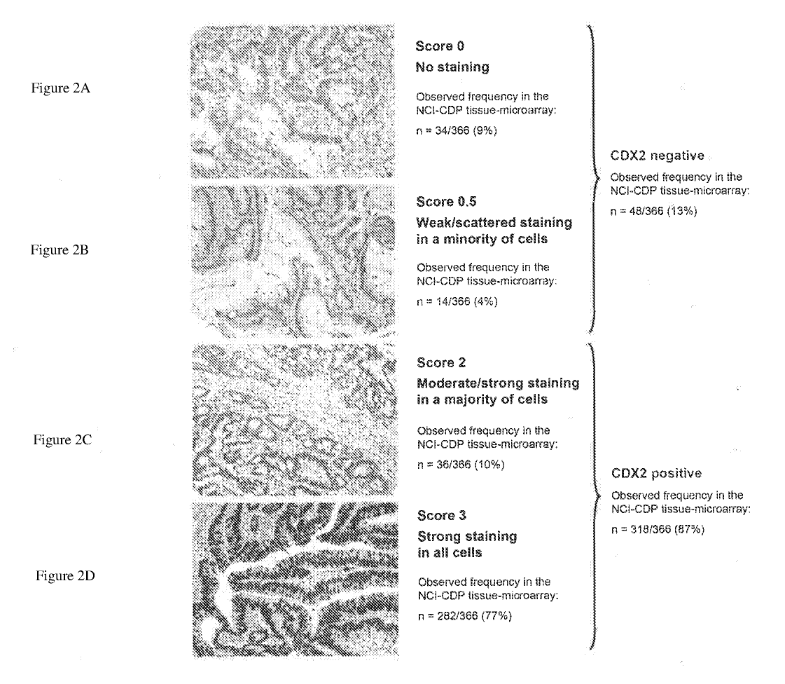

All tissue microarrays were scored for CDX2 expression in a blinded fashion. In cases in which tissue microarrays contained two tissue cores for a patient (i.e., two samples from distinct areas of the same tumor), the two cores were scored independently and paired at the end. If scores for the two samples were discordant, the final score for the tumor was upgraded to the higher score. A detailed description of the scoring system, together with representative photographs and scoring results, is provided in FIG. 2. All tumors in which the malignant epithelial component showed widespread nuclear expression of CDX2, either in all or a majority of cancer cells, were scored as CDX2^{pos}. All tumors in which the malignant epithelial component either completely lacked CDX2 expression or showed faint nuclear expression in a minority of malignant epithelial cells were scored as CDX2^{neg}.

The concordance between the scoring results obtained by two independent investigators was evaluated with the use of contingency tables and by calculation of Cohen's kappa indexes. The association between CDX2 expression and survival outcomes was tested by a third investigator who did not participate in the scoring process.

### Statistical Analysis

With respect to the role of CDX2 as a predictive biomarker for response to anti-EGFR monoclonal antibodies, once grouped based on gene or protein expression patterns, patient subsets were compared for survival outcomes, using both Kaplan-Meier survival curves and multivariate analysis based on the Cox proportional hazards method. Enrichment of high-grade carcinomas (G3/G4) in the CDX2^{neg} group was tested using Pearson's χ² test and by computing odds-ratios (OR) together with their 95% confidence intervals.

### Results

### Identification of CDX2

One aim of this study was to identify an actionable biomarker of poorly differentiated colon cancers (i.e., tumors depleted of mature colon epithelial cells). An actionable biomarker is one for which a clinical-grade diagnostic test had already been developed. Using a software algorithm designed for the discovery of genes with expression patterns that are linked by Boolean relationships (BooleanNet),²⁰ a database of 2329 human colon gene-expression array experiments was mined for genes that fulfilled the “X-negative implies ALCAM-positive” Boolean implication (i.e., genes with expression that was, at the same time, absent only in ALCAM-positive tumors and always present in ALCAM-negative tumors) (FIG. 4).

The search led to the identification of 16 candidate genes (FIG. 5). Of these genes, only 1 gene encoded a protein that could be studied by means of immunohistochemical analysis with the use of a clinical-grade diagnostic test: the homeobox transcription factor CDX2.^{28, 29, 31} CDX2 is a master regulator of intestinal development and oncogenesis,^{32, 33} and its expression is highly specific to the intestinal epithelium.²⁹ Colon cancers without CDX2 expression are often associated with an increased likelihood of aggressive features such as advanced stage, poor differentiation, vascular invasion, BRAF mutation, and the CpG island methylator phenotype (CIMP).³⁴⁻³⁹

A detailed analysis of the gene-expression relationship between CDX2 and ALCAM confirmed the existence of three gene-expression groups: CDX2-negative and ALCAM-positive, CDX2-positive and ALCAM-positive, and CDX2-positive and ALCAM-negative (FIG. 4). Lack of CDX2 expression was restricted to a small subgroup of 87 of 2115 colorectal cancers (4.1%). This subgroup was characterized by high levels of ALCAM expression (FIG. 5) and only partial overlap with tumors defined by microsatellite instability or TP53 mutations.

To evaluate whether CDX2^{neg} colon carcinomas can benefit from treatment with anti-EGFR monoclonal antibodies, the relationship between CDX2 mRNA expression and progression-free survival (PFS) was investigated in a cohort of Stage-IV colon cancer patients (n=80) who had been homogeneously treated with cetuximab monotherapy, and whose tumors' gene-expression data had been deposited in a public database (GSE5851; Khambata-Ford et al., J. Clin. Oncol., 25:3230-3237, 2007) archived within the National Center for Biotechnology Information's Gene Expression Omnibus (NCBI-GEO) repository. The cohort was stratified into CDX2^{neg} and CDX2^{pos} subgroups using the StepMiner algorithm (Sahoo et al., Nucleic Acids Res., 35:3705-3712, 2007), as described herein.

The results showed that CDX2^{neg} tumors were associated with reduced PFS as compared to CDX2^{pos} ones (FIGS. 1A and 1B). A more detailed analysis, performed after patient stratification according to both CDX2 expression and KRAS mutation status, revealed that the only patient subgroup who benefited from cetuximab treatment was CDX2^{pos}/KRAS^{wild-type} (FIGS. 1C and 1D). CDX2^{neg}/KRAs^{wild-type} tumors did not benefit from cetuximab treatment, despite their KRAS^{wild-type} status (FIGS. 1E and 1F).

Relationship Between CDX2 mRNA Expression, KRAS Mutation Status and Objective Tumor Response (OTR) and Disease Control (DC) Following Treatment with Cetuximab Across Two Colon Cancer Gene-Expression Datasets

To further confirm that CDX2 is predictive for responsiveness to treatment of colorectal cancer with an EGFR inhibitor, the frequency of (1) objective tumor response (OTR) and (2) disease control (DC) following treatment with cetuximab in CDX2^{pos} and CDX2^{neg} tumors in an expanded study population was investigated.

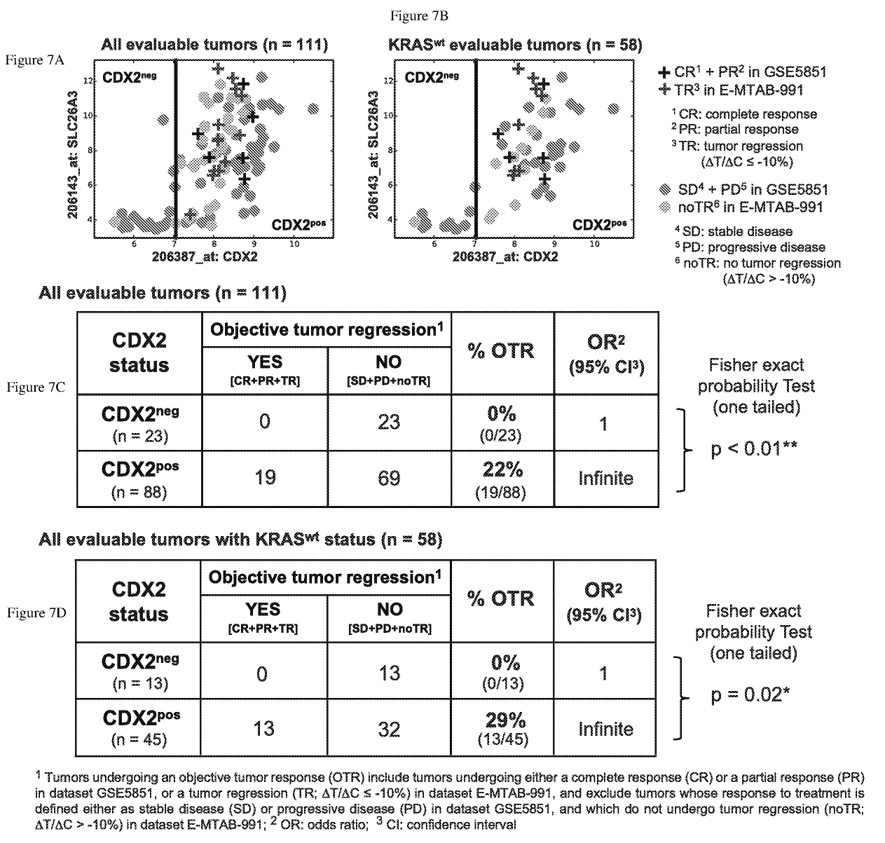

The relationship between CDX2 mRNA expression and objective tumor response (OTR) following treatment with anti-EGFR monoclonal antibodies was studied in a database of 111 independent colon carcinomas treated with cetuximab monotherapy (see FIGS. 7A-7D). The database was obtained by pooling two independent gene-expression array datasets: 1) GSE5851, downloaded from the NCBI-GEO public repository, and annotated with OTR information related to 68 primary tissue specimens from Stage-IV metastatic colon carcinomas (Khambata-Ford et al., J. Clin. Oncol., 25:3230-3237, 2007); and 2) E-MTAB-991, downloaded from the EMBL-ArrayExpress public repository, and annotated with OTR information related to 43 patient-derived xenograft (PDX) lines (Julien et al., Clin. Cancer Res., 18:5314-5328, 2012).

A visual exploration of the distribution of CDX2 and SLC26A3 mRNA expression levels across the two datasets, based on scatter-plots, revealed that tumors undergoing OTR were restricted to the CDX2^{pos} subgroup (FIGS. 7A and 7B). The association between CDX2 mRNA expression and OTR was tested for statistical significance using 2×2 contingency tables and Fisher's exact probability test, after stratification of tumors in CDX2^{neg} and CDX2^{pos} subgroups using the StepMiner algorithm (Dalerba et al., N. Engl. J. Med., 374:211-222, 2016). The results indicate that lack of CDX2 mRNA expression was associated with reduced OTR frequency, both across the whole database (FIG. 7C; p<0.01) and within the KRAS^{wt} subgroup (FIG. 7D; p=0.02), thus illustrating that CDX2 expression is predictive for responsiveness to treatment with an EGFR antibody.

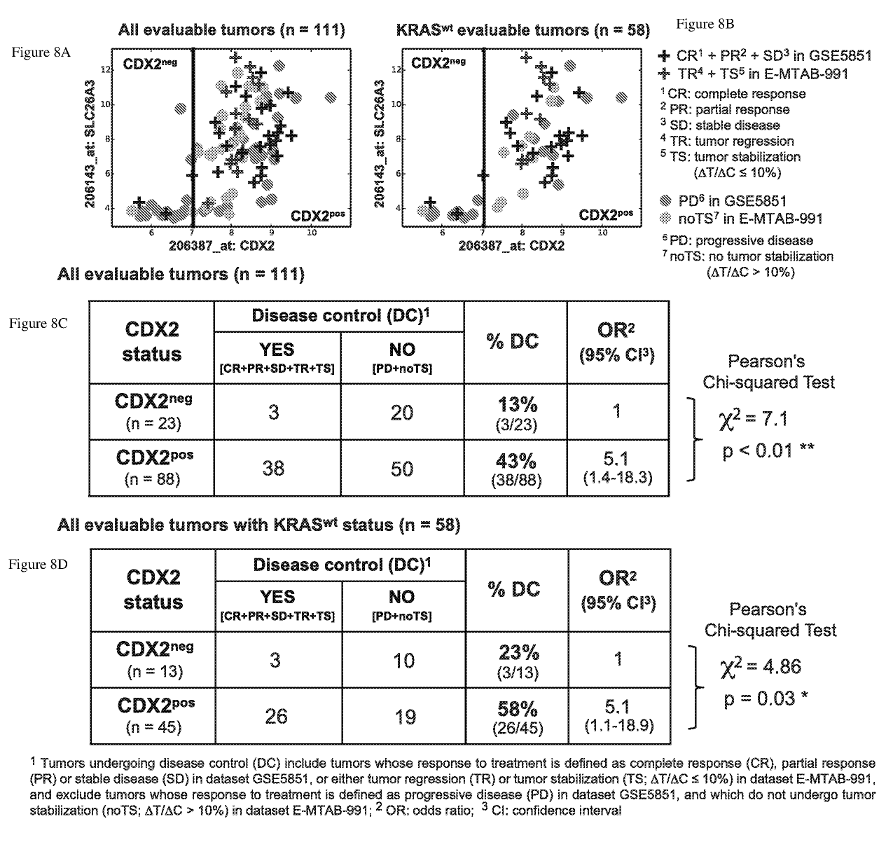

Likewise, the relationship between CDX2 mRNA expression and disease control (DC) following treatment with anti-EGFR monoclonal antibodies was studied in the above-described database of 111 independent colon carcinomas treated with cetuximab monotherapy (see FIGS. 8A-D). The database was obtained by pooling two independent gene-expression array datasets: 1) GSE5851, downloaded from the NCBI-GEO public repository, and annotated with DC information related to 68 primary tissue specimens from Stage-IV metastatic colon carcinomas (Khambata-Ford et al., J. Clin. Oncol., 25:3230-3237, 2007); and 2) E-MTAB-991, downloaded from the EMBL-ArrayExpress public repository, and annotated with DC information related to 43 patient-derived xenograft (PDX) lines (Julien et al., Clin. Cancer Res., 18:5314-5328, 2012).

A visual exploration of the distribution of CDX2 and SLC26A3 mRNA expression levels across the two datasets, based on scatter-plots, revealed that tumors undergoing DC were mostly found in the CDX2^{pos} subgroup (FIGS. 8A and 8B). The association between CDX2 mRNA expression and DC was tested for statistical significance using 2×2 contingency tables and the χ2 test, after stratification of tumors in CDX2^{neg} and CDX2^{pos} subgroups using the StepMiner algorithm (Dalerba et al., N. Engl. J. Med., 374:211-222, 2016). The results indicate that lack of CDX2 mRNA expression was associated with a reduced frequency of DC, both across the whole database (FIG. 8C; p<0.01) and within the KRAS′^{t} tumor subgroup (FIG. 8D; p=0.03), thus illustrating that CDX2 expression is predictive for responsiveness to treatment with an EGFR antibody.

### Example 2: Identification of Surrogate Biomarkers for CDX2

The inventors have identified genes that have expression patterns which are linearly correlated to CDX2, and, thus, can be used as surrogates of CDX2. Table 1, set forth below, includes a list of surrogate biomarkers whose mRNA expression levels were identified as positively correlated to those of CDX2 in a large database of human normal and cancerous colorectal tissues (n=1,832). Table 2, also set forth below, includes a list of surrogate biomarkers from Table 1 whose “high” expression levels associate with a statistically significant benefit from cetuximab monotherapy in KRAS^{wt} colon cancer patients.

The listing of markers set forth in Table 1 were generated using the database “Human Colon Global Database—GPL570,” described in Dalerba et al., NEJM, 374:211-222 (2016)—Supplementary Figure S3b. This database consists of 1,832 gene-expression array experiments generated using the Affymetrix® HG U133 Plus 2.0 [GPL570] microarray platform.

The listing of makers set forth in Table 2 was generated using the GSE5851 database (NCBI-GEO), described in Khambata-Ford et al., Journal of Clinical Oncology, 25:3230-3237 (2007).

For the generation of the listing of markers in Table 1, 0.40 was used as a threshold for a positive correlation coefficient (r), an approach adopted across various research fields (see, e.g., Ware and Gandek, Journal of Clinical Epidemiology, 51:945-952 (1998)). In Table 1, all correlation coefficients (r) are statistically significant for being different than r=0 (p<0.001), after Bonferroni correction for multiple comparisons.

For the generation of the listing of markers in Table 2, a statistical test was conducted for each of the genes listed in Table 1, after stratification of KRAS^{wt} tumors (n=43) included in the GSE5851 database into “high” and “low” expression groups, using the previously described StepMiner algorithm (Sahoo et al., Nucleic Acids Research, 35:3705-3712, 2007; Sahoo et al., Genome Biology, 9:R157, 2008; Dalerba et al., Nature Biotechnology, 29:1120-1127, 2011; Dalerba et al., NEJM, 374:211-222, 2016). Associations between “high” expression levels and improved progression-free survival (PFS) were tested for statistical significance using the log-rank test (p<0.05).

The database used to identify genes correlated to CDX2, as set forth in Table 1 (Human Colon Global Database—GPL570; Dalerba et al., NEJM, 374:211-222, 2016) is different from the database used to identify genes associated with response to cetuximab, as set forth in Table 2 (GSE5851; Khambata-Ford et al., Journal of Clinical Oncology, 25:3230-3237, 2007). In addition, the microarray platform used to generate the database utilized in Table 1 (GPL570) was designed to test for the expression of a larger number of genes than the microarray platform used to generate the database utilized in Table 2 (GPL571). Therefore, many genes (approximately 100) that are found as highly correlated to CDX2 in Table 1 cannot be tested for associations to benefit from treatment with cetuximab, because their expression levels are not available in the GSE5851 dataset. These genes, therefore, are not included in Table 2.

The surrogate biomarkers described in Table 1 and Table 2 can be used alone or in combination with CDX2 to assess and predict responsiveness of colorectal cancer to treatment with an EGFR inhibitor, e.g., cetuximab and panitumumab. For example, some of the genes included in Table 1 and Table 2 encode for proteins that can be “shed” by tumor cells in the bloodstream, and therefore can become measurable in the circulation, thus serving as serum biomarkers. A representative example of this class of proteins is CEACAM5 (also known as CEA), set forth in Table 1, which is detectable in the circulation of patients with metastatic colon cancer, and whose increasing levels can be used as a biomarker of tumor relapse in the monitoring of colon cancer patients who achieved a complete response, and who are being closely monitored for recurrence.

Therefore, the markers listed in Table 1 and Table 2 are useful as serum biomarkers for the prediction of tumor response to an EGFR inhibitor, e.g., cetuximab and panitumumab.

TABLE 1
Markers whose mRNA expression levels are positively correlated
(r > 0.40) to those of CDX2 in human colorectal tissues.^{a}

| Affymetrix ® | | | Correlation | p-value |
|---|---|---|---|---|
| probe set | Gene Symbol | Gene Name | to CDX2^{b} | correlation^{c} |
| 206387_at | CDX2 | caudal type homeobox 2 | 1.00 | p < 0.001 |
| 226961_at | PRR15 | proline rich 15 | 0.66 | p < 0.001 |
| 231606_at | — | CDNA FLJ20198 fis, clone COLF1083 | 0.61 | p < 0.001 |
| 225667_s_at | FAM84A /// | family with sequence similarity 84, | 0.60 | p < 0.001 |
| | LOC653602 | member A /// hypothetical | | |
| | | LOC653602 | | |
| 212338_at | MYO1D | myosin ID | 0.59 | p < 0.001 |
| 204039_at | CEBPA | CCAAT/enhancer binding protein | 0.59 | p < 0.001 |
| | | (C/EBP), alpha | | |
| 227736_at | C10orf99 | chromosome 10 open reading | 0.59 | p < 0.001 |
| | | frame 99 | | |
| 213435_at | SATB2 | SATB homeobox 2 | 0.58 | p < 0.001 |
| 229358_at | IHH | Indian hedgehog homolog | 0.58 | p < 0.001 |
| | | (Drosophila) | | |
| 227735_s_at | C10orf99 | chromosome 10 open reading | 0.58 | p < 0.001 |
| | | frame 99 | | |
| 220987_s_at | C11orf17 /// | chromosome 11 open reading | 0.58 | p < 0.001 |
| | NUAK2 | frame 17 /// NUAK family, SNF1- | | |
| | | like kinase, 2 | | |
| 205506_at | VIL1 | villin 1 | 0.58 | p < 0.001 |
| 218806_s_at | VAV3 | vav 3 guanine nucleotide exchange | 0.57 | p < 0.001 |
| | | factor | | |
| 220082_at | PPP1R14D | protein phosphatase 1, regulatory | 0.57 | p < 0.001 |
| | | (inhibitor) subunit 14D | | |
| 202525_at | PRSS8 | protease, serine, 8 | 0.57 | p < 0.001 |
| 209847_at | CDH17 | cadherin 17, LI cadherin (liver- | 0.56 | p < 0.001 |
| | | intestine) | | |
| 206430_at | CDX1 | caudal type homeobox 1 | 0.55 | p < 0.001 |
| 205311_at | DDC | dopa decarboxylase (aromatic L- | 0.55 | p < 0.001 |
| | | amino acid decarboxylase) | | |
| 210058_at | MAPK13 | mitogen-activated protein kinase 13 | 0.55 | p < 0.001 |
| 220073_s_at | PLEKHG6 | pleckstrin homology domain | 0.55 | p < 0.001 |
| | | containing, family G (with RhoGef | | |
| | | domain) members | | |
| 203953_s_at | CLDN3 | claudin 3 | 0.54 | p < 0.001 |
| 227867_at | LOC129293 | hypothetical protein LOC129293 | 0.54 | p < 0.001 |
| 214070_s_at | ATP10B | ATPase, class V, type 10B | 0.54 | p < 0.001 |
| 203559_s_at | ABP1 | amiloride binding protein 1 (amine | 0.53 | p < 0.001 |
| | | oxidase (copper-containing)) | | |
| 218322_s_at | ACSL5 | acyl-CoA synthetase long-chain | 0.53 | p < 0.001 |
| | | family member 5 | | |
| 218807_at | VAV3 | vav 3 guanine nucleotide exchange | 0.53 | p < 0.001 |
| | | factor | | |
| 209109_s_at | TSPAN6 | tetraspanin 6 | 0.53 | p < 0.001 |
| 215420_at | IHH | Indian hedgehog homolog | 0.53 | p < 0.001 |
| | | (Drosophila) | | |
| 206312_at | GUCY2C | guanylate cyclase 2C (heat stable | 0.52 | p < 0.001 |
| | | enterotoxin receptor) | | |
| 223423_at | GPR160 | G protein-coupled receptor 160 | 0.52 | p < 0.001 |
| 1568617_a_at | KIAA1543 | KIAA1543 | 0.52 | p < 0.001 |
| 222994_at | PRDX5 | peroxiredoxin 5 | 0.52 | p < 0.001 |
| 207202_s_at | NR1I2 | nuclear receptor subfamily 1, group | 0.52 | p < 0.001 |
| | | 1, member 2 | | |
| 209108_at | TSPAN6 | tetraspanin 6 | 0.52 | p < 0.001 |
| 1552281_at | SLC39A5 | solute carrier family 39 (metal ion | 0.52 | p < 0.001 |
| | | transporter), member 5 | | |
| 224221_s_at | VAV3 | vav 3 guanine nucleotide exchange | 0.52 | p < 0.001 |
| | | factor | | |
| 225129_at | CPNE2 | copine II | 0.52 | p < 0.001 |
| 223427_s_at | EPB41L4B | erythrocyte membrane protein | 0.52 | p < 0.001 |
| | | band 4.1 like 4B | | |
| 205892_s_at | FABP1 | fatty acid binding protein 1, liver | 0.51 | p < 0.001 |
| 220189_s_at | MGAT4B | mannosyl (alpha-1,3-)-glycoprotein | 0.51 | p < 0.001 |
| | | beta-1,4-N- | | |
| | | acetylglucosaminyltransferase, | | |
| | | isozyme B | | |
| 228912_at | VIL1 | villin 1 | 0.51 | p < 0.001 |
| 1487_at | ESRRA | estrogen-related receptor alpha | 0.51 | p < 0.001 |
| 212198_s_at | TM9SF4 | transmembrane 9 superfamily | 0.51 | p < 0.001 |
| | | protein member 4 | | |
| 210625_s_at | AKAP1 | A kinase (PRKA) anchor protein 1 | 0.51 | p < 0.001 |
| 235147_at | — | CDNA FLJ39330 fis, clone | 0.51 | p < 0.001 |
| | | OCBBF2016405 | | |
| 222592_s_at | ACSL5 | acyl-CoA synthetase long-chain | 0.51 | p < 0.001 |
| | | family member 5 | | |
| 209424_s_at | AMACR/// | alpha-methylacyl-CoA racemase/// | 0.51 | p < 0.001 |
| | C1QTNF3 | C1q and tumor necrosis factor | | |
| | | related protein 3 | | |
| 204433_s_at | SPATA2 | spermatogenesis associated 2 | 0.50 | p < 0.001 |
| 1553117_a_at | STK38 | serine/threonine kinase 38 | 0.50 | p < 0.001 |
| 220615_s_at | MLSTD1 | male sterility domain containing 1 | 0.50 | p < 0.001 |
| 234331_s_at | FAM84A | Family with sequence similarity 84, | 0.50 | p < 0.001 |
| | | member A | | |
| 202005_at | ST14 | suppression of tumorigenicity 14 | 0.50 | p < 0.001 |
| | | (colon carcinoma) | | |
| 209426_s_at | AMACR /// | alpha-methylacyl-CoA racemase /// | 0.50 | p < 0.001 |
| | C1QTNF3 | C1q and tumor necrosis factor | | |
| | | related protein 3 | | |
| 204130_at | HSD11B2 | hydroxysteroid (11-beta) | 0.50 | p < 0.001 |
| | | dehydrogenase 2 | | |
| 229396_at | OVOL1 | ovo-like 1(Drosophila) | 0.50 | p < 0.001 |
| 232977_x_at | MYH14 | myosin, heavy chain 14 | 0.49 | p < 0.001 |
| 220075_s_at | MUPCDH | mucin-like protocadherin | 0.49 | p < 0.001 |
| 213369_at | PCDH21 | protocadherin 21 | 0.49 | p < 0.001 |
| 207203_s_at | NR1I2 | nuclear receptor subfamily 1, group | 0.49 | p < 0.001 |
| | | 1, member 2 | | |
| 211184_s_at | USH1C | Usher syndrome 1C (autosomal | 0.49 | p < 0.001 |
| | | recessive, severe) | | |
| 234290_x_at | MYH14 | myosin, heavy chain 14 | 0.49 | p < 0.001 |
| 231941_s_at | MUC20 | mucin 20, cell surface associated | 0.49 | p < 0.001 |
| 211630_s_at | GSS | glutathione synthetase | 0.49 | p < 0.001 |
| 210859_x_at | CLN3 | ceroid-lipofuscinosis, neuronal 3, | 0.49 | p < 0.001 |
| | | juvenile (Batten, Spielmeyer-Vogt | | |
| | | disease) | | |
| 215702_s_at | CFTR | cystic fibrosis transmembrane | 0.49 | p < 0.001 |
| | | conductance regulator (ATP- | | |
| | | binding cassette sub-family C, | | |
| | | member 7) | | |
| 219946_x_at | MYH14 | myosin, heavy chain 14 | 0.49 | p < 0.001 |
| 220161_s_at | EPB41L4B | erythrocyte membrane protein | 0.49 | p < 0.001 |
| | | band 4.1 like 4B | | |
| 226988_s_at | MYH14 | myosin, heavy chain 14 | 0.48 | p < 0.001 |
| 244084_at | AIFM3 | apoptosis-inducing factor, | 0.48 | p < 0.001 |
| | | mitochondrion-associated, 3 | | |
| 225498_at | CHMP4B | chromatin modifying protein 4B | 0.48 | p < 0.001 |
| 1560587_s_at | PRDX5 | peroxiredoxin 5 | 0.48 | p < 0.001 |
| 202454_s_at | ERBB3 | v-erb-b2 erythroblastic leukemia | 0.48 | p < 0.001 |
| | | viral oncogene homolog 3 (avian) | | |
| 210264_at | GPR35 | G protein-coupled receptor 35 | 0.48 | p < 0.001 |
| 218094_s_at | DBNDD2 /// SYS1- | dysbindin (dystrobrevin binding | 0.48 | p < 0.001 |
| | DBNDD2 | protein 1) domain containing 2 /// | | |
| | | SYS1-DBNDD2 | | |
| 229889_at | C17orf76 | chromosome 17 open reading | 0.48 | p < 0.001 |
| | | frame 76 | | |
| 205137_x_at | USH1C | Usher syndrome 1C (autosomal | 0.48 | p < 0.001 |
| | | recessive, severe) | | |
| 228459_at | FAM84A | family with sequence similarity 84, | 0.48 | p < 0.001 |
| | | member A | | |
| 205929_at | GPA33 | glycoprotein A33 (transmembrane) | 0.48 | p < 0.001 |
| 205043_at | CFTR | cystic fibrosis transmembrane | 0.48 | p < 0.001 |
| | | conductance regulator (ATP- | | |
| | | binding cassette sub-family C, | | |
| | | member 7) | | |
| 232707_at | ISX | intestine-specific homeobox | 0.48 | p < 0.001 |
| 234312_s_at | ACSS2 | acyl-CoA synthetase short-chain | 0.48 | p < 0.001 |
| | | family member 2 | | |
| 225165_at | PPP1R1B | protein phosphatase 1, regulatory | 0.48 | p < 0.001 |
| | | (inhibitor) subunit 1B (dopamine | | |
| | | and cAMP regulated | | |
| | | phosphoprotein, DARPP-32) | | |
| 226622_at | MUC20 | mucin 20, cell surface associated | 0.48 | p < 0.001 |
| 220951_s_at | A1CF | APOBEC1 complementation factor | 0.48 | p < 0.001 |
| 209275_s_at | CLN3 | ceroid-lipofuscinosis, neuronal 3, | 0.48 | p < 0.001 |
| | | juvenile (Batten, Spielmeyer-Vogt | | |
| | | disease) | | |
| 209772_s_at | CD24 | CD24 molecule | 0.47 | p < 0.001 |
| 227642_at | TFCP2L1 | Transcription factor CP2-like 1 | 0.47 | p < 0.001 |
| 207180_s_at | HTATIP2 | HIV-1 Tat interactive protein 2, | 0.47 | p < 0.001 |
| | | 30 kDa | | |
| 223385_at | CYP2S1 | cytochrome P450, family 2, | 0.47 | p < 0.001 |
| | | subfamily S, polypeptide 1 | | |
| 226907_at | PPP1R14C | protein phosphatase 1, regulatory | 0.47 | p < 0.001 |
| | | (inhibitor) subunit 14C | | |
| 202925_s_at | PLAGL2 | pleiomorphic adenoma gene-like 2 | 0.47 | p < 0.001 |
| 219404_at | EPS8L3 | EPS8-like 3 | 0.47 | p < 0.001 |
| 227962_at | ACOX1 | acyl-Coenzyme A oxidase 1, | 0.47 | p < 0.001 |
| | | palmitoyl | | |
| 209790_s_at | CASP6 | caspase 6, apoptosis-related | 0.47 | p < 0.001 |
| | | cysteine peptidase | | |
| 221256_s_at | HDHD3 | haloacid dehalogenase-like | 0.47 | p < 0.001 |
| | | hydrolase domain containing 3 | | |
| 1561421_a_at | — | CDNA FLJ39484 fis, clone | 0.47 | p < 0.001 |
| | | PROST2014925 /// CDNA FLJ32697 | | |
| | | fis, clone TESTI2000372 | | |
| 225224_at | C20orf112 | chromosome 20 open reading | 0.47 | p < 0.001 |
| | | frame 112 | | |
| 213198_at | ACVR1B | activin A receptor, type IB | 0.47 | p < 0.001 |
| 214433_s_at | SELENBP1 | selenium binding protein 1 | 0.47 | p < 0.001 |
| 209144_s_at | CBFA2T2 | core-binding factor, runt domain, | 0.47 | p < 0.001 |
| | | alpha subunit 2; translocated to, 2 | | |
| 225000_at | PRKAR2A | Protein kinase, cAMP-dependent, | 0.47 | p < 0.001 |
| | | regulatory, type II, alpha | | |
| 216905_s_at | ST14 | suppression of tumorigenicity 14 | 0.46 | p < 0.001 |
| | | (colon carcinoma) | | |
| 218756_s_at | MGC4172 | short-chain | 0.46 | p < 0.001 |
| | | dehydrogenase/reductase | | |
| 203903_s_at | HEPH | hephaestin | 0.46 | p < 0.001 |
| 201674_s_at | AKAP1 | A kinase (PRKA) anchor protein 1 | 0.46 | p < 0.001 |
| 229427_at | SEMA5A | sema domain, seven | 0.46 | p < 0.001 |
| | | thrombospondin repeats (type 1 | | |
| | | and type 1-like), transmembrane | | |
| | | domain (TM) and short cytoplasmic | | |
| | | domain, (semaphorin) 5A | | |
| 1554006_a_at | LLGL2 | lethal giant larvae homolog 2 | 0.46 | p < 0.001 |
| | | (Drosophila) | | |
| 227348_at | PARS2 | prolyl-tRNA synthetase 2, | 0.46 | p < 0.001 |
| | | mitochondrial (putative) | | |
| 220376_at | LRRC19 | leucine rich repeat containing 19 | 0.46 | p < 0.001 |
| 209425_at | AMACR/// | alpha-methylacyl-CoA racemase /// | 0.46 | p < 0.001 |
| | C1QTNF3 | C1q and tumor necrosis factor | | |
| | | related protein 3 | | |
| 204272_at | LGALS4 | lectin, galactoside-binding, soluble, | 0.46 | p < 0.001 |
| | | 4 (galectin 4) | | |
| 232186_at | C20orf142 | chromosome 20 open reading | 0.46 | p < 0.001 |
| | | frame 142 | | |
| 211089_s_at | NEK3 | NIMA (never in mitosis gene a)- | 0.45 | p < 0.001 |
| | | related kinase 3 | | |
| 1555935_s_at | HUNK | hormonally upregulated Neu- | 0.45 | p < 0.001 |
| | | associated kinase | | |
| 230914_at | HNF4A | hepatocyte nuclear factor 4, alpha | 0.45 | p < 0.001 |
| 200861_at | CNOT1 | CCR4-NOT transcription complex, | 0.45 | p < 0.001 |
| | | subunit 1 | | |
| 230727_at | CISD3 | CDGSH iron sulfur domain 3 | 0.45 | p < 0.001 |
| 208651_x_at | CD24 | CD24 molecule | 0.45 | p < 0.001 |
| 229546_at | LOC653602 | hypothetical LOC653602 | 0.45 | p < 0.001 |
| 227055_at | METTL7B | methyltransferase like 7B | 0.45 | p < 0.001 |
| 219735_s_at | TFCP2L1 | transcription factor CP2-like 1 | 0.45 | p < 0.001 |
| 241547_at | — | CDNA FLJ26512 fis, clone | 0.45 | p < 0.001 |
| | | KDN07513 | | |
| 204798_at | MYB | v-myb myeloblastosis viral | 0.45 | p < 0.001 |
| | | oncogene homolog (avian) | | |
| 207747_s_at | DOK4 | docking protein 4 | 0.45 | p < 0.001 |
| 224799_at | NDFIP2 | Nedd4 family interacting protein 2 | 0.45 | p < 0.001 |
| 233979_s_at | ESPN | espin | 0.45 | p < 0.001 |
| 201884_at | CEACAM5 | carcinoembryonic antigen-related | 0.45 | p < 0.001 |
| | | cell adhesion molecule 5 | | |
| 224612_s_at | DNAJC5 | DnaJ (Hsp40) homolog, subfamily C, | 0.44 | p < 0.001 |
| | | member 5 | | |
| 216010_x_at | FUT3 | fucosyltransferase 3 (galactoside | 0.44 | p < 0.001 |
| | | 3(4)-L-fucosyltransferase, Lewis | | |
| | | blood group) | | |
| 227455_at | C6orf136 | chromosome 6 open reading frame | 0.44 | p < 0.001 |
| | | 136 | | |
| 226213_at | ERBB3 | v-erb-b2 erythroblastic leukemia | 0.44 | p < 0.001 |
| | | viral oncogene homolog 3 (avian) | | |
| 1555486_a_at | FLJ14213 | protor-2 | 0.44 | p < 0.001 |
| 218026_at | CCDC56 | coiled-coil domain containing 56 | 0.44 | p < 0.001 |
| 201675_at | AKAP1 | A kinase (PRKA) anchor protein 1 | 0.44 | p < 0.001 |
| 213953_at | KRT20 | keratin 20 | 0.44 | p < 0.001 |
| 205597_at | SLC44A4 | solute carrier family 44, member 4 | 0.44 | p < 0.001 |
| 212841_s_at | PPFIBP2 | PTPRF interacting protein, binding | 0.44 | p < 0.001 |
| | | protein 2 (liprin beta 2) | | |
| 214347_s_at | DDC | dopa decarboxylase (aromatic L- | 0.44 | p < 0.001 |
| | | amino acid decarboxylase) | | |
| 222470_s_at | UQCC | ubiquinol-cytochrome c reductase | 0.44 | p < 0.001 |
| | | complex chaperone, CBP3 homolog | | |
| | | (yeast) | | |
| 234850_at | MOGAT3 | monoacylglycerol O-acyltransferase 3 | 0.44 | p < 0.001 |
| 237338_at | B3GNT8 | UDP-GlcNAc: betaGal beta-1,3-N- | 0.44 | p < 0.001 |
| | | acetylglucosaminyltransferase 8 | | |
| 243669_s_at | PRAP1 | proline-rich acidic protein 1 | 0.44 | p < 0.001 |
| 225891_at | C9orf75 | chromosome 9 open reading frame | 0.44 | p < 0.001 |
| | | 75 | | |
| 202831_at | GPX2 | glutathione peroxidase 2 | 0.44 | p < 0.001 |
| | | (gastrointestinal) | | |
| 220041_at | PIGZ | phosphatidylinositol glycan anchor | 0.44 | p < 0.001 |
| | | biosynthesis, class Z | | |
| 223103_at | STARD10 | StAR-related lipid transfer (START) | 0.43 | p < 0.001 |
| | | domain containing 10 | | |
| 242414_at | QPRT | quinolinate | 0.43 | p < 0.001 |
| | | phosphoribosyltransferase | | |
| | | (nicotinate-nucleotide | | |
| | | pyrophosphorylase (carboxylating)) | | |
| 210390_s_at | CCL14 /// CCL15 | chemokine (C-C motif) ligand 14 /// | 0.43 | p < 0.001 |
| | | chemokine (C-C motif) ligand 15 | | |
| 232011_s_at | MAP1LC3A | microtubule-associated protein 1 | 0.43 | p < 0.001 |
| | | light chain 3 alpha | | |
| 212771_at | C10orf38 | chromosome 10 open reading | 0.43 | p < 0.001 |
| | | frame 38 | | |
| 207217_s_at | NOX1 | NADPH oxidase 1 | 0.43 | p < 0.001 |
| 203954_x_at | CLDN3 | claudin 3 | 0.43 | p < 0.001 |
| 211689_s_at | TMPRSS2 | transmembrane protease, serine 2 | 0.43 | p < 0.001 |
| 225860_at | LOC729580 | hypothetical LOC729580 | 0.43 | p < 0.001 |
| 58994_at | CC2D1A | coiled-coil and C2 domain | 0.43 | p < 0.001 |
| | | containing 1A | | |
| 223170_at | TMEM98 | transmembrane protein 98 | 0.43 | p < 0.001 |
| 221648_s_at | — | — | 0.43 | p < 0.001 |
| 201015_s_at | JUP | junction plakoglobin | 0.43 | p < 0.001 |
| 211885_x_at | FUT6 | fucosyltransferase 6 (alpha (1,3) | 0.43 | p < 0.001 |
| | | fucosyltransferase) | | |
| 244650_at | — | CDNA FLJ43660 fis, clone | 0.43 | p < 0.001 |
| | | SYNOV4004823 | | |
| 209690_s_at | DOK4 | docking protein 4 | 0.43 | p < 0.001 |
| 223961_s_at | CISH | cytokine inducible SH2-containing | 0.43 | p < 0.001 |
| | | protein | | |
| 212838_at | DNMBP | dynamin binding protein | 0.43 | p < 0.001 |
| 209679_s_at | LOC57228 | small trans-membrane and | 0.43 | p < 0.001 |
| | | glycosylated protein | | |
| 207259_at | C17orf73 | chromosome 17 open reading | 0.43 | p < 0.001 |
| | | frame 73 | | |
| 223438_s_at | PPARA | peroxisome proliferator-activated | 0.43 | p < 0.001 |
| | | receptor alpha | | |
| 209917_s_at | TP53AP1 | TP53 activated protein 1 | 0.43 | p < 0.001 |
| 206286_s_at | TDGF1 /// TDGF3 | teratocarcinoma-derived growth | 0.43 | p < 0.001 |
| | | factor 1 /// teratocarcinoma- | | |
| | | derived growth factor 3, | | |
| | | pseudogene | | |
| 211715_s_at | BDH1 | 3-hydroxybutyrate dehydrogenase, | 0.43 | p < 0.001 |
| | | type 1 | | |
| 1555175_a_at | PBLD | phenazine biosynthesis-like protein | 0.43 | p < 0.001 |
| | | domain containing | | |
| 212925_at | C19orf21 | chromosome 19 open reading | 0.43 | p < 0.001 |
| | | frame 21 | | |
| 203997_at | PTPN3 | protein tyrosine phosphatase, non- | 0.43 | p < 0.001 |
| | | receptor type 3 | | |
| 204800_s_at | DHRS12 | dehydrogenase/reductase (SDR | 0.43 | p < 0.001 |
| | | family) member 12 | | |
| 216032_s_at | ERGIC3 | ERGIC and golgi 3 | 0.43 | p < 0.001 |
| 218704_at | RNF43 | ring finger protein 43 | 0.43 | p < 0.001 |
| 233571_x_at | C20orf149 | chromosome 20 open reading | 0.43 | p < 0.001 |
| | | frame 149 | | |
| 225536_at | TMEM54 | transmembrane protein 54 | 0.43 | p < 0.001 |
| 224336_s_at | DUSP16 | dual specificity phosphatase 16 | 0.43 | p < 0.001 |
| 201271_s_at | RALY | RNA binding protein, autoantigenic | 0.42 | p < 0.001 |
| | | (hnRNP-associated with lethal | | |
| | | yellow homolog (mouse)) | | |
| 213490_s_at | MAP2K2 | mitogen-activated protein kinase | 0.42 | p < 0.001 |
| | | kinase 2 | | |
| 44790_s_at | C13orf18 /// | chromosome 13 open reading | 0.42 | p < 0.001 |
| | LOC728970 | frame 18 /// hypothetical | | |
| | | LOC728970 | | |
| 208650_s_at | CD24 | CD24 molecule | 0.42 | p < 0.001 |
| 202924_s_at | PLAGL2 | pleiomorphic adenoma gene-like 2 | 0.42 | p < 0.001 |
| 221610_s_at | STAP2 | signal transducing adaptor family | 0.42 | p < 0.001 |
| | | member 2 | | |
| 227706_at | SPIRE2 | spire homolog 2 (Drosophila) | 0.42 | p < 0.001 |
| 211916_s_at | MYO1A | myosin IA | 0.42 | p < 0.001 |
| 225440_at | AGPAT3 | 1-acylglycerol-3-phosphate O- | 0.42 | p < 0.001 |
| | | acyltransferase 3 | | |
| 230043_at | MUC20 | mucin 20, cell surface associated | 0.42 | p < 0.001 |
| 216379_x_at | CD24 | CD24 molecule | 0.42 | p < 0.001 |
| 209771_x_at | CD24 | CD24 molecule | 0.42 | p < 0.001 |
| 211882_x_at | FUT6 | fucosyltransferase 6 (alpha (1,3) | 0.42 | p < 0.001 |
| | | fucosyltransferase) | | |
| 219471_at | C13orf18 /// | chromosome 13 open reading | 0.42 | p < 0.001 |
| | LOC728970 | frame 18 /// hypothetical | | |
| | | LOC728970 | | |
| 239435_x_at | SHROOM1 | shroom family member 1 | 0.42 | p < 0.001 |
| 222257_s_at | ACE2 | angiotensin I converting enzyme | 0.42 | p < 0.001 |
| | | (peptidyl-dipeptidase A) 2 | | |
| 204044_at | QPRT | quinolinate | 0.42 | p < 0.001 |
| | | phosphoribosyltransferase | | |
| | | (nicotinate-nucleotide | | |
| | | pyrophosphorylase (carboxylating)) | | |
| 217289_s_at | SLC37A4 | solute carrier family 37 (glucose-6- | 0.42 | p < 0.001 |
| | | phosphate transporter), member 4 | | |
| 227994_x_at | C20orf149 | chromosome 20 open reading | 0.42 | p < 0.001 |
| | | frame 149 | | |
| 201131_s_at | CDH1 | cadherin 1, type 1, E-cadherin | 0.42 | p < 0.001 |
| | | (epithelial) | | |
| 202550_s_at | VAPB | VAMP (vesicle-associated | 0.42 | p < 0.001 |
| | | membrane protein)-associated | | |
| | | protein B and C | | |
| 219739_at | RNF186 | ring finger protein 186 | 0.42 | p < 0.001 |
| 210827_s_at | ELF3 | E74-like factor 3 (ets domain | 0.42 | p < 0.001 |
| | | transcription factor, epithelial- | | |
| | | specific) | | |
| 224613_s_at | DNAJC5 | DnaJ (Hsp40) homolog, subfamily C, | 0.42 | p < 0.001 |
| | | member 5 | | |
| 201425_at | ALDH2 | aldehyde dehydrogenase 2 family | 0.42 | p < 0.001 |
| | | (mitochondrial) | | |
| 224482_s_at | RAB11FIP4 | RAB11 family interacting protein 4 | 0.42 | p < 0.001 |
| | | (class II) | | |
| 218960_at | TMPRSS4 | transmembrane protease, serine 4 | 0.42 | p < 0.001 |
| 210398_x_at | FUT6 | fucosyltransferase 6 (alpha (1,3) | 0.42 | p < 0.001 |
| | | fucosyltransferase) | | |
| 201835_s_at | PRKAB1 | protein kinase, AMP-activated, beta | 0.42 | p < 0.001 |
| | | 1 non-catalytic subunit | | |
| 218010_x_at | C20orf149 | chromosome 20 open reading | 0.42 | p < 0.001 |
| | | frame 149 | | |
| 206000_at | MEP1A | meprin A, alpha (PABA peptide | 0.41 | p < 0.001 |
| | | hydrolase) | | |
| 1554260_a_at | FRYL | FRY-like | 0.41 | p < 0.001 |
| 204608_at | ASL | argininosuccinate lyase | 0.41 | p < 0.001 |
| 223425_at | RAVER1 | ribonucleoprotein, PTB-binding 1 | 0.41 | p < 0.001 |
| 209691_s_at | DOK4 | docking protein 4 | 0.41 | p < 0.001 |
| 211165_x_at | EPHB2 | EPH receptor B2 | 0.41 | p < 0.001 |
| 221572_s_at | SLC26A6 | solute carrier family 26, member 6 | 0.41 | p < 0.001 |
| 227743_at | LOC100134144 /// | myosin XVB pseudogene /// similar | 0.41 | p < 0.001 |
| | MYO15B | to KIAA1783 protein | | |
| 236279_at | — | Transcribed locus | 0.41 | p < 0.001 |
| 210010_s_at | SLC25A1 | solute carrier family 25 | 0.41 | p < 0.001 |
| | | (mitochondrial carrier; citrate | | |
| | | transporter), member 1 | | |
| 210117_at | SPAG1 | sperm associated antigen 1 | 0.41 | p < 0.001 |
| 232579_at | — | CDNA | 0.41 | p < 0.001 |
| 228123_s_at | ABHD12 | abhydrolase domain containing 12 | 0.41 | p < 0.001 |
| 212329_at | SCAP | SREBF chaperone | 0.41 | p < 0.001 |
| 207625_s_at | CBFA2T2 | core-binding factor, runt domain, | 0.41 | p < 0.001 |
| | | alpha subunit 2; translocated to, 2 | | |
| 212510_at | GPD1L | glycerol-3-phosphate | 0.41 | p < 0.001 |
| | | dehydrogenase 1-like | | |
| 203560_at | GGH | gamma-glutamyl hydrolase | 0.41 | p < 0.001 |
| | | (conjugase, | | |
| | | folylpolygammaglutamyl hydrolase) | | |
| 218910_at | TMEM16K | transmembrane protein 16K | 0.41 | p < 0.001 |
| 204231_s_at | FAAH | fatty acid amide hydrolase | 0.41 | p < 0.001 |
| 223094_s_at | ANKH | ankylosis, progressive homolog | 0.41 | p < 0.001 |
| | | (mouse) | | |
| 212181_s_at | NUDT4 /// | nudix (nucleoside diphosphate | 0.41 | p < 0.001 |
| | NUDT4P1 | linked moiety X)-type motif 4 /// | | |
| | | nudix (nucleoside diphosphate | | |
| | | linked moiety X)-type motif 4 | | |
| | | pseudogene 1 | | |
| 225510_at | OAF | OAF homolog (Drosophila) | 0.41 | p < 0.001 |
| 226553_at | PP9284 /// | transmembrane protease, serine 2/// | 0.41 | p < 0.001 |
| | TMPRSS2 | hypothetical protein | | |
| | | LOC100130534 | | |
| 238607_at | ZNF342 | zinc finger protein 342 | 0.41 | p < 0.001 |
| 223245_at | STRBP | spermatid perinuclear RNA binding | 0.41 | p < 0.001 |
| | | protein | | |
| 208937_s_at | ID1 | inhibitor of DNA binding 1, | 0.41 | p < 0.001 |
| | | dominant negative helix-loop-helix | | |
| | | protein | | |
| 200867_at | ZNF313 | zinc finger protein 313 | 0.41 | p < 0.001 |
| 219418_at | NHEJ1 | nonhomologous end-joining factor 1 | 0.41 | p < 0.001 |
| 205166_at | CAPN5 | calpain 5 | 0.41 | p < 0.001 |
| 225354_s_at | SH3BGRL2 | SH3 domain binding glutamic acid- | 0.41 | p < 0.001 |
| | | rich protein like 2 | | |
| 217736_s_at | EIF2AK1 | eukaryotic translation initiation | 0.41 | p < 0.001 |
| | | factor 2-alpha kinase 1 | | |
| 218290_at | PLEKHJ1 | pleckstrin homology domain | 0.41 | p < 0.001 |
| | | containing, family J member 1 | | |
| 209212_s_at | KLF5 | Kruppel-like factor 5 (intestinal) | 0.41 | p < 0.001 |
| 222451_s_at | ZDHHC9 | zinc finger, DHHC-type containing 9 | 0.41 | p < 0.001 |
| 200979_at | PDHA1 | pyruvate dehydrogenase | 0.41 | p < 0.001 |
| | | (lipoamide) alpha 1 | | |
| 204856_at | B3GNT3 | UDP-GlcNAc: betaGal beta-1,3-N- | 0.41 | p < 0.001 |
| | | acetylglucosaminyltransferase 3 | | |
| 206239_s_at | SPINK1 | serine peptidase inhibitor, Kazal | 0.41 | p < 0.001 |
| | | type 1 | | |
| 209588_at | EPHB2 | EPH receptor B2 | 0.41 | p < 0.001 |
| 212482_at | RMND5A | required for meiotic nuclear | 0.41 | p < 0.001 |
| | | division 5 homolog A (S. cerevisiae) | | |
| 1560010_a_at | FLJ32063 | hypothetical protein LOC150538 | 0.41 | p < 0.001 |
| 218657_at | RAPGEFL1 | Rap guanine nucleotide exchange | 0.41 | p < 0.001 |
| | | factor (GEF)-like 1 | | |
| 210808_s_at | NOX1 | NADPH oxidase 1 | 0.41 | p < 0.001 |
| 206141_at | MOCS3 | molybdenum cofactor synthesis 3 | 0.40 | p < 0.001 |
| 226494_at | KIAA1543 | KIAA1543 | 0.40 | p < 0.001 |
| 206754_s_at | CYP2B6 /// | cytochrome P450, family 2, | 0.40 | p < 0.001 |
| | CYP2B7P1 | subfamily B, polypeptide 6 /// | | |
| | | cytochrome P450, family 2, | | |
| | | subfamily B, polypeptide 7 | | |
| | | pseudogene 1 | | |
| 1555019_at | PCDH21 | protocadherin 21 | 0.40 | p < 0.001 |
| 204255_s_at | VDR | vitamin D (1,25-dihydroxyvitamin | 0.40 | p < 0.001 |
| | | D3) receptor | | |
| 213324_at | SRC | v-src sarcoma (Schmidt-Ruppin A-2) | 0.40 | p < 0.001 |
| | | viral oncogene homolog (avian) | | |
| 224832_at | DUSP16 | dual specificity phosphatase 16 | 0.40 | p < 0.001 |
| 225145_at | NCOA5 | nuclear receptor coactivator 5 | 0.40 | p < 0.001 |
| 206755_at | CYP2B6 | cytochrome P450, family 2, | 0.40 | p < 0.001 |
| | | subfamily B, polypeptide 6 | | |
| 205894_at | ARSE | arylsulfatase E (chondrodysplasia | 0.40 | p < 0.001 |
| | | punctata 1) | | |
| 221762_s_at | PCIF1 | PDX1 C-terminal inhibiting factor 1 | 0.40 | p < 0.001 |
| 226727_at | CISD3 | CDGSH iron sulfur domain 3 | 0.40 | p < 0.001 |
| 209261_s_at | NR2F6 | nuclear receptor subfamily 2, group | 0.40 | p < 0.001 |
| | | F, member 6 | | |
| 219041_s_at | REPIN1 | replication initiator 1 | 0.40 | p < 0.001 |
| 225177_at | RAB11FIP1 | RAB11 family interacting protein 1 | 0.40 | p < 0.001 |
| | | (class I) | | |
| 202951_at | STK38 | serine/threonine kinase 38 | 0.40 | p < 0.001 |
| 231667_at | SLC39A5 | solute carrier family 39 (metal ion | 0.40 | p < 0.001 |
| | | transporter), member 5 | | |
| 210651_s_at | EPHB2 | EPH receptor B2 | 0.40 | p < 0.001 |
| 205799_s_at | SLC3A1 | solute carrier family 3 (cystine, | 0.40 | p < 0.001 |
| | | dibasic and neutral amino acid | | |
| | | transporters, activator of cystine, | | |
| | | dibasic and neutral amino acid | | |
| | | transport), member 1 | | |
| 212194_s_at | TM9SF4 | transmembrane 9 superfamily | 0.40 | p < 0.001 |
| | | protein member 4 | | |
| 205698_s_at | MAP2K6 | mitogen-activated protein kinase | 0.40 | p < 0.001 |
| | | kinase 6 | | |
| 212548_s_at | FRYL | FRY-like | 0.40 | p < 0.001 |
| 64408_s_at | CALML4 | calmodulin-like 4 | 0.40 | p < 0.001 |
| 218261_at | AP1M2 | adaptor-related protein complex 1, | 0.40 | p < 0.001 |
| | | mu 2 subunit | | |
| 229777_at | CLRN3 | clarin 3 | 0.40 | p < 0.001 |

^{a}Correlation measured in the GPL570 sub-set of the Human Colon Global Database (n = 1,832) (Dalerba et al., NEJM, 374: 211-222 (2016) - Supplementary Table S3b);
^{b}Pearson correlation coefficient (r);
^{c}two-tailed t-test for correlation coefficients (null hypothesis: r = 0) after Bonferroni correction.

TABLE 2
List of genes whose mRNA expression levels are both positively correlated to those of CDX2
in human colorectal tissues (Table 1)^{a} and positively associated with improved progression-free survival
(PFS) following cetuximab monotherapy in KRAS^{wt} colorectal cancer patients.^{b}

| | | | | p-value |
|---|---|---|---|---|
| | | | | association |
| | | | | with |
| Affymetrix ® | | | Correlation | improved |
| probe set | Gene Symbol | Gene Name | to CDX2^{c} | PFS^{d} |
| 206387_at | CDX2 | caudal type homeobox 2 | 1.00 | 0.030 |
| 220987_s_at | C11orf17 /// | chromosome 11 open reading | 0.58 | 0.001 |
| | NUAK2 | frame 17 /// NUAK family, SNF1- | | |
| | | like kinase, 2 | | |
| 218806_s_at | VAV3 | vav 3 guanine nucleotide exchange | 0.57 | 0.003 |
| | | factor | | |
| 202525_at | PRSS8 | protease, serine, 8 | 0.57 | 0.012 |
| 210058_at | MAPK13 | mitogen-activated protein kinase | 0.55 | 0.030 |
| | | 13 | | |
| 203953_s_at | CLDN3 | claudin 3 | 0.54 | 0.027 |
| 214070_s_at | ATP10B | ATPase, class V, type 10B | 0.54 | 0.004 |
| 218807_at | VAV3 | vav 3 guanine nucleotide exchange | 0.53 | <0.001 |
| | | factor | | |
| 215420_at | IHH | Indian hedgehog homolog | 0.53 | 0.002 |
| | | (Drosophila) | | |
| 209108_at | TSPAN6 | tetraspanin 6 | 0.52 | 0.008 |
| 1487_at | ESRRA | estrogen-related receptor alpha | 0.51 | 0.049 |
| 212198_s_at | TM9SF4 | transmembrane 9 superfamily | 0.51 | 0.017 |
| | | protein member 4 | | |
| 210625_s_at | AKAP1 | A kinase (PRKA) anchor protein 1 | 0.51 | <0.001 |
| 204433_s_at | SPATA2 | spermatogenesis associated 2 | 0.50 | 0.002 |
| 202005_at | ST14 | suppression of tumorigenicity 14 | 0.50 | 0.004 |
| | | (colon carcinoma) | | |
| 211184_s_at | USH1C | Usher syndrome 1C (autosomal | 0.49 | 0.047 |
| | | recessive, severe) | | |
| 215702_s_at | CFTR | cystic fibrosis transmembrane | 0.49 | <0.001 |
| | | conductance regulator (ATP- | | |
| | | binding cassette sub-family C, | | |
| | | member 7) | | |
| 219946_x_at | MYH14 | myosin, heavy chain 14 | 0.49 | 0.024 |
| 218094_s_at | DBNDD2 /// SYS1- | dysbindin (dystrobrevin binding | 0.48 | 0.010 |
| | DBNDD2 | protein 1) domain containing 2 /// | | |
| | | SYS1-DBNDD2 | | |
| 205137_x_at | USH1C | Usher syndrome 1C (autosomal | 0.48 | 0.047 |
| | | recessive, severe) | | |
| 205043_at | CFTR | cystic fibrosis transmembrane | 0.48 | <0.001 |
| | | conductance regulator (ATP- | | |
| | | binding cassette sub-family C, | | |
| | | member 7) | | |
| 209275_s_at | CLN3 | ceroid-lipofuscinosis, neuronal 3, | 0.48 | 0.010 |
| | | juvenile (Batten, Spielmeyer-Vogt | | |
| | | disease) | | |
| 209772_s_at | CD24 | CD24 molecule | 0.47 | 0.010 |
| 202925_s_at | PLAGL2 | pleiomorphic adenoma gene-like 2 | 0.47 | <0.001 |
| 219404_at | EPS8L3 | EPS8-like 3 | 0.47 | 0.030 |
| 209144_s_at | CBFA2T2 | core-binding factor, runt domain, | 0.47 | 0.004 |
| | | alpha subunit 2; translocated to, 2 | | |
| 216905_s_at | ST14 | suppression of tumorigenicity 14 | 0.46 | 0.004 |
| | | (colon carcinoma) | | |
| 208651_x_at | CD24 | CD24 molecule | 0.45 | 0.047 |
| 219735_s_at | TFCP2L1 | transcription factor CP2-like 1 | 0.45 | 0.015 |
| 204798_at | MYB | v-myb myeloblastosis viral | 0.45 | 0.003 |
| | | oncogene homolog (avian) | | |
| 201675_at | AKAP1 | A kinase (PRKA) anchor protein 1 | 0.44 | 0.019 |
| 205597_at | SLC44A4 | solute carrier family 44, member 4 | 0.44 | 0.035 |
| 220041_at | PIGZ | phosphatidylinositol glycan anchor | 0.44 | 0.002 |
| | | biosynthesis, class Z | | |
| 58994_at | CC2D1A | coiled-coil and C2 domain | 0.43 | 0.011 |
| | | containing 1A | | |
| 209690_s_at | DOK4 | docking protein 4 | 0.43 | 0.025 |
| 212838_at | DNMBP | dynamin binding protein | 0.43 | <0.001 |
| 209679_s_at | LOC57228 | small trans-membrane and | 0.43 | 0.016 |
| | | glycosylated protein | | |
| 206286_s_at | TDGF1 /// TDGF3 | teratocarcinoma-derived growth | 0.43 | 0.026 |
| | | factor 1 /// teratocarcinoma- | | |
| | | derived growth factor 3, | | |
| | | pseudogene | | |
| 216032_s_at | ERGIC3 | ERGIC and golgi 3 | 0.43 | 0.001 |
| 218704_at | RNF43 | ring finger protein 43 | 0.43 | <0.001 |
| 201271_s_at | RALY | RNA binding protein, | 0.42 | 0.003 |
| | | autoantigenic (hnRNP-associated | | |
| | | with lethal yellow homolog | | |
| | | (mouse)) | | |
| 44790_s_at | C13orf18 /// | chromosome 13 open reading | 0.42 | <0.001 |
| | LOC728970 | frame 18 /// hypothetical | | |
| | | LOC728970 | | |
| 208650_s_at | CD24 | CD24 molecule | 0.42 | 0.047 |
| 216379_x_at | CD24 | CD24 molecule | 0.42 | 0.047 |
| 209771_x_at | CD24 | CD24 molecule | 0.42 | 0.047 |
| 219471_at | C13orf18 /// | chromosome 13 open reading | 0.42 | <0.001 |
| | LOC728970 | frame 18 /// hypothetical | | |
| | | LOC728970 | | |
| 202550_s_at | VAPB | VAMP (vesicle-associated | 0.42 | 0.003 |
| | | membrane protein)-associated | | |
| | | protein B and C | | |
| 210827_s_at | ELF3 | E74-like factor 3 (ets domain | 0.42 | 0.016 |
| | | transcription factor, epithelial- | | |
| | | specific) | | |
| 218960_at | TMPRSS4 | transmembrane protease, serine 4 | 0.42 | 0.019 |
| 201835_s_at | PRKAB1 | protein kinase, AMP-activated, | 0.42 | 0.004 |
| | | beta 1 non-catalytic subunit | | |
| 218010_x_at | C20orf149 | chromosome 20 open reading | 0.42 | 0.005 |
| | | frame 149 | | |
| 211165_x_at | EPHB2 | EPH receptor B2 | 0.41 | <0.001 |
| 200867_at | ZNF313 | zinc finger protein 313 | 0.41 | 0.028 |
| 217736_s_at | EIF2AK1 | eukaryotic translation initiation | 0.41 | 0.017 |
| | | factor 2-alpha kinase 1 | | |
| 209212_s_at | KLF5 | Kruppel-like factor 5 (intestinal) | 0.41 | 0.047 |
| 204856_at | B3GNT3 | UDP-GlcNAc: betaGal beta-1,3-N- | 0.41 | 0.003 |
| | | acetylglucosaminyltransferase 3 | | |
| 209588_at | EPHB2 | EPH receptor B2 | 0.41 | <0.001 |
| 213324_at | SRC | v-src sarcoma (Schmidt-Ruppin A- | 0.40 | 0.004 |
| | | 2) viral oncogene homolog (avian) | | |
| 219041_s_at | REPIN1 | replication initiator 1 | 0.40 | 0.013 |
| 202951_at | STK38 | serine/threonine kinase 38 | 0.40 | 0.001 |
| 210651_s_at | EPHB2 | EPH receptor B2 | 0.40 | 0.001 |

^{a}The correlation to CDX2 was measured in the GPL570 sub-set of the Human Colon Global Database (n = 1,832): Dalerba et al., NEJM, 374: 211-222 (2016) - Supplementary Table S3b;
^{b}The association with improved PFS was tested in the KRASwt subgroup of the GSE5851 public dataset (n = 43): Khambata-Ford et al., Journal of Clinical Oncology, 25: 3230-3237 (2007);
^{c}Pearson correlation coefficient (r);
^{d}Patients stratified in “high” vs. “low” expression groups using the StepMiner algorithm: Sahoo et al., Nucleic Acids Research, 35: 3705-3712 (2007).

### REFERENCES

1) André T, Boni C, Mounedji-Boudiaf L, et al. Oxaliplatin, fluorouracil, and leucovorin as adjuvant treatment for colon cancer. N Engl J Med 2004; 350:2343-2351.
2) Meyerhardt J A, Mayer R J. Systemic therapy for colorectal cancer. N Engl J Med 2005; 352:476-487.
3) Saltz L B, Cox J V, Blanke C, et al. Irinotecan plus fluorouracil and leucovorin for metastatic colorectal cancer. N Engl J Med 2000; 343:905-914.
4) O'Connor E S, Greenblatt D Y, LoConte N K, et al. Adjuvant chemotherapy for stage II colon cancer with poor prognostic features. J Clin Oncol 2011; 29:3381-3388.
5) Bardia A, Loprinzi C, Grothey A, et al. Adjuvant chemotherapy for resected stage II and III colon cancer: comparison of two widely used prognostic calculators. Semin Oncol 2010; 37:39-46.
6) Compton C, Fenoglio-Preiser C M, Pettigrew N, Fielding L P. American Joint Committee on Cancer Prognostic Factors Consensus Conference: Colorectal Working Group. Cancer 2000; 88:1739-1757.
7) Gill S, Loprinzi C L, Sargent D J, et al. Pooled analysis of fluorouracil-based adjuvant therapy for stage II and III colon cancer: who benefits and by how much? J Clin Oncol 2004; 22:1797-1806.
8) Meropol N J. Ongoing challenge of stage II colon cancer. J Clin Oncol 2011; 29:3346-3348.
9) Tournigand C, de Gramont A. Chemotherapy: is adjuvant chemotherapy an option for stage II colon cancer? Nat Rev Clin Oncol 2011; 8:574-576.
10) Barrier A, Boelle P Y, Roser F, et al. Stage II colon cancer prognosis prediction by tumor gene expression profiling. J Clin Oncol 2006; 24:4685-4691.
11) Wang Y, Jatkoe T, Zhang Y, et al. Gene expression profiles and molecular markers to predict recurrence of Dukes' B colon cancer. J Clin Oncol 2004; 22:1564-1571.
12) Jorissen R N, Gibbs P, Christie M, et al. Metastasis-associated gene expression changes predict poor outcomes in patients with Dukes stage B and C colorectal cancer. Clin Cancer Res 2009; 15:7642-7651.
13) Smith J J, Deane N G, Wu F, et al. Experimentally derived metastasis gene expression profile predicts recurrence and death in patients with colon cancer. Gastroenterology 2010; 138:958-968.
14) Yothers G, O'Connell M J, Lee M, et al. Validation of the 12-gene colon cancer recurrence score in NSABP C-07 as a predictor of recurrence in patients with stage II and III colon cancer treated with fluorouracil and leucovorin (FU/LV) and FU/LV plus oxaliplatin. J Clin Oncol 2013; 31:4512-4519.
15) Fang S H, Efron J E, Berho M E, Wexner S D. Dilemma of stage II colon cancer and decision making for adjuvant chemotherapy. J Am Coll Surg 2014; 219:1056-1069.
16) Grone J, Lenze D, Jurinovic V, et al. Molecular profiles and clinical outcome of stage UICC II colon cancer patients. Int J Colorectal Dis 2011; 26:847-858.
17) National Comprehensive Cancer Network. Clinical practice guidelines in oncology—colon cancer, version 3. 2015 (http://www.nccn.org).
18) Liu R, Wang X, Chen G Y, et al. The prognostic role of a gene signature from tumorigenic breast-cancer cells. N Engl J Med 2007; 356:217-226.
19) Merlos-Suarez A, Barriga F M, Jung P, et al. The intestinal stem cell signature identifies colorectal cancer stem cells and predicts disease relapse. Cell Stem Cell 2011; 8:511-524.
20) Sahoo D, Dill D L, Gentles A J, Tibshirani R, Plevritis S K. Boolean implication networks derived from large scale, whole genome microarray datasets. Genome Biol 2008; 9:R157-R157.
21) Dalerba P, Kalisky T, Sahoo D, et al. Single-cell dissection of transcriptional heterogeneity in human colon tumors. Nat Biotechnol 2011; 29:1120-1127.
22) Levin T G, Powell A E, Davies P S, et al. Characterization of the intestinal cancer stem cell marker CD166 in the human and mouse gastrointestinal tract. Gastroenterology 2010; 139)2072.e5-2082.e5.
23) Weichert W, Knosel T, Bellach J, Dietel M, Kristiansen G. ALCAM/CD166 is overexpressed in colorectal carcinoma and correlates with shortened patient survival. J Clin Pathol 2004; 57:1160-1164.
24) Dalerba P, Dylla S J, Park I K, et al. Phenotypic characterization of human colorectal cancer stem cells. Proc Natl Acad Sci USA 2007; 104:10158-10163.
25) Sahoo D, Dill D L, Tibshirani R, Plevritis S K. Extracting binary signals from microarray time-course data. Nucleic Acids Res 2007; 35:3705-3712.
26) Thorsteinsson M, Kirkeby L T, Hansen R, et al. Gene expression profiles in stages II and III colon cancers: application of a 128-gene signature. Int J Colorectal Dis 2012; 27:1579-1586.
27) Laibe S, Lagarde A, Ferrari A, Monges G, Birnbaum D, Olschwang S. A seven-gene signature aggregates a subgroup of stage II colon cancers with stage III. OMICS 2012; 16:560-565.
28) Li M K, Folpe A L. CDX-2, a new marker for adenocarcinoma of gastrointestinal origin. Adv Anat Pathol 2004; 11:101-105.
29) Werling R W, Yaziji H, Bacchi C E, Gown A M. CDX2, a highly sensitive and specific marker of adenocarcinomas of intestinal origin: an immunohistochemical survey of 476 primary and metastatic carcinomas. Am J Surg Pathol 2003; 27:303-310.
30) Borrisholt M, Nielsen S, Vyberg M. Demonstration of CDX2 is highly antibody dependant. Appl Immunohistochem Mol Morphol 2013; 21:64-72.
31) Kaimaktchiev V, Terracciano L, Tornillo L, et al. The homeobox intestinal differentiation factor CDX2 is selectively expressed in gastrointestinal adenocarcinomas. Mod Pathol 2004; 17:1392-1399.
32) Beck F, Stringer E J. The role of Cdx genes in the gut and in axial development. Biochem Soc Trans 2010; 38:353-357.
33) Chawengsaksophak K, James R, Hammond V E, Kontgen F, Beck F. Homeosis and intestinal tumours in Cdx2 mutant mice. Nature 1997; 386:84-87.
34) Hinoi T, Tani M, Lucas P C, et al. Loss of CDX2 expression and microsatellite instability are prominent features of large cell minimally differentiated carcinomas of the colon. Am J Pathol 2001; 159:2239-2248.
35) Lugli A, Tzankov A, Zlobec I, Terracciano L M. Differential diagnostic and functional role of the multi-marker phenotype CDX2/CK20/CK7 in colorectal cancer stratified by mismatch repair status. Mod Pathol 2008; 21:1403-1412.
36) Baba Y, Nosho K, Shima K, et al. Relationship of CDX2 loss with molecular features and prognosis in colorectal cancer. Clin Cancer Res 2009; 15:4665-4673.
37) Zlobec I, Bihl M P, Schwarb H, Terracciano L, Lugli A. Clinicopathological and protein characterization of BRAF- and K-RAS-mutated colorectal cancer and implications for prognosis. Int J Cancer 2010; 127:367-380.
38) Bae J M, Lee T H, Cho N Y, Kim T Y, Kang G H. Loss of CDX2 expression is associated with poor prognosis in colorectal cancer patients. World J Gastroenterol 2015; 21:1457-1467.
39) De Sousa E Melo F, Wang X, Jansen M, et al. Poor-prognosis colon cancer is defined by a molecularly distinct subtype and develops from serrated precursor lesions. Nat Med 2013; 19:614-618.
40) Altman D G, McShane L M, Sauerbrei W, Taube S E. Reporting Recommendations for Tumor Marker Prognostic Studies (REMARK): explanation and elaboration. PLoS Med 2012; 9:e1001216-e1001216.

1. A method of predicting whether a subject diagnosed with colorectal or rectal cancer is likely to be responsive or non-responsive to treatment with an EGFR inhibitor, comprising determining the subject's CDX2 expression level, and/or the expression level of one or more surrogate biomarkers set forth in Table 1 or Table 2, in a biological sample obtained from the subject; wherein a CDX2 positive expression level, and/or a positive expression level of one or more of the surrogate biomarkers, indicates that the subject is likely to be responsive to treatment with an EGFR inhibitor; and a CDX2 negative expression level, and/or a negative expression level of one or more of the surrogate biomarkers, indicates that the subject is likely to be non-responsive to treatment with an EGFR inhibitor.

2. A method of assessing the efficacy of an EGFR inhibitor for treating colorectal cancer in a subject prior to administration of the therapeutic agent, comprising determining the subject's CDX2 expression level, and/or the expression level of one or more surrogate biomarkers set forth in Table 1 or Table 2, in a biological sample obtained from the subject; and predicting that the EGFR inhibitor will be efficacious for treating colorectal cancer when the subject's CDX2 expression level is CDX2 positive, and/or the expression level of one or more of the surrogate biomarker is positive; and that the EGFR inhibitor will be non-efficacious for treating colorectal cancer when the subject's CDX2 expression level is CDX2 negative, and/or the expression level of one or more of the surrogate biomarkers is negative.

3. A method for excluding a subject diagnosed with colorectal cancer from treatment with an EGFR inhibitor, comprising determining the subject's CDX2 expression level, and/or the expression level of one or more surrogate biomarker set forth in Table 1 or Table 2, in a biological sample obtained from the subject, and excluding a subject from treatment with an EGFR inhibitor if the subject has a CDX2 negative expression level and/or the expression level of one or more of the surrogate biomarker is negative.

4. The method of claim 1, further comprising of treating colorectal cancer in a subject with an EGFR inhibitor when the subject's CDX2 expression level is CDX2 positive, and/or the expression level of one or more of the surrogate biomarkers is positive.

5.-8. (canceled)

9. The method of claim 1, further comprising analyzing the mutation status of one or more biomarkers selected from the group consisting of KRAS, NRAS, BRAF, EGFR and PIK3CA.

10.-14. (canceled)

15. The method of claim 9, further comprising determining whether a patient with or without one or more mutations in the KRAS, NRAS, BRAF, EGFR or PIK3CA genes would benefit from therapy with one or more of a BRAF inhibitor, a MEK inhibitor, an ERK inhibitor and an EGFR inhibitor, wherein the subject would benefit from the therapy when the subject's CDX2 expression level is CDX2 positive, or surrogate biomarker expression level is positive, and the subject would not benefit from the therapy when the subject's CDX2 expression level is CDX2 negative, or surrogate biomarker expression level is negative.

16. (canceled)

17. The method of claim 1, further comprising obtaining a biological sample from the subject.

18. The method of claim 17, wherein the biological sample is a colorectal tumor sample or a serum sample.

19.-20. (canceled)

21. The method of claim 1, wherein a positive CDX2 expression level or a positive surrogate biomarker expression level is indicated by a measureable level of CDX2 expression, or surrogate biomarker expression, in the biological sample.

22.-35. (canceled)

36. The method of claim 1, wherein a negative CDX2 expression level or negative surrogate biomarker expression level is indicated by a lack of CDX2 expression, or lack of surrogate biomarker expression, in the biological sample.

37. (canceled)

38. (canceled)

39. The method of claim 1, wherein the subject is a human.

40. The method of claim 1, wherein the subject's CDX2 expression level, or surrogate biomarker expression level, is determined by measuring the level of CDX2 or surrogate biomarker protein expression in the biological sample.

41.-43. (canceled)

44. The method of claim 40, wherein the level of CDX2 protein expression or surrogate biomarker protein expression is determined by immunohistochemistry, ELISA, HPLC/UV-Vis spectroscopy, mass spectrometry, mass cytometry, NMR, or any combination thereof.

45.-47. (canceled)

48. The method of claim 1, wherein the subject's CDX2 or surrogate biomarker expression level is determined by determining the level of its corresponding mRNA in the biological sample.

49.-51. (canceled)

52. The method of claim 1, wherein the EGFR inhibitor is an anti-EGFR antibody.

53. The method of claim 52, wherein the anti-EGFR antibody is cetuximab or panitumumab.

54. The method of claim 1, wherein the EGFR inhibitor is a small molecule.

55.-72. (canceled)

73. A kit for predicting whether a subject diagnosed with colorectal cancer is likely to be responsive or non-responsive to treatment with an EGFR inhibitor or assessing the efficacy of a therapeutic agent for treating colorectal cancer, comprising reagents useful for determining the subject's CDX2 expression level, and/or one or more surrogate biomarker expression level, in a biological sample from the subject.

74. (canceled)

75. The kit of claim 73, comprising at least one monoclonal antibody or antigen-binding fragment thereof, that specifically binds with CDX2, and/or one or more surrogate biomarkers, for determining the subject's CDX2 expression level and/or surrogate biomarker expression level.

76. The kit of claim 73, further comprising reagents useful for detecting one or more biomarkers selected from the group consisting of KRAS, NRAS, BRAF, EGFR and PIK3CA.

77.-86. (canceled)
